(19) United States
(12) Patent Application Publication
(10) Pub. No.: US 2010/0048638 A1
(43) Pub. Date: Feb. 25, 2010

(54) HEPARAN SULFATE INHIBITORS

(75) Inventors: Brett E. CRAWFORD, Poway, CA (US); Charles A. GLASS, San Diego, CA (US); Jillian R. BROWN, Poway, CA (US); Robert G. WITT, La Jolla, CA (US); Benedikt VOLLRATH, San Diego, CA (US); Jay LICHTER, Rancho Santa Fe, CA (US)

Correspondence Address: WILSON, SONSINI, GOODRICH & ROSATI, 650 PAGE MILL ROAD, PALO ALTO, CA, 94304-1050, US

(73) Assignee: ZACHARON PHARMACEUTICALS, INC., San Diego, CA (US)

(21) Appl. No.: 12/496,548

(22) Filed: Jul. 1, 2009

Related U.S. Application Data
(60) Provisional application No. 61/077,448, filed on Jul. 1, 2008.
(60) Provisional application No. 61/159,976, filed on Mar. 13, 2009.
(60) Provisional application No. 61/164,286, filed on Mar. 27, 2009.

(51) Int. Cl. A61K 31/4439; C07D 401/04; A61P 35/00

(52) U.S. Cl. 514/341; 546/272.7

(57) ABSTRACT

Provided herein are heparan sulfate inhibitors, including modulators of heparan sulfate glycosylation, heparan sulfate sulfation, and/or heparan sulfate epimerization.

### CROSS REFERENCE

This application claims the benefit of U.S. Provisional Application Nos. 61/077,448, filed Jul. 1, 2008, 61/159,976, filed Mar. 13, 2009, and 61/164,286, filed Mar. 27, 2009, which applications are incorporated herein by reference in their entirety.

### STATEMENT AS TO FEDERALLY SPONSORED RESEARCH

Certain inventions described herein were made with the support of the United States government under Contract 1 R43 CA112794 by the National Institutes of Health.

### BACKGROUND OF THE INVENTION

Heparan sulfate (HS) is a glycan found in mammals comprising glucosamine and uronic acid groups. In certain instances, heparan sulfate is bound to a core protein via a linkage tetrasaccharide, which generally has the structure -GlcAβ3Galβ3Galβ4Xylβ-O—.

### SUMMARY OF THE INVENTION

Provided in certain embodiments, herein is a process for modifying the structure of a glycosaminoglycan (e.g., heparan sulfate) on a core protein, comprising contacting a cell that translationally produces at least one core protein having at least one attached glycosaminoglycan (e.g., heparan sulfate) moiety with a selective inhibitor of glycosaminoglycan (e.g., heparan sulfate) biosynthesis, including a heparan sulfate glycosyltransferase, a heparan sulfate sulfotransferase, a heparan sulfate phosphotransferase, or a heparan sulfate epimerase.

In some embodiments, the selective inhibitor of a heparan sulfate sulfotransferase is an inhibitor of a heparan sulfate O-sulfotransferase, a heparan sulfate N-sulfotransferase, or a combination thereof. In specific embodiments, the inhibitor of a heparan sulfate O-sulfotransferase inhibits the 6-OH sulfation of a glucosylamine moiety, the 3-OH sulfation of a glucosylamine moiety, the 2-OH sulfation of a uronic acid moiety, the 6-O sulfation of a galactose moiety, or a combination thereof. In some embodiments, the inhibitor of a heparan sulfate glycosyltransferase inhibits the synthesis of the linkage region, the modification of the linkage region, the initiation of heparan sulfate synthesis, the synthesis of heparan sulfate, or a combination thereof. In some embodiments, the selective inhibitor of heparan sulfate is a selective inhibitor of glycosaminoglycans, the selectivity being relative to one or more non-glycosaminoglycan glycan, e.g., relative to extracellular glycans (e.g., N-linked glycans). In specific embodiments, a selective inhibitor of heparan sulfate selectively inhibits FGF binding (e.g., to glycosaminoglycans, including, heparan sulfate) in a cell, when the cell is contacted with the selective modulator of heparan sulfate, relative to lectin (e.g., Phaseolus Vulgaris (PHA)) binding of N-linked glycans in a cell.

Provided in some embodiments herein is a process of inhibiting heparan sulfate function in a cell comprising contacting the cell with a selective modulator of a heparan sulfate glycosyltransferase, a modulator of a heparan sulfate epimerase, or a modulator of a heparan sulfate sulfotransferase. In certain embodiments, the heparan sulfate function inhibited is an ability to bind a heparan sulfate binding lectin. In specific embodiments, the heparan sulfate lectin is a growth factor. In more specific embodiments, the growth factor is a fibroblast growth factor (FGF) or a vascular endothelia growth factor (VEGF). In some embodiments, the modulator of heparan sulfate sulfotransferase is an inhibitor of heparan sulfate sulfotransferase. In specific embodiments, the inhibitor of heparan sulfate sulfotransferase is an inhibitor of heparan sulfate O-sulfotransferase, heparan sulfate N-sulfotransferase, or a combination thereof. In more specific embodiments, the inhibitor of heparan sulfate O-sulfotransferase inhibits the 6-OH sulfation of a glucosylamine moiety, the 3-OH sulfation of a glucosylamine moiety, the 2-OH sulfation of a uronic acid moiety, or a combination thereof. In some embodiments, the modulator of a heparan sulfate sulfotransferase is a promoter of the heparan sulfate sulfotransferase. In certain embodiments, the modulator of a heparan sulfate epimerase is an inhibitor of the heparan sulfate epimerase. In some embodiments, the modulator of a heparan sulfate epimerase is a promoter of the heparan sulfate epimerase. In certain embodiments, the modulator of a heparan sulfate glycosyltransferase is an inhibitor of the heparan sulfate glycosyltransferase. In some embodiments, the modulator of a heparan sulfate glycosyltransferase is a promoter of the heparan sulfate glycosyltransferase. In certain embodiments, the cell is present in a human diagnosed with cancer.

Provided in some embodiments herein is a process of inhibiting heparan sulfate function in a cell comprising contacting the cell with a selective modulator of heparan sulfate biosynthesis. In certain embodiments, the selective modulator of heparan sulfate biosynthesis inhibits heparan glycosylation. In some embodiments, the selective modulator of heparan sulfate biosynthesis inhibits sulfation of heparan. In certain embodiments, the selective modulator of heparan sulfate biosynthesis promotes sulfation of heparan. In some embodiments, the selective modulator of heparan sulfate biosynthesis inhibits epimerization of heparan. In certain embodiments, the selective modulator of heparan sulfate biosynthesis promotes epimerization of heparan. In further or alternative embodiments, the selective modulator of heparan sulfate biosynthesis has a molecular weight of less than 1,000 g/mol.

Provided in certain embodiments herein is a method of treating cancer comprising administering a therapeutically effective amount of a selective modulator of heparan sulfate glycosylation, a modulator of heparan sulfate sulfation, or a selective modulator of heparan sulfate epimerization. In some embodiments, the selective modulator of heparan sulfate biosynthesis inhibits heparan glycosylation. In certain embodiments, the selective modulator of heparan sulfate biosynthesis inhibits sulfation of heparan. In some embodiments, the selective modulator of heparan sulfate biosynthesis promotes sulfation of heparan. In certain embodiments, the selective modulator of heparan sulfate biosynthesis inhibits epimerization of heparan. In some embodiments, the selective modulator of heparan sulfate biosynthesis promotes epimerization of heparan. In further or alternative embodiments, the selective modulator of heparan sulfate biosynthesis has a molecular weight of less than 1,000 g/mol.

Provided in certain embodiments herein is a method of treating a lysosomal storage disease comprising administering a therapeutically effective amount of a selective modulator of heparan sulfate glycosylation, a modulator of heparan sulfate sulfation, or a selective modulator of heparan sulfate epimerization. In some embodiments, the lysosomal storage disease is selected from mucopolysaccharidosis. In further or alternative embodiments, the selective modulator of heparan sulfate glycosylation is an inhibitor of heparan sulfate glycosylation. In further or alternative embodiments, the selective modulator of heparan sulfate sulfation is an inhibitor of heparan sulfate sulfation. In further or alternative embodiments, the selective modulator of heparan sulfate epimerization is an inhibitor of heparan sulfate epimerization.

In certain embodiments, any selective modulator of heparan sulfate used in any process described herein is a selective inhibitor of heparan sulfate. In specific embodiments, a selective inhibitor of heparan sulfate used in any process described herein is an inhibitor of 6-O sulfation. In some embodiments, a selective inhibitor of heparan sulfate used in any process described herein is a non-carbohydrate selective inhibitor of heparan sulfate.

In some embodiments, provided herein is a heparan sulfate proteoglycan comprising a core protein covalently linked to at least one heparan sulfate, wherein the at least one heparan sulfate comprises a plurality of glucosamine groups, and wherein less than 20% of the plurality of glucosamine groups are N-sulfated.

In certain embodiments, provided herein is a heparan sulfate proteoglycan comprising a core protein covalently linked to at least one heparan sulfate, wherein the at least one heparan sulfate comprises a plurality of glucosamine groups, and wherein less than 10% of the plurality of glucosamine groups are 6-OH sulfated.

In some embodiments, provided herein is a heparan sulfate proteoglycan comprising a core protein covalently linked to at least one heparan sulfate, wherein the at least one heparan sulfate comprises a plurality of glucosamine groups, and wherein less than 10% of the plurality of glucosamine groups are 2-OH sulfated.

Provided in certain embodiments herein is a compound having the structure:

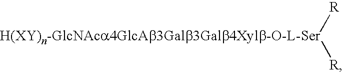

wherein:
a. each R is independently H or at least one amino acid,
b. n is 1-300;
c. each X is:

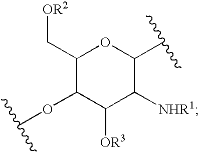

i. R¹ is H, COCH₃, or SO₃R⁵;
ii. R² is H, or SO₃R⁵;
iii. R³ is H, or SO₃R⁵;
d. each Y is:

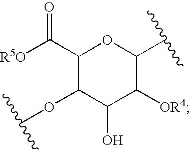

i. R⁴ is H, or SO₃R⁵;
ii. each R⁵ is independently selected from H and a negative charge;

or physiologically acceptable salts thereof,

and wherein the substituents have one or more of the following ratios:
R¹═SO₃R⁵ to R¹═COCH₃ is about 0:1 to about 0.2:1;
R²═SO₃R⁵ to R²═H is about 0:1 to about 0.1:1;
R⁴═SO₃R⁵ to R²═SO₃R⁵ is about 0:1 to about 0.7:1,
R⁴═SO₃R⁵ to R²═SO₃R⁵ is >1.05; or
R⁴═SO₃R⁵ to R⁴═H is about 0:1 to about 0.1:1.

In some embodiments, the ratio of R⁴═SO₃R⁵ to R²═SO₃R⁵ is about 0:1 to about 0.7:1. In certain embodiments, the ratio of R⁴═SO₃R⁵ to R²═SO₃R⁵ is about 0:1 to about 0.5:1. In some embodiments, the ratio of R⁴═SO₃R⁵ to R²═SO₃R⁵ is about 0:1 to about 0.4:1. In certain embodiments, the ratio of R⁴═SO₃R⁵ to R²═SO₃R⁵ is >1.05. In some embodiments, the ratio of R⁴═SO₃R⁵ to R²═SO₃R⁵ is >1.2. In certain embodiments, less than 10% of R⁴═SO₃R⁵. In some embodiments, each R is at least one amino acid. In certain embodiments, the compound is a human liver heparan sulfate proteoglycan.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates regions of heparan sulfate to which FGF-2 and VEGF bind.

FIG. 2 illustrates various selective heparan sulfate inhibitors.

FIG. 3 illustrates the inhibition of FGF-2 binding to heparan sulfate caused by 4-(4-(3,4-dimethoxyphenyl)-6-phenylpyrimidin-2-yl)morpholine at various concentrations.

FIG. 4 illustrates the modulation of heparan sulfate sulfation caused by 4-(4-(3,4-dimethoxyphenyl)-6-phenylpyrimidin-2-yl)morpholine at various concentrations. FIG. 4A illustrates the disaccharide modification of heparan sulfate caused by 4-(4-(3,4-dimethoxyphenyl)-6-phenylpyrimidin-2-yl)morpholine. FIG. 4B illustrates the modification of glucosamine sulfation (NS, 6S) and uronic acid sulfation (2S) caused by 4-(4-(3,4-dimethoxyphenyl)-6-phenylpyrimidin-2-yl)morpholine.

FIG. 5 illustrates the inhibition of FGF-2 binding to heparan sulfate caused by 7-((3-chlorophenyl)(pyridin-2-ylamino)methyl)-2-methylquinolin-8-ol at various concentrations.

FIG. 6 illustrates the modulation of heparan sulfate sulfation caused by 7-((3-chlorophenyl)(pyridin-2-ylamino)methyl)-2-methylquinolin-8-ol at various concentrations. FIG. 6A illustrates the disaccharide modification of heparan sulfate caused by 7-((3-chlorophenyl)(pyridin-2-ylamino)methyl)-2-methylquinolin-8-ol. FIG. 6B illustrates the modification of glucosamine sulfation (NS, 6S) and uronic acid sulfation (2S) caused by 7-((3-chlorophenyl)(pyridin-2-ylamino)methyl)-2-methylquinolin-8-ol.

FIG. 7 illustrates the inhibition of FGF-2 binding to heparan sulfate caused by 7-((2-fluorophenyl)(pyridin-2-ylamino)methyl)-2-methylquinolin-8-ol at various concentrations.

FIG. 8 illustrates the modulation of heparan sulfate sulfation caused by 7-((2-fluorophenyl)(pyridin-2-ylamino)methyl)-2-methylquinolin-8-ol at various concentrations. FIG. 8A illustrates the disaccharide modification of heparan sulfate caused by 7-((2-fluorophenyl)(pyridin-2-ylamino)methyl)-2-methylquinolin-8-ol. FIG. 8B illustrates the modification of glucosamine sulfation (NS, 6S) and uronic acid sulfation (2S) caused by 7-((2-fluorophenyl)(pyridin-2-ylamino)methyl)-2-methylquinolin-8-ol.

FIG. 9 illustrates the inhibition of FGF-2 binding to heparan sulfate caused by 7-((3-chlorophenyl)(acetylamino)methyl)-5-nitroquinolin-8-ol.

FIG. 10 illustrates the modulation of heparan sulfate sulfation caused by 7-((3-chlorophenyl)(acetylamino)methyl)-5-nitroquinolin-8-ol at various concentrations. FIG. 10A illustrates the disaccharide modification of heparan sulfate caused by 7-((3-chlorophenyl)(acetylamino)methyl)-5-nitroquinolin-8-ol. FIG. 10B illustrates the modification of glucosamine sulfation (NS, 6S) and uronic acid sulfation (2S) caused by 7-((3-chlorophenyl)(acetylamino)methyl)-5-nitroquinolin-8-ol.

FIG. 11 illustrates the inhibition of FGF-2 binding to heparan sulfate caused by 7-((thiophen-2-yl)(isobutoylamino)methyl)-5-nitroquinolin-8-ol at various concentrations.

FIG. 12 illustrates the modulation of heparan sulfate sulfation caused by 7-((thiophen-2-yl)(isobutoylamino)methyl)-5-nitroquinolin-8-ol at various concentrations. FIG. 12A illustrates the disaccharide modification of heparan sulfate caused by 7-((thiophen-2-yl)(isobutoylamino)methyl)-5-nitroquinolin-8-ol. FIG. 12B illustrates the modification of glucosamine sulfation (NS, 6S) and uronic acid sulfation (2S) caused by 7-((thiophen-2-yl)(isobutoylamino)methyl)-5-nitroquinolin-8-ol.

FIG. 13 illustrates the inhibition of FGF-2 binding to heparan sulfate caused by 4-ethyl-7-(4-nitro-2-(trifluoromethyl)phenoxy)-2H-chromen-2-one at various concentrations.

FIG. 14 illustrates the modulation of heparan sulfate sulfation caused by 4-ethyl-7-(4-nitro-2-(trifluoromethyl)phenoxy)-2H-chromen-2-one at various concentrations. FIG. 14A illustrates the disaccharide modification of heparan sulfate caused by 4-ethyl-7-(4-nitro-2-(trifluoromethyl)phenoxy)-2H-chromen-2-one. FIG. 14B illustrates the modification of glucosamine sulfation (NS, 6S) and uronic acid sulfation (2S) caused by 4-ethyl-7-(4-nitro-2-(trifluoromethyl)phenoxy)-2H-chromen-2-one.

FIGS. 15A-C illustrates the reduction of GAG accumulation in in vitro models of MPS I, II, and IIIA with sodium chlorate.

FIGS. 16A-D illustrate the inhibition of heparan sulfate biosynthesis in human MPS IIIA fibroblasts with compounds 1, 5, 7, and 8, respectively.

FIG. 17 illustrates the inhibition of FGF-2 binding to heparan sulfate caused by compound 7 at various concentrations.

FIG. 18 illustrates the modulation of heparan sulfate sulfation caused by compound 7 at various concentrations. FIG. 18A illustrates the disaccharide modification of heparan sulfate caused by compound 7. FIG. 18B illustrates the modification of glucosamine sulfation (NS, 6S) and uronic acid sulfation (2S) caused compound 7.

FIG. 19 illustrates the inhibition of FGF-2 binding to heparan sulfate caused by compound 8 at various concentrations.

FIG. 20 illustrates the modulation of heparan sulfate sulfation caused by compound 8 at various concentrations. FIG. 20A illustrates the disaccharide modification of heparan sulfate caused by compound 8. FIG. 20B illustrates the modification of glucosamine sulfation (NS, 6S) and uronic acid sulfation (2S) caused compound 8.

FIG. 21 illustrates efficacy of heparan sulfate inhibitors in treating ovarian cancer. FIG. 21A illustrates the modulation of heparan sulfate sulfation caused by compound 9 at various concentrations. FIG. 21B illustrates the effect of compound 9 on ovarian cancer cell lines. FIG. 21C illustrates the modulation of heparan sulfate sulfation in the liver of a mouse in which an ovarian cancer tumor has been grown.

### DETAILED DESCRIPTION OF THE INVENTION

### Heparan Sulfate Modulators

Provided in certain embodiments herein are heparan sulfate modulators, and in specific embodiments heparan sulfate inhibitors. In some embodiments, heparan sulfate modulators include heparan sulfate inhibitors and/or heparan sulfate promoters. In general, heparan sulfate modulators modulate or alter the nature (e.g., character, structure, and/or concentration) of heparan sulfate (e.g., the endogenous heparan sulfate on a protein or biomolecule, or in a cell, tissue, organ and/or individual). Heparan sulfate inhibitors modulate or alter the nature (e.g., character, structure, and/or decreased concentration) of heparan sulfate (e.g., the endogenous heparan sulfate on a protein or biomolecule, or in a cell, tissue, organ or individual). In some embodiments, heparan sulfate inhibitors are used, e.g., in therapies to reduce heparan sulfate accumulation in a cell, or individual (e.g., substrate accumulation therapy), by administering an effective or therapeutically effective amount of a heparan sulfate inhibitor to a cell, or individual, in need thereof. Heparan sulfate (HS) is a glycan (specifically, a glycosaminoglycan (GAG)) comprising a plurality of disaccharide units. One or more of the disaccharide units of heparan sulfate comprise a glucosamine (GlcN) (Formula I) group linked to an uronic acid group (Formula II). Uronic acid (UA) groups include glucuronic acid (GlcA) groups and the epimers thereof (i.e., iduronic acid (IdoA) groups). Each unit (e.g., glucosamine or uronic acid group) is optionally and independently sulfated. Within the class of compounds described as heparan sulfate, there is broad variability with respect to the location and degree of sulfation and other modifications. Therefore, in various instances, glucuronic acid is sometimes O-sulfated at the C2 position (GlcA(2S)); iduronic acid is sometimes O-sulfated at the C2 position (IdoA(2S)); glucosamine is sometimes unmodified, glucosamine is sometimes acylated at the N position (GlcNAc); glucosamine is sometimes sulfated at the N-position (GlcNS); glucosamine is sometimes O-sulfated at the C6 position (GlcNAc(6S)); glucosamine is sometimes O-sulfated at the C3 position (GlcNAc(3S)); glucosamine is sometimes O-sulfated at the C6 position and the N-position (GlcNS(6S)); and the like. In certain instances, the disaccharide units are connected to a core protein via and/or comprising a linkage tetrasaccharide, which generally has the structure -GlcAβ3Galβ3Galβ4Xylβ-O—. The linkage tetrasaccharide can be modified by 2-O-phosphorylation of the xylose and 6-O sulfation of either of the galactose residues, in any combination. In some instances, the disaccharide units are connected to a core protein at an L-serine amino acid group (e.g., HS-GlcAβ3Galβ3Galβ4Xylβ-O-L-Ser).

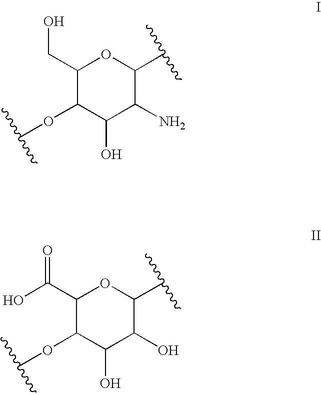

In some embodiments, heparan sulfate modulators, and in specific embodiments heparan sulfate inhibitors, described herein modulate heparan sulfate biosynthesis, e.g., heparan sulfate glycosylation, heparan sulfate sulfation (N or O sulfation), heparan sulfate phosphorylation, and/or heparan sulfate epimerization. As utilized herein, modulation of heparan sulfate biosynthesis or the modulation of heparan sulfate glycosylation, heparan sulfate sulfation, or heparan sulfate epimerization includes the promotion of one or more of and/or the inhibition of one or more of heparan sulfate glycosylation, heparan sulfate sulfation, heparan sulfate phosphorylation, or heparan sulfate epimerization.

The modulation of heparan sulfate glycosylation includes the modulation of the production of the linkage region that connects heparan sulfate to a core protein (e.g., GlcAβ3Galβ3Galβ4Xylβ-O—). In certain embodiments, the modulation of the production of the linkage region includes the inhibition of the production of or synthesis of the linkage region. In some embodiments, the modulation of the production of the linkage region includes the cleavage of one or more bonds within the linkage region. In certain instances, a heparan sulfate inhibitor described herein directly promotes production or cleavage, while in other instances, a heparan sulfate inhibitor impacts (including modifying the character of) an endogenous chemical (e.g., by activating or deactivating an enzyme) that inhibits production or promotes cleavage of the linkage region. In some embodiments, an inhibitor of heparan sulfate that modulates the production of the linkage region inhibits one or more glycosyltransferase. In some embodiments, the glycosyltransferase is xylosyltransfarase (e.g., xylosyltransfarase I and/or II), galactosyltransferase (e.g., galactosyltransferase I and/or II), glucuronosyltransferase (e.g., glucuronosyltransferase I), or a combination thereof. In specific embodiments, the glycosyltransferase is xylosyltransfarase (e.g., xylosyltransfarase I and/or II). In specific embodiments, the glycosyltransferase is galactosyltransferase (e.g., galactosyltransferase I and/or II). In specific embodiments, the glycosyltransferase is glucuronosyltransferase (e.g., glucuronosyltransferase I). In some embodiments, the glycosyltransferase is a uronic acid glycosyl transferase. In more specific embodiments, the glycosyltransferase is a specific uronic acid glycosyl transferase as compared to amino sugar transferases (e.g., GlcNAc transferases). In some embodiments, the glycosyltransferase is an amino sugar transferase. In more specific embodiments, the glycosyltransferase is a specific amino sugar transferase as compared to uronic acid glycosyl transferases (e.g., GlcA/IdoA transferases). In certain instances, specificity includes inhibition of the indicated type of glycosyltransferase by a ratio of greater than 10:1, greater than 9:1, greater than 8:1, greater than 7:1, greater than 6:1, greater than 5:1, greater than 4:1, greater than 3:1, or greater than 2:1 over the other types of glycosyltransferase.

The modulation of heparan sulfate glycosylation further includes the modulation of the initiation of heparan sulfate synthesis on the linkage region that connects heparan sulfate to a core protein (e.g., GlcAβ3Galβ3Galβ4Xylβ-O—). In certain embodiments, the modulation of the initiation of heparan sulfate synthesis on the linkage region includes the inhibition of the production of or synthesis or modification of the linkage region. In some embodiments, the modulation of the initiation of heparan sulfate synthesis to the linkage region includes the cleavage of a bond connecting the first glucosamine group to the linkage region. In certain instances, a heparan sulfate inhibitor described herein directly promotes synthesis or cleavage, while in other instances, a heparan sulfate inhibitor impacts an endogenous chemical (e.g., by activating or deactivating an enzyme) that inhibits synthesis or promotes cleavage of a bond connecting the first glucosamine group to the linkage region. In some embodiments, an inhibitor of heparan sulfate that modulates the initiation of heparan sulfate synthesis to the linkage region inhibits one or more glycosyltransferase, e.g., N-acetylglucosamine transferase (e.g., N-acetylglucosamine transferase I).

The modulation of heparan sulfate glycosylation further includes the modulation of the synthesis (i.e., polymerization) of heparan sulfate. In certain embodiments, the modulation of the synthesis of heparan sulfate includes the inhibition of synthesis of the heparan sulfate and/or cleavage of a heparan sulfate bond. In certain instances, a heparan sulfate inhibitor described herein directly promotes synthesis or cleavage, while in other instances, a heparan sulfate inhibitor impacts an endogenous chemical (e.g., by activating or deactivating an enzyme) that inhibits synthesis or promotes cleavage of a heparan sulfate bond. In some embodiments, an inhibitor of heparan sulfate that modulates the synthesis of heparan sulfate inhibits one or more glycosyltransferase, e.g., glucuronosyltransferase (e.g., glucuronosyltransferase II), N-acetylglucosamine transferase (e.g., N-acetylglucosamine transferase II), or a combination thereof. In specific embodiments, an inhibitor of heparan sulfate inhibits glucuronosyltransferase (e.g., glucuronosyltransferase II). In some specific embodiments, an inhibitor of heparan sulfate inhibits N-acetylglucosamine transferase (e.g., N-acetylglucosamine transferase II).

The modulation of heparan sulfate sulfation includes the modulation of the oxygen sulfation (i.e., sulfation of the hydroxy group used interchangeably herein with O-sulfation), nitrogen sulfation (i.e., sulfation of the amino group and used interchangeably herein with N-sulfation), or a combination thereof. In some embodiments, a heparan sulfate inhibitor modulates one or more sulfotransferase. In some embodiments, modulation of O-sulfation includes the inhibition of the 2-O sulfation of an uronic acid group of the heparan sulfate (used interchangeably herein with a uronic acid moiety), the 3-O sulfation of a glucosamine group of the heparan sulfate (used interchangeably herein with a glucosamine moiety), the 6-O sulfation of a glucosamine group of the heparan sulfate, or a combination thereof. In some embodiments, heparan sulfate inhibitors described herein inhibit of 2-O sulfation of an uronic acid group of the heparan sulfate. In certain embodiments, heparan sulfate inhibitors described herein inhibit 3-O sulfation of a glucosamine group of the heparan sulfate. In some embodiments, heparan sulfate inhibitors described herein inhibit 6-O sulfation of a glucosamine group of the heparan sulfate. Furthermore, in some embodiments, modulation of O-sulfation includes the promotion of the 2-O sulfation of an uronic acid group of the heparan sulfate, the 3-O sulfation of a glucosamine group of the heparan sulfate, the 6-O sulfation of a glucosamine group of the heparan sulfate, or a combination thereof. In certain instances, a single heparan sulfate inhibitor inhibits one type of sulfation while also promoting another. For example, in various embodiments, a single heparan sulfate inhibitor promotes N-sulfation while inhibiting 2-O sulfation, a single heparan sulfate inhibitor promotes N-sulfation while inhibiting 6-O sulfation, a single heparan sulfate inhibitor promotes N-sulfation while inhibiting 3-O sulfation, a single heparan sulfate inhibitor promotes 2-O sulfation while inhibiting N-sulfation, a single heparan sulfate inhibitor promotes 6-O sulfation while inhibiting N-sulfation, a single heparan sulfate inhibitor promotes 2-O sulfation while inhibiting 6-O sulfation, a single heparan sulfate inhibitor promotes 6-O sulfation while inhibiting 2-O sulfation, a single heparan sulfate inhibitor promotes 6-O and 2-O sulfation while inhibiting N-sulfation, or the like. In certain embodiments, the heparan sulfate inhibitor specifically inhibits, modulates or promotes 2-O sulfation of uronic acid groups, the heparan sulfate inhibitor specifically inhibits, modulates or promotes 3-O sulfation of glucosamine groups, the heparan sulfate inhibitor specifically inhibits, modulates or promotes 6-O sulfation of glucosamine groups, the heparan sulfate inhibitor specifically inhibits, modulates or promotes N-sulfation of glucosamine groups, the heparan sulfate inhibitor specifically inhibits, or modulates or promotes acetylation of amino groups of glucosamine groups. In certain instances, specificity includes inhibition, modulation or promotion of the indicated type of sulfation by a ratio of greater than 10:1, greater than 9:1, greater than 8:1, greater than 7:1, greater than 6:1, greater than 5:1, greater than 4:1, greater than 3:1, or greater than 2:1 over the other types of sulfation.

In some embodiments, the modulation of heparan sulfate epimerization includes the inhibition of or the promotion of epimerization from glucuronic acid to iduronic acid. In certain embodiments, the modulation of heparan sulfate epimerization includes the inhibition of or the promotion of epimerization from iduronic acid to glucuronic acid. In some embodiments, the modulation of heparan sulfate epimerization includes the inhibition or activation of a heparan sulfate epimerase.

Furthermore, in certain embodiments, a heparan sulfate inhibitor includes an agent that inhibits acetylation or deacetylation of heparan sulfate amino groups (e.g., on the glucosamine groups contained therein). Heparan sulfate inhibitors described herein also include agents that promote the acetylation or deacetylation of heparan sulfate amino groups (e.g., on the glucosamine groups contained herein).

In certain embodiments, heparan sulfate inhibitors or modulators of heparan sulfate biosynthesis are compounds that modify the nature (e.g., character, structure and/or concentration) of heparan sulfate endogenous to a cellular compartment (including vesicles), cell, tissue, organ or individual when contacted or administered to the cell, tissue, organ or individual. It is to be understood that contacting a cell, tissue, or organ is possible via the administration to an individual within whom such cell, tissue or organ resides. In certain instances, heparan sulfate inhibitors or modulators of heparan sulfate biosynthesis modify the character and/or concentration of heparan sulfate in a targeted type of cell, tissue type or organ. In other instances, heparan sulfate inhibitors or modulators of heparan sulfate biosynthesis modify the character and/or concentration of heparan sulfate in a systemic manner.

In certain embodiments, a heparan sulfate inhibitor (used interchangeably herein with a modulator of heparan sulfate biosynthesis) alters or disrupts the nature of heparan sulfate compared to endogenous heparan sulfate in an amount sufficient to alter or disrupt heparan sulfate binding, heparan sulfate signaling, or a combination thereof. In some embodiments, the heparan sulfate inhibitor alters or disrupts the nature of heparan sulfate in a selected tissue type or organ compared to endogenous heparan sulfate in the selected tissue type or organ. In some embodiments, the selected tissue is, by way of non-limiting example, brain tissue, liver tissue, kidney tissue, intestinal tissue, skin tissue, or the like. In some embodiments, a heparan sulfate inhibitor as described herein alters or disrupts the nature of heparan sulfate compared to endogenous heparan sulfate by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, or more. In specific embodiments, the selected tissue is brain tissue. In other specific embodiments, the selected tissue is liver tissue. In other specific embodiments, the selected tissue is kidney tissue. In other specific embodiments, the selected tissue is intestinal tissue. In other specific embodiments, the selected tissue is skin tissue. In certain embodiments, the heparan sulfate inhibitor described herein alters or disrupts the concentration of heparan sulfate compared to endogenous heparan sulfate in a cell, tissue, organ, or individual by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, or more. In certain embodiments, the heparan sulfate inhibitor described herein alters or disrupts (e.g., reduces the amount of) the acetylation, sulfation, O-sulfation, the 2-O sulfation, the 3-O sulfation, the 6-O sulfation, or the N-sulfation of heparan sulfate compared to endogenous heparan sulfate in a cell, tissue, organ, or individual by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, or more. In specific embodiments, the heparan sulfate inhibitor described herein reduces the amount of acetylation of heparan sulfate (e.g., compared to endogenous heparan sulfate in an untreated cell, tissue, organ, or individual). In specific embodiments, the heparan sulfate inhibitor described herein reduces the amount of sulfation (e.g., compared to endogenous heparan sulfate in an untreated cell, tissue, organ, or individual). In other specific embodiments, the heparan sulfate inhibitor described herein reduces the amount of O-sulfation (e.g., compared to endogenous heparan sulfate in an untreated cell, tissue, organ, or individual). In other specific embodiments, the heparan sulfate inhibitor described herein reduces the amount of 2-O sulfation (e.g., compared to endogenous heparan sulfate in an untreated cell, tissue, organ, or individual). In other specific embodiments, the heparan sulfate inhibitor described herein reduces the amount of 3-O sulfation (e.g., compared to endogenous heparan sulfate in an untreated cell, tissue, organ, or individual). In other specific embodiments, the heparan sulfate inhibitor described herein reduces the amount of 6-O sulfation (e.g., compared to endogenous heparan sulfate in an untreated cell, tissue, organ, or individual). In other specific embodiments, the heparan sulfate inhibitor described herein reduces the amount of N-sulfation (e.g., compared to endogenous heparan sulfate in an untreated cell, tissue, organ, or individual). In certain embodiments, the heparan sulfate inhibitor described herein alters or disrupts (e.g., reduces) the chain length (or heparan sulfate molecular weight) of heparan sulfate (e.g., compared to endogenous heparan sulfate in an untreated cell, tissue, organ, or individual) by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, or more. In certain embodiments, the heparan sulfate inhibitor described herein alters or disrupts (e.g., compared to endogenous heparan sulfate in an untreated cell, tissue, organ, or individual), in combination (e.g., the sum of the change in amount of sulfation, concentration, and chain length), the nature of heparan sulfate (e.g., compared to endogenous heparan sulfate in an untreated cell, tissue, organ, or individual) by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, or more. In certain embodiments, a heparan sulfate inhibitor as described herein alters or disrupts (e.g., reduces) the sulfation and/or phosphorylation of the linkage region of heparan sulfate (e.g., compared to endogenous heparan sulfate in an untreated organism, organ, tissue or cell) by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, or more. As used herein, endogenous heparan sulfate is described as heparan sulfate present in the absence of treatment or contact with a heparan sulfate inhibitor. In some embodiments, the comparison between altered or disrupted heparan sulfate compared to endogenous heparan sulfate is based on the average characteristic (e.g., the concentration, N-acetylation, sulfation, O-sulfation, 2-O sulfation, 3-O sulfation, 6-O sulfation, 2-O phosphorylation, N-sulfation, chain length or molecular weight, combinations thereof, or the like) of the altered or disrupted heparan sulfate. Furthermore, in some embodiments, the comparison between altered or disrupted heparan sulfate is based on a comparison of the sulfated and/or non-sulfated domains of the modified heparan sulfate to the sulfated and/or non-sulfated domains endogenous heparan sulfate. In some instances, the degree or nature of sulfation in the domains that have high sulfation in endogenous heparan sulfate are increased or decreased in the modified heparan sulfate. Similarly, in certain instances, the degree or nature of sulfation in the domains that have low sulfation in endogenous heparan sulfate have increased sulfation in the modified heparan sulfate. In some instances, domain organization can be determined using enzymes that cleave only in N-sulfated domains (e.g., Heparin Lyase I) or enzymes that cleave only in N-acetylated domains (e.g., heparin lyase III or K5 lyase). The concentration, amount, character, and/or structure of heparan sulfate can be determined in any suitable manner, including those set forth herein. As used herein, altering includes increasing or decreasing. Furthermore, as used herein, disrupting includes reducing or inhibiting.

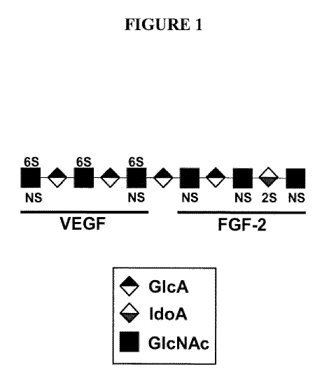

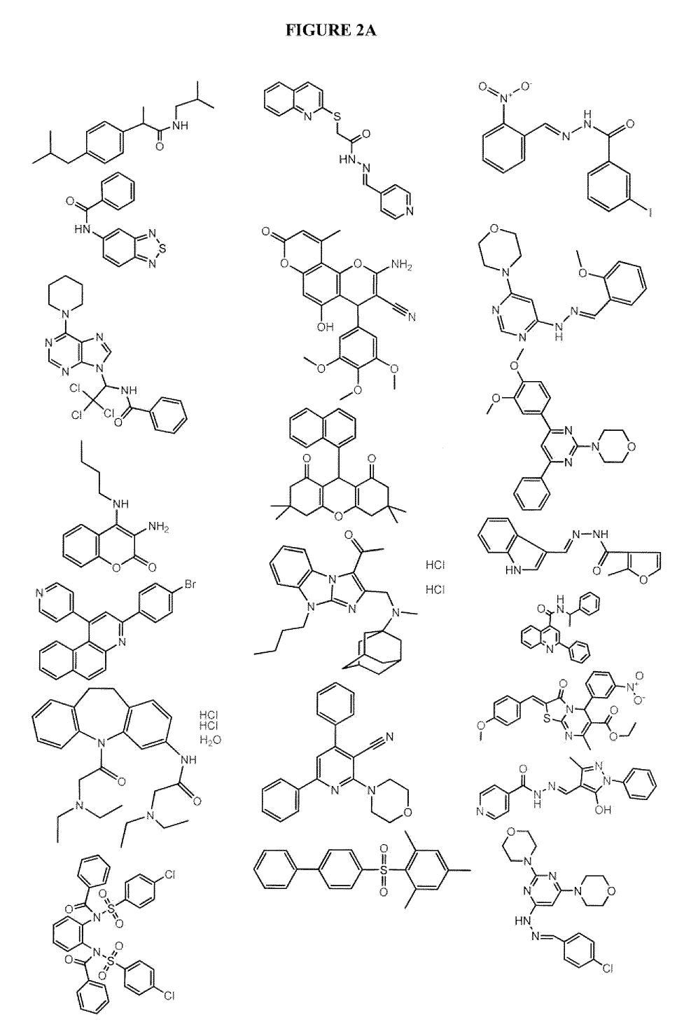

In specific embodiments, the heparan sulfate inhibitor described herein alters or disrupts the nature of the heparan sulfate such that it inhibits heparan sulfate signaling. In other specific embodiments, the heparan sulfate inhibitor described herein alters or disrupts the nature of the heparan sulfate such that it inhibits heparan sulfate binding. In more specific embodiments, the heparan sulfate inhibitor described herein alters or disrupts the nature of the heparan sulfate such that it inhibits heparan sulfate binding and heparan sulfate signaling. In some embodiments, the heparan sulfate inhibitor alters or disrupts the nature of the heparan sulfate such that it inhibits the binding, signaling, or a combination thereof of any lectin (including polypeptides) subject to heparan sulfate binding, signaling or a combination thereof, in the absence of a heparan sulfate inhibitor. In some embodiments, the polypeptide is, by way of non-limiting example, a growth factor. In specific embodiments, the growth factor is, by way of non-limiting example, fibroblast growth factor (FGF) or vascular endothelia growth factor (VEGF). In more specific embodiments, the heparan sulfate lectin is FGF. In other specific embodiments, the heparan sulfate lectin is VEGF. In certain instances, FGF and VEGF bind to heparan sulfate in regions as set forth in FIG. 1.

In some embodiments, a heparan sulfate inhibitor is an agent that when contacted or administered to a human liver cell, a human liver tissue, a human liver, or a human results in an average heparan sulfate sulfation of less than 1.2, less than 1.1, less than 1.0, less than 0.9, less than 0.8, less than 0.7, less than 0.6, or less than 0.5 in the liver cell, liver tissue, the liver, or the liver of the human, respectively. As used herein, the average heparan sulfate sulfation refers to number of sulfate substituents on each disaccharide component (e.g., each GlcA-GlcN or each IdoA-GlcN group) of the heparan sulfate. In some embodiments, a heparan sulfate inhibitor is an agent that when contacted or administered to a pig liver cell, pig liver tissue, a pig liver, or a pig results in an average heparan sulfate sulfation of less than 1.0, less than 0.9, less than 0.8, less than 0.7, less than 0.6, or less than 0.5 in the liver cell, liver tissue, the liver, or the liver of the pig, respectively. In some embodiments, a heparan sulfate inhibitor is an agent that when contacted or administered to a mouse liver cell, mouse liver tissue, a mouse liver, or a mouse results in an average heparan sulfate sulfation of less than 0.9, less than 0.8, less than 0.7, less than 0.6, less than 0.5, less than 0.4, or less than 0.3 in the liver cell, liver tissue, the liver, or the liver of the mouse, respectively.

In some embodiments, a heparan sulfate inhibitor is an agent that when contacted or administered to a human liver cell, a human liver tissue, a human liver, or a human results in an average heparan sulfate 2S sulfation of each disaccharide component (absent any other sulfation of the disaccharide component, i.e., UA2S-GlcNAc) of less than 1.2 mol. %, less than 1.1 mol. %, less than 1.0 mol. %, less than 0.9 mol. %, less than 0.8 mol. %, less than 0.7 mol. %, less than 0.6 mol. %, or less than 0.5 mol. % in the liver cell, liver tissue, the liver, or the liver of the human, respectively. As used herein, unless otherwise indicated, 2S sulfation includes sulfation of any uronic acid group (e.g., IdoA or GlcA). Furthermore, as used herein, mol. % is the molar percentage of the selected disaccharide component compared to the total number of disaccharide components in the heparan sulfate(s) present and/or analyzed. In some embodiments, a heparan sulfate inhibitor is an agent that when contacted or administered to a human liver cell, a human liver tissue, a human liver, or a human results in an average heparan sulfate N-sulfation of each disaccharide component (absent any other sulfation of the disaccharide component, i.e., UA-GlcNS) of less than 15 mol. %, less than 14 mol. %, less than 12 mol. %, less than 10 mol. %, less than 8 mol. %, less than 7 mol. %, less than 6 mol. %, or less than 5 mol. % in the liver cell, liver tissue, the liver, or the liver of the human, respectively. In some embodiments, a heparan sulfate inhibitor is an agent that when contacted or administered to a human liver cell, a human liver tissue, a human liver, or a human results in an average heparan sulfate NS and 6S sulfation of each disaccharide component (absent any other sulfation of the disaccharide component, i.e., UA-GlcNS6S) of less than 7 mol. %, less than 6 mol. %, less than 5 mol. %, less than 4 mol. %, less than 3 mol. % in the liver cell, liver tissue, the liver, or the liver of the human, respectively. In some embodiments, a heparan sulfate inhibitor is an agent that when contacted or administered to a human liver cell, a human liver tissue, a human liver, or a human results in an average heparan sulfate 6S sulfation of each disaccharide component (absent any other sulfation of the disaccharide component, i.e., UA-GlcNAc6S) of less than 10 mol. %, less than 8 mol. %, less than 7 mol. %, less than 6 mol. %, less than 5 mol. %, less than 4 mol. %, less than 3 mol. % in the liver cell, liver tissue, the liver, or the liver of the human, respectively. In some embodiments, a heparan sulfate inhibitor is an agent that when contacted or administered to a human liver cell, a human liver tissue, a human liver, or a human results in an average heparan sulfate 2S and NS sulfation of each disaccharide component (absent any other sulfation of the disaccharide component, i.e., UA2S-GlcNS) of less than 6 mol. %, less than 5 mol. %, less than 4 mol. %, less than 3 mol. % in the liver cell, liver tissue, the liver, or the liver of the human, respectively. In some embodiments, a heparan sulfate inhibitor is an agent that when contacted or administered to a human liver cell, a human liver tissue, a human liver, or a human results in an average heparan sulfate 2S and 6S sulfation of each disaccharide component (absent any other sulfation of the disaccharide component, i.e., UA2S-GlcNAc6S) of less than 0.7 mol. %, less than 0.6 mol. %, less than 0.5 mol. %, less than 0.4 mol. %, or less than 0.3 mol. % in the liver cell, liver tissue, the liver, or the liver of the human, respectively. In some embodiments, a heparan sulfate inhibitor is an agent that when contacted or administered to a human liver cell, a human liver tissue, a human liver, or a human results in an average heparan sulfate 2S, NS and 6S sulfation of each disaccharide component (absent any other sulfation of the disaccharide component, i.e., UA2S-GlcNS6S) of less than 20 mol. %, less than 18 mol. %, less than 16 mol. %, less than 14 mol. %, or less than 12 mol. % in the liver cell, liver tissue, the liver, or the liver of the human, respectively. In some embodiments, a heparan sulfate inhibitor described herein reduces the amount (compared to levels found in endogenous HS) of UA2S-GlcNAc, UA-GlcNS, UA-GlcNS6S, UA-GlcNAc6S, UA2S-GlcNS, UA2S-GlcNAc6S, UA2S-GlcNS6S, or combinations thereof. In certain embodiments, the amount of heparan sulfate inhibitor administered is an effective amount. In further embodiments, the effective amount is an amount having a minimal lethality. In more specific embodiments, the LD50:ED50 is greater than 1.1, greater than 1.2, greater than 1.3, greater than 1.4, greater than 1.5, greater than 2, greater than 5, greater than 10, or more. In some embodiments, a therapeutically effective amount is about 0.1 mg to about 10 g.

In some embodiments, a heparan sulfate modulator, and in specific embodiments heparan sulfate inhibitor, described herein is a selective heparan sulfate inhibitor. In some embodiments, the selective heparan sulfate inhibitor selective alters or disrupts the nature (e.g., concentration, chain length, sulfation, etc.) of heparan sulfate compared to other glycans. In certain embodiments, the selective heparan sulfate inhibitor selective affects the biosynthesis of glycosaminoglycans (GAGs), such as heparan sulfate, chondroitin sulfate, dermatan sulfate, keratin sulfate, and/or hyaluronan, but not extracellular glycans (e.g., N-linked, O-linked, lipid linked, or the like). In certain embodiments, selective heparan sulfate inhibitors selectively inhibit GAGs compared to extracellular glycans by a ratio of greater than 2:1, 3:1, 4:1, 5:1, 6:1, 8:1, 10:1 or more.

In certain embodiments, provided herein is a selective inhibitor of heparan sulfate biosynthesis. In some embodiments, selective inhibitors of heparan sulfate biosynthesis selectively inhibit heparan sulfate biosynthesis, but do not significantly affect the biosynthesis of N-linked glycans. In some embodiments, selective inhibitors of heparan sulfate biosynthesis selectively inhibit heparan sulfate biosynthesis, but do not significantly affect the biosynthesis of O-linked glycans. In some embodiments, selective inhibitors of heparan sulfate biosynthesis selectively inhibit heparan sulfate biosynthesis, but do not significantly affect the biosynthesis of gangliosides. In some embodiments, selective inhibitors of heparan sulfate biosynthesis selectively inhibit heparan sulfate biosynthesis, but do not significantly affect the biosynthesis of chondroitin sulfate. In some embodiments, selective inhibitors of heparan sulfate biosynthesis selectively inhibit heparan sulfate biosynthesis, but do not significantly affect the biosynthesis of dermatan sulfate. In some embodiments, selective inhibitors of heparan sulfate biosynthesis selectively inhibit heparan sulfate biosynthesis, but do not significantly affect the biosynthesis of keratin. In some embodiments, selective inhibitors of heparan sulfate biosynthesis selectively inhibit heparan sulfate biosynthesis, but do not significantly affect the biosynthesis of N-linked glycans, do not significantly affect the biosynthesis of O-linked glycans, and do not significantly affect the biosynthesis of gangliosides. In certain embodiments, selective heparan sulfate inhibitors selectively inhibit heparan sulfate over the glycans over which the inhibitor is selective by a ratio of greater than 2:1, 3:1, 4:1, 5:1, 6:1, 8:1, 10:1 or more. In some embodiments, the selective inhibitor of heparan sulfate biosynthesis inhibits heparan sulfate by an amount that is greater than 4 standards of deviation from the mean untreated amount (e.g., in a cell that expresses heparan sulfate), and does not substantially inhibit the biosynthesis of another glycan (e.g., O-linked glycan, N-linked glycan and/or ganglioside). In specific embodiments, a compound does not inhibit the biosynthesis if the biosynthesis of that glycan is inhibited by an amount that is less than 2 standards of deviation from the mean untreated amount (e.g., in a cell that expresses the glycan).

In some embodiments, inhibition of the various glycans can be determined in any suitable manner. For example, in some embodiments, inhibition of a glycan is determined by the reduced amount of lectin that binds the glycan in a cell after the cell has been treated with a selective modulator, as compared to the amount of lectin that binds the glycan in the cell prior to being treated with the selective modulator. In some embodiments, a selective modulator or inhibitor of heparan sulfate selectively inhibits glycosaminoglycan (e.g., heparan sulfate) lectin (e.g., FGF) binding of a glycosaminoglycan (e.g., heparan sulfate) in a cell, when the cell is contacted with the selective modulator of heparan sulfate, relative to extracellular glycan (e.g., N-linked glycan) lectin (e.g., PHA) binding of a extracellular glycan (e.g., N-linked glycan) in a cell, when the cell is contacted with the selective modulator or inhibitor of heparan sulfate. In some embodiments, a selective modulator or inhibitor of heparan sulfate selectively inhibits FGF binding in a cell, when the cell is contacted with the selective modulator of heparan sulfate, relative to Phaseolus Vulgaris (PHA) binding in a cell, when the cell is contacted with the selective modulator of heparan sulfate. In certain embodiments, selective heparan sulfate inhibitor selectively inhibit sulfated FGF binding in a cell compared to PHA binding in a cell by a ratio of greater than 2:1, 3:1, 4:1, 5:1, 6:1, 8:1, 10:1 or more. Binding ratios can be determined in any suitable manner including, e.g., as a comparison of percent binding inhibited compared to a sample that has not been treated with the selective modulator of heparan sulfate.

In some embodiments, the selective heparan sulfate modulator, and in specific embodiments heparan sulfate inhibitor, selectively affects the biosynthesis of sulfated GAGs, but not non-sulfated GAGs (e.g., hyaluronan) or extracellular glycans. In certain embodiments, selective heparan sulfate inhibitors selectively inhibit sulfated GAGs compared to non-sulfated GAGs and extracellular glycans by a ratio of greater than 2:1, 3:1, 4:1, 5:1, 6:1, 8:1, 10:1 or more. In specific embodiments, inhibition is greater than 2:1 (sulfated GAG:non-sulfated GAG). In some specific embodiments, inhibition is greater than 3:1 (sulfated GAG:non-sulfated GAG). In some specific embodiments, inhibition is greater than 4:1 (sulfated GAG:non-sulfated GAG). In some specific embodiments, inhibition is greater than 5:1 (sulfated GAG:non-sulfated GAG). In some specific embodiments, inhibition is greater than 6:1 (sulfated GAG:non-sulfated GAG). In some specific embodiments, inhibition is greater than 8:1 (sulfated GAG:non-sulfated GAG). In some specific embodiments, inhibition is greater than 10:1 (sulfated GAG:non-sulfated GAG). In some embodiments, selective heparan sulfate inhibitors selectively inhibit the biosynthesis of heparan sulfate, chondroitin sulfate and dermatan sulfate, but not keratin sulfate, non-sulfated GAGs or extracellular glycans. In certain embodiments, selective heparan sulfate inhibitors selectively inhibit heparan sulfate, chondroitin sulfate and dermatan sulfate, compared to keratin sulfate, non-sulfated GAGs and extracellular glycans by a ratio of greater than 2:1, 3:1, 4:1, 5:1, 6:1, 8:1, 10:1 or more. In some embodiments, selective heparan sulfate inhibitors selective inhibit heparan sulfate, but not other glycans (e.g., other GAGs and extracellular glycans). In specific embodiments, inhibition is greater than 2:1 (HS:other glycans). In some specific embodiments, inhibition is greater than 3:1 (HS:other glycans). In some specific embodiments, inhibition is greater than 4:1 (HS:other glycans). In some specific embodiments, inhibition is greater than 5:1 (HS:other glycans). In some specific embodiments, inhibition is greater than 6:1 (HS:other glycans). In some specific embodiments, inhibition is greater than 8:1 (HS:other glycans). In some specific embodiments, inhibition is greater than 10:1 (HS:other glycans). In certain embodiments, selective heparan sulfate inhibitors selectively inhibit heparan sulfate compared to other GAGs and extracellular glycans by a ratio of greater than 2:1, 3:1, 4:1, 5:1, 6:1, 8:1, 10:1 or more. In some embodiments, inhibition is greater than 2:1 (HS:N-linked glycans). In some specific embodiments, inhibition is greater than 3:1 (HS:N-linked glycan). In some specific embodiments, inhibition is greater than 4:1 (HS:N-linked glycans). In some specific embodiments, inhibition is greater than 5:1 (HS:N-linked glycans). In some specific embodiments, inhibition is greater than 6:1 (HS:N-linked glycans). In some specific embodiments, inhibition is greater than 8:1 (HS:N-linked glycans). In some specific embodiments, inhibition is greater than 10:1 (HS:N-linked glycans).

Furthermore, in certain embodiments, heparan sulfate inhibitors selectively modulate specific types of action that inhibit heparan sulfate function. For example, in some embodiments, heparan sulfate inhibitors selectively modulate sulfation, glycosylation, phosphorylation, or epimerization. In some embodiments, certain heparan sulfate modulators, and in specific embodiments heparan sulfate inhibitors, selectively modulate (e.g., promote or inhibit) 2-O sulfation over other types of sulfation (e.g., NS, 6-O, or 3-O). In some embodiments, certain heparan sulfate modulators, and in specific embodiments heparan sulfate inhibitors, selectively modulate (e.g., promote or inhibit) 6-O sulfation. In some embodiments, certain heparan sulfate modulators, and in specific embodiments heparan sulfate inhibitors, selectively modulate (e.g., promote or inhibit) N-sulfation. In some embodiments, certain heparan sulfate modulators, and in specific embodiments heparan sulfate inhibitors, selectively modulate (e.g., promote or inhibit) 2-O phosphorylation.

In some embodiments, certain heparan sulfate modulators, and in specific embodiments heparan sulfate inhibitors, selectively modulate (e.g., promote or inhibit) glycosyltransferase, and/or specific types of glycosyltransferase. In some embodiments, heparan sulfate modulators, and in specific embodiments heparan sulfate inhibitors, selectively modulate (e.g., promote or inhibit) one of a xylosyltransfarase, a galactosyltransferase, a glucuronosyltransferase, or a N-acetylglucosamine transferase. In more specific embodiments, heparan sulfate modulators, and in specific embodiments heparan sulfate inhibitors, selectively modulate (e.g., promote or inhibit) one of xylosyltransfarase I, xylosyltransfarase II, galactosyltransferase I, galactosyltransferase II, glucuronosyltransferase I, glucuronosyltransferase II, N-acetylglucosamine transferase I, or N-acetylglucosamine transferase II. In still more specific embodiments, heparan sulfate inhibitors selectively inhibit xylosyltransfarase 1. In some specific embodiments, heparan sulfate inhibitors selectively inhibit xylosyltransfarase II. In some specific embodiments, heparan sulfate inhibitors selectively inhibit galactosyltransferase I. In some specific embodiments, heparan sulfate inhibitors selectively inhibit galactosyltransferase II. In some specific embodiments, heparan sulfate inhibitors selectively inhibit glucuronosyltransferase I. In some specific embodiments, heparan sulfate inhibitors selectively inhibit glucuronosyltransferase II. In some specific embodiments, heparan sulfate inhibitors selectively inhibit N-acetylglucosamine transferase I. In some specific embodiments, heparan sulfate inhibitors selectively inhibit N-acetylglucosamine transferase II.

In some embodiments, modulators, and in specific embodiments inhibitors, of heparan sulfate biosynthesis are utilized in any process described herein. In certain embodiments, modulators of heparan sulfate biosynthesis include promoters or inhibitors of heparan sulfate degradation. In certain embodiments, heparan sulfate inhibitors described herein are promoters of heparan sulfate degradation (e.g., activate or enhance the activity of enzymes that degrade heparan sulfate). In some embodiments, promoters of heparan sulfate degradation are useful or are used in a method treatment of a lysosomal storage disease, e.g., MPS, such as MPS I, MPS II, MPS IIIA, MPS IIIB, or any other MPS disease described herein (e.g., by administering a therapeutically effective dose of the modulator of heparan sulfate biosynthesis to an individual in need thereof). In certain embodiments, the promoter of heparan sulfate degradation is an agent that facilitates the degradation of heparan sulfate, including oligosaccharide fragments thereof (such as GlcNAc-GlcA-GlcNAc with 1-3 O-sulfation, GlcNAc-GlcA-GlcNS with 1-3 O sulfation, GlcNAc-IdoA-GlcNAc with 1-3 O sulfation, GlcNAc-IdoA-GlcNS with 1-sulfation, or the like). In specific embodiments, an promoter of heparan sulfate degradation is an agent that facilitates the degradation of heparan sulfate by modifying the heparan sulfate or by inhibiting the production of a specific type of heparan sulfate, including heparan sulfate oligosaccharide fragments, so as to produce a heparan sulfate, or fragment thereof, that is more easily degraded by an heparan sulfate degrading enzyme, such as β-glucuronidase (e.g., for the treatment of MPS VII), sulfamidase, such as N-sulfatase (e.g., for the treatment of MPS IIIA), N-acetyltransferase (e.g., for the treatment of MPS IIIC), glucosamidase, such as N-acetylglucoamidase (e.g., for the treatment of MPS IIIB), 2-O-sulfatase (e.g., for the treatment of MPS II), α-L-iduronidase (e.g., for the treatment of MPS I), 6-sulfatase (e.g., for the treatment of MPS IIID), or the like. In certain embodiments, heparan sulfate inhibitors described herein activate or promote the activity of β-glucuronidase (e.g., for the treatment of MPS VII). In some embodiments, heparan sulfate inhibitors described herein activate or promote the activity of sulfamidase, such as N-sulfatase (e.g., for the treatment of MPS IIIA). In certain embodiments, heparan sulfate inhibitors described herein activate or promote the activity of N-acetyltransferase (e.g., for the treatment of MPS IIIC). In some embodiments, heparan sulfate inhibitors described herein activate or promote the activity of glucosamidase, such as N-acetylglucoamidase (e.g., for the treatment of MPS IIIB). In certain embodiments, heparan sulfate inhibitors described herein activate or promote the activity of 2-O-sulfatase (e.g., for the treatment of MPS II). In some embodiments, heparan sulfate inhibitors described herein activate or promote the activity of α-L-iduronidase (e.g., for the treatment of MPS I). In certain embodiments, heparan sulfate inhibitors described herein activate or promote the activity of 6-sulfatase (e.g., for the treatment of MPS IIID). In certain instances, degradation of the heparan sulfate by the heparan sulfate degrading enzyme, e.g., one as discussed above, is facilitated by de-sulfating or partially de-sulfating the heparan sulfate (including fragments thereof). In some instances, degradation of the heparan sulfate by the heparan sulfate degrading enzyme, e.g., one as discussed above, is facilitated by inhibiting sulfation of the heparan sulfate (including fragments thereof). Thus, in specific embodiments, degradation of heparan sulfate by a heparan sulfate degrading enzyme is facilitated by contacting the heparan sulfate (including fragments thereof) with an effective amount of an inhibitor of heparan sulfate sulfation (e.g., an agent that inhibits an enzyme that sulfates the heparan sulfate, such as a sulfotransferase, or the like), e.g., an inhibitor of 2-O sulfation (e.g., compound 5), an inhibitor of 3-O sulfation, an inhibitor of 6-O sulfation, an inhibitor of N-sulfation, or the like. Likewise, in another specific embodiment, degradation of heparan sulfate by a heparan sulfate degrading enzyme is facilitated with an effective amount of agent that promotes heparan sulfate de-sulfation (e.g., an agent that itself de-sulfates the heparan sulfate or fragment thereof, an agent that activates an enzyme that de-sulfates the heparan sulfate or fragment thereof, or the like), e.g., a promoter of 2-O desulfation, a promoter of 3-O de-sulfation, a promoter of 6-O desulfation, a promoter of N-desulfation, or the like. In some embodiments, a heparan sulfate inhibitor provided herein is a promoter of 2-O desulfation. In certain embodiments, a heparan sulfate inhibitor provided herein is a promoter of 3-O de-sulfation. In some embodiments, a heparan sulfate inhibitor provided herein is a promoter of 6-O desulfation. In certain embodiments, a heparan sulfate inhibitor provided herein is a promoter of N-desulfation. In certain instances, provided herein is a method of treating a lysosomal storage disease (e.g., with substrate optimization therapy (SOT)) by administering a therapeutically effective amount of an agent that alters or modifies the nature of heparan sulfate produced and/or present in an individual in need thereof. In specific embodiments, the agent that alters or modifies the nature of heparan sulfate produced and/or present in an individual is an agent that facilitates the degradation of heparan sulfate, e.g., as described herein. In some specific embodiments, the lysosomal storage disease is characterized by an accumulation of heparan sulfate, or fragments thereof, comprising highly sulfated trisaccharide residues (e.g., GlcNAc-GlcA/IdoA-GlcNAc/S), such as disulfated trisaccharide residues, trisulfated trisaccharide residues, or tetrasulfated trisaccharide residues. In a specific embodiment, the lysosomal storage disease characterized by an accumulation of heparan sulfate, or fragments thereof, comprising highly sulfated trisaccharide residues is MPS IIIB.

In certain embodiments, heparan sulfate modulators, and in specific embodiments heparan sulfate inhibitors, described herein are small molecule organic compounds. Thus, in certain instances, heparan sulfate modulators, and in specific embodiments heparan sulfate inhibitors, utilized herein are not polypeptides or carbohydrates. In some embodiments, a small molecule organic compounds has a molecular weight of less than 2,000 g/mol, less than 1,500 g/mol, less than 1,000 g/mol, or less than 500 g/mol. In certain embodiments, a small molecule organic compounds has a molecular weight of less than 2,000 g/mol. In specific embodiments, a small molecule organic compounds has a molecular weight of less than 1,500 g/mol. In more specific embodiments, a small molecule organic compounds has a molecular weight of less than 1,000 g/mol. In still more specific embodiments, a small molecule organic compounds has a molecular weight of less than 500 g/mol.

In some embodiments, provided herein is a heparan sulfate biosynthesis modulator (e.g., a selective heparan sulfate biosynthesis inhibitor) having suitable cell availability and/or bioavailability to significantly effect the in cyto and/or in vivo biosynthesis of heparan sulfate when the heparan sulfate biosynthesis modulator is administered to a cell or individual, respectively. In certain instances, a significant effect is one wherein a measurable effect, a statistically significant effect, and/or a therapeutic effect is provided to the cell or individual. In certain specific embodiments, the specific heparan sulfate modulator (e.g., inhibitor of promoter) is substantially cell permeable (e.g., when in contact with a cell, a significant percentage/amount of the modulator permeates the cell membrane). In some embodiments, the heparan sulfate biosynthesis modulator provides a statistically significant effect and/or therapeutic effect in a cell or individual at a non-toxic concentration, a substantially non-toxic concentration, a concentration below LC₅₀, a concentration below LC₂₀, a concentration below LC₀₁, or the like.

### Compounds

In certain embodiments, heparan sulfate inhibitors as described herein have the structure of Formula III:

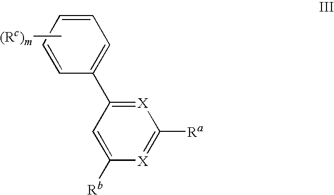

In some embodiments, R^{a} and R^{b} are, independently, substituted or unsubstituted cycloalkyl, substituted or unsubstituted aryl, substituted or unsubstituted cycloheteroalkyl, or substituted or unsubstituted heteroaryl. In certain embodiments, R^{c} each is independently alkyl, cycloalkyl, heteroalkyl, aryl, heteroaryl, heteroalicyclic hydroxy, alkoxy, aryloxy, alkylthio, arylthio, alkylsulfoxide, arylsulfoxide, ester, alkylsulfone, arylsulfone, cyano, halo, alkoyl, alkoyloxo, isocyanato, thiocyanato, isothiocyanato, nitro, haloalkyl, haloalkoxy, fluoroalkyl, amino, alkyl-amino, dialkyl-amino, or amido. In some embodiments, m is 0-5. In some embodiments, the heparan sulfate inhibitor is a pharmaceutically acceptable salt of a compound of Formula III.

In certain embodiments, R^{a} is substituted or unsubstituted cycloheteroalkyl. In some embodiments, R^{b} is substituted or unsubstituted aryl. In certain embodiments, each R^{c} is alkoxy. In some embodiments, m is 1-3. In more specific embodiments, m is 2.

In some embodiments, heparan sulfate inhibitors as described herein have the structure of Formula IV:

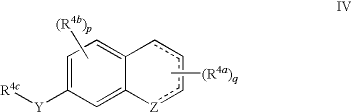

In certain embodiments, each R^{4a}, R^{4b}, and R^{4c} are, independently, substituted or unsubstituted alkyl, substituted or unsubstituted cycloalkyl, substituted or unsubstituted heteroalkyl, substituted or unsubstituted aryl, substituted or unsubstituted heteroaryl, substituted or unsubstituted heteroalicyclic substituted or unsubstituted hydroxy, substituted or unsubstituted alkoxy, substituted or unsubstituted aryloxy, substituted or unsubstituted alkylthio, substituted or unsubstituted arylthio, substituted or unsubstituted alkylsulfoxide, substituted or unsubstituted arylsulfoxide, substituted or unsubstituted ester, substituted or unsubstituted alkylsulfone, substituted or unsubstituted arylsulfone, cyano, halo, alkoyl, alkoyloxo, isocyanato, thiocyanato, isothiocyanato, nitro, haloalkyl, haloalkoxy, fluoroalkyl, substituted or unsubstituted amino, substituted or unsubstituted alkoyl amino, substituted or unsubstituted alkyl-amino, substituted or unsubstituted dialkyl-amino, or substituted or unsubstituted amido. In some embodiments, a pair of R^{4a} on a single carbon are taken together to form an oxo, a thioxo, or ═NR^{4d}. In some embodiments, Y is O, S, NR^{4d}, or C(R^{4d})₂. In certain embodiments, Z is O, S, N(R^{4d})ᵣ, C(R^{4d})ₛ, wherein r is 0-1 and s is 1-2. Each [symbol] bond is a single or double bond. Each R^{4d} is independently hydrogen, alkyl, cycloalkyl, heteroalkyl, aryl, heteroaryl, heteroalicyclic hydroxy, alkoxy, aryloxy, alkylthio, arylthio, alkylsulfoxide, arylsulfoxide, ester, alkylsulfone, arylsulfone, cyano, halo, alkoyl, alkoyloxo, isocyanato, thiocyanato, isothiocyanato, nitro, haloalkyl, haloalkoxy, fluoroalkyl, amino, alkyl-amino, dialkyl-amino, or amido. In some embodiments, p is 0-3 and q is 0-6. In some embodiments, the heparan sulfate inhibitor is a pharmaceutically acceptable salt of a compound of Formula IV.

In specific embodiments, R^{4c} is substituted or unsubstituted phenyl or substituted or unsubstituted thiophene. In some embodiments, Y is O or C(R^{4d})₂. In certain embodiments, Z is O or NR^{4d}. In a specific embodiment, the compound of Formula IV is 4-ethyl-7-(4-nitro-2-(trifluoromethyl)phenoxy)-2H-chromen-2-one.

In some embodiments, heparan sulfate inhibitors described herein have the structure of Formula V:

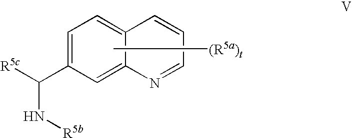

In certain embodiments, each R^{5a}, R^{5b}, and R^{5c} are, independently, substituted or unsubstituted alkyl, substituted or unsubstituted cycloalkyl, substituted or unsubstituted heteroalkyl, substituted or unsubstituted aryl, substituted or unsubstituted heteroaryl, substituted or unsubstituted heteroalicyclic substituted or unsubstituted hydroxy, substituted or unsubstituted alkoxy, substituted or unsubstituted aryloxy, substituted or unsubstituted alkylthio, substituted or unsubstituted arylthio, substituted or unsubstituted alkylsulfoxide, substituted or unsubstituted arylsulfoxide, substituted or unsubstituted ester, substituted or unsubstituted alkylsulfone, substituted or unsubstituted arylsulfone, cyano, halo, alkoyl, alkoyloxo, isocyanato, thiocyanato, isothiocyanato, nitro, haloalkyl, haloalkoxy, fluoroalkyl, amino, substituted or unsubstituted alkyl-amino, substituted or unsubstituted dialkyl-amino, or amido. In some embodiments, t is 0-6. In specific embodiments, R^{5c} is substituted or unsubstituted phenyl or substituted or unsubstituted thiophene. In specific embodiments, the compound is 7-((3-chlorophenyl)(pyridin-2-ylamino)methyl)-2-methylquinolin-8-ol, 7-((2-fluorophenyl)(pyridin-2-ylamino)methyl)-2-methylquinolin-8-ol, 7-((4-chlorophenyl)(acetylamino)methyl)-5-nitroquinolin-8-ol, or 7-((thiophen-2-yl)(isobutoylamino)methyl)-2-methylquinolin-8-ol. In some embodiments, the heparan sulfate inhibitor is a pharmaceutically acceptable salt of a compound of Formula V.

In some embodiments, heparan sulfate inhibitors described herein have the structure of Formula VI:

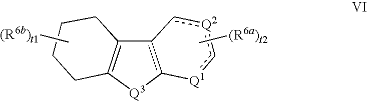

In certain embodiments, each R^{6a} and R^{6b} are, independently, substituted or unsubstituted alkyl, substituted or unsubstituted cycloalkyl, substituted or unsubstituted heteroalkyl, substituted or unsubstituted aryl, substituted or unsubstituted heteroaryl, substituted or unsubstituted heteroalicyclic substituted or unsubstituted hydroxy, substituted or unsubstituted alkoxy, substituted or unsubstituted aryloxy, substituted or unsubstituted alkylthio, substituted or unsubstituted arylthio, substituted or unsubstituted alkylsulfoxide, substituted or unsubstituted arylsulfoxide, substituted or unsubstituted ester, substituted or unsubstituted alkylsulfone, substituted or unsubstituted arylsulfone, cyano, oxo, substituted or unsubstituted imino, halo, alkoyl, alkoyloxo, isocyanato, thiocyanato, isothiocyanato, nitro, haloalkyl, haloalkoxy, fluoroalkyl, amino, substituted or unsubstituted alkyl-amino, substituted or unsubstituted dialkyl-amino, or amido. In various embodiments, t1 is 0-8 and t2 is 0-4. In some embodiments, each of Q1, Q2, and Q3 is independently O, S, N, NR^{6c}, CR^{6c} or C(R^{6c})₂. Each [symbol] bond is a single or double bond. Each R^{6c} is independently hydrogen, alkyl, cycloalkyl, heteroalkyl, aryl, heteroaryl, heteroalicyclic hydroxy, alkoxy, aryloxy, alkylthio, arylthio, alkylsulfoxide, arylsulfoxide, ester, alkylsulfone, arylsulfone, cyano, halo, alkoyl, alkoyloxo, isocyanato, thiocyanato, isothiocyanato, nitro, haloalkyl, haloalkoxy, fluoroalkyl, amino, alkyl-amino, dialkyl-amino, or amido. In specific embodiments, one R^{6a} is oxo, R^{6b} is tert-butyl, one R^{6a} is trifluoromethyl, t1 is 1, t2 is 2, Q1 is N, Q2 is NH, and/or Q3 is S. In some embodiments, the heparan sulfate inhibitor is a pharmaceutically acceptable salt of a compound of Formula VI.

In some embodiments, heparan sulfate inhibitors described herein have the structure of Formula VII:

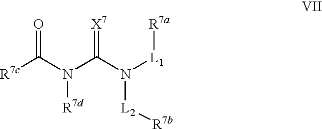

In certain embodiments, each R^{7a}, R^{7b}, and R^{7c} are, independently, substituted or unsubstituted cycloalkyl, substituted or unsubstituted aryl, substituted or unsubstituted heteroaryl, or substituted or unsubstituted heteroalicyclic. In various embodiments, X⁷ is O, S, NR^{7e} or C(R^{7e})₂. Each R^{7e} and R^{7d} is independently hydrogen, alkyl, cycloalkyl, heteroalkyl, aryl, heteroaryl, heteroalicyclic hydroxy, alkoxy, aryloxy, alkylthio, arylthio, alkylsulfoxide, arylsulfoxide, ester, alkylsulfone, arylsulfone, cyano, halo, alkoyl, alkoyloxo, isocyanato, thiocyanato, isothiocyanato, nitro, haloalkyl, haloalkoxy, fluoroalkyl, amino, alkyl-amino, dialkyl-amino, or amido. Each of L1 and L2 are independently selected from a bond or a —CR^{7e}₂)ₙ₇—, wherein n7 is 0-6. In a specific embodiment, a compound of Formula VII is 3-chloro-N-(dibenzylcarbamothioyl)benzamide. In some embodiments, the heparan sulfate inhibitor is a pharmaceutically acceptable salt of a compound of Formula VII.

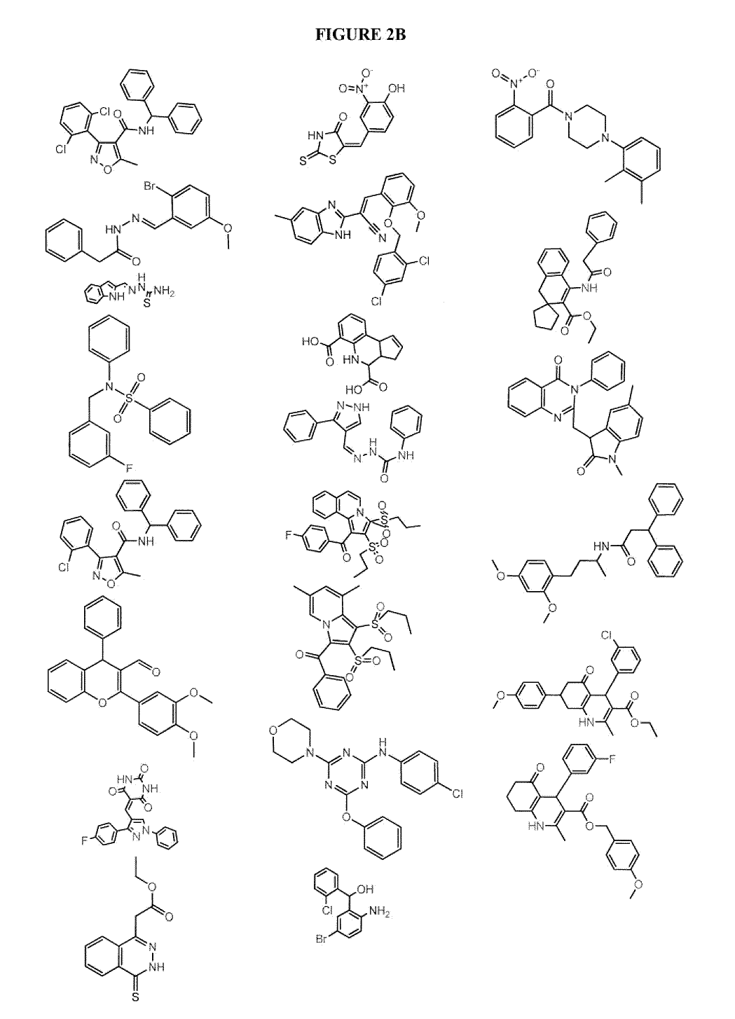

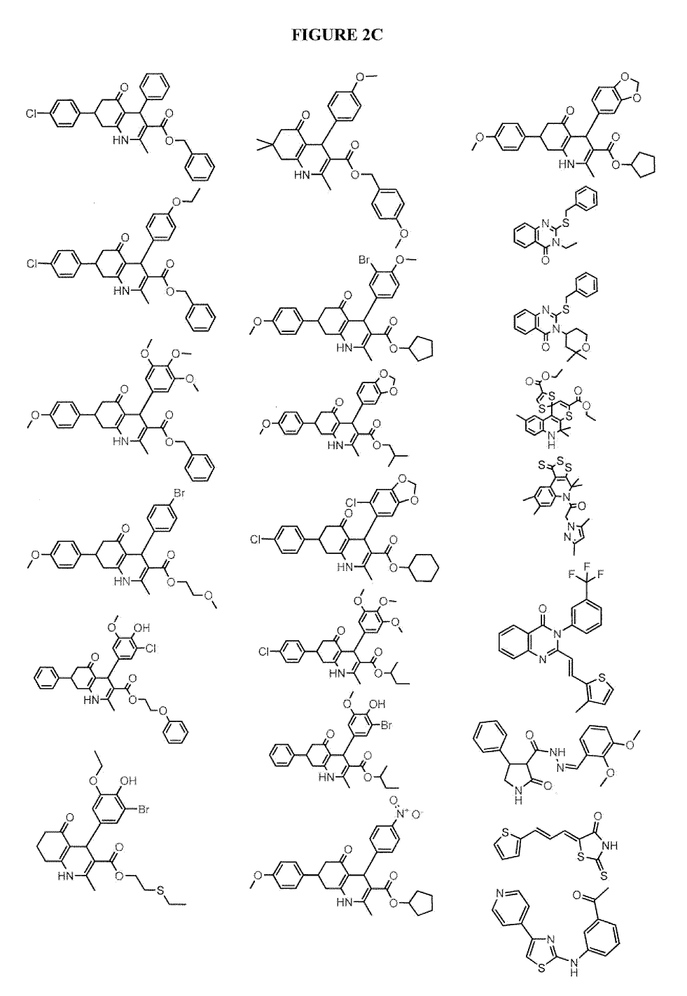

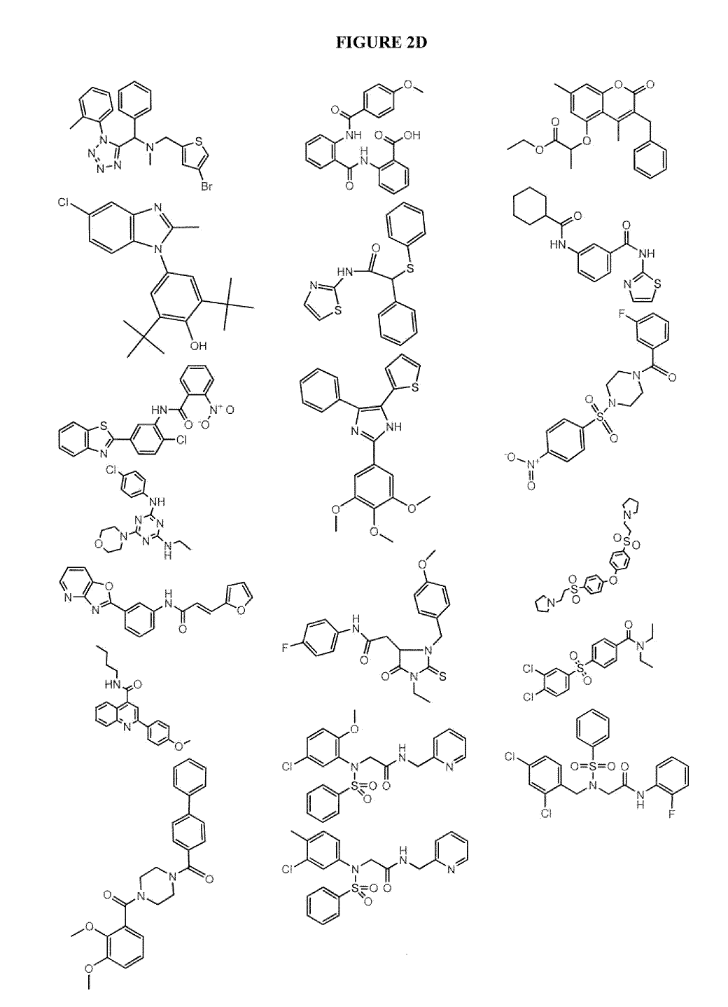

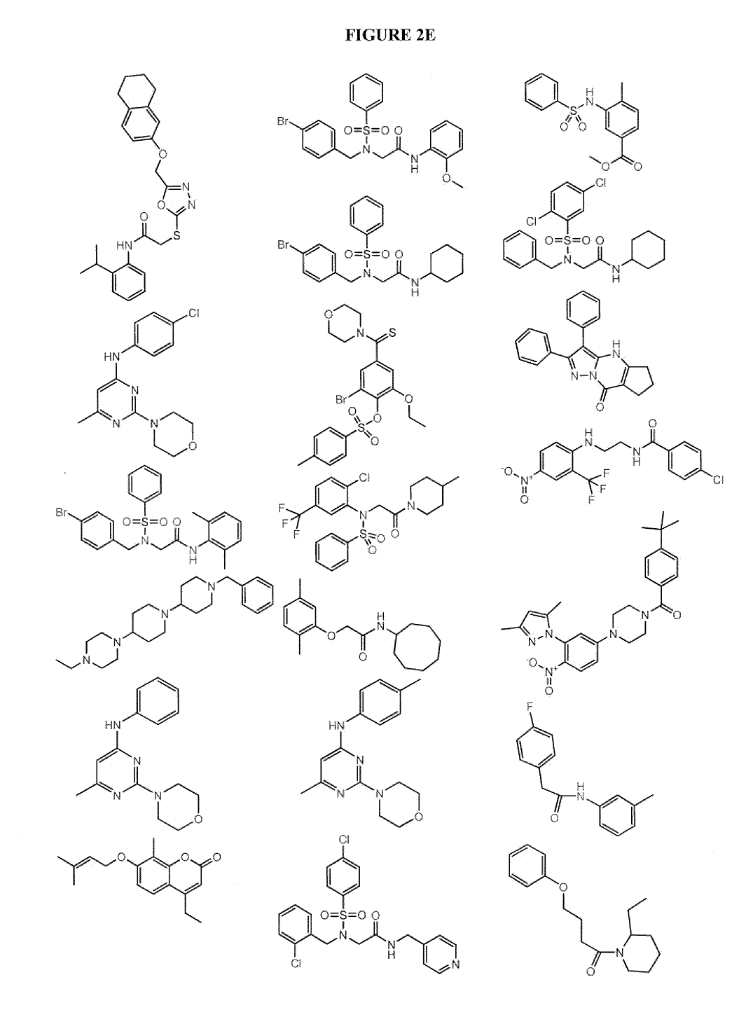

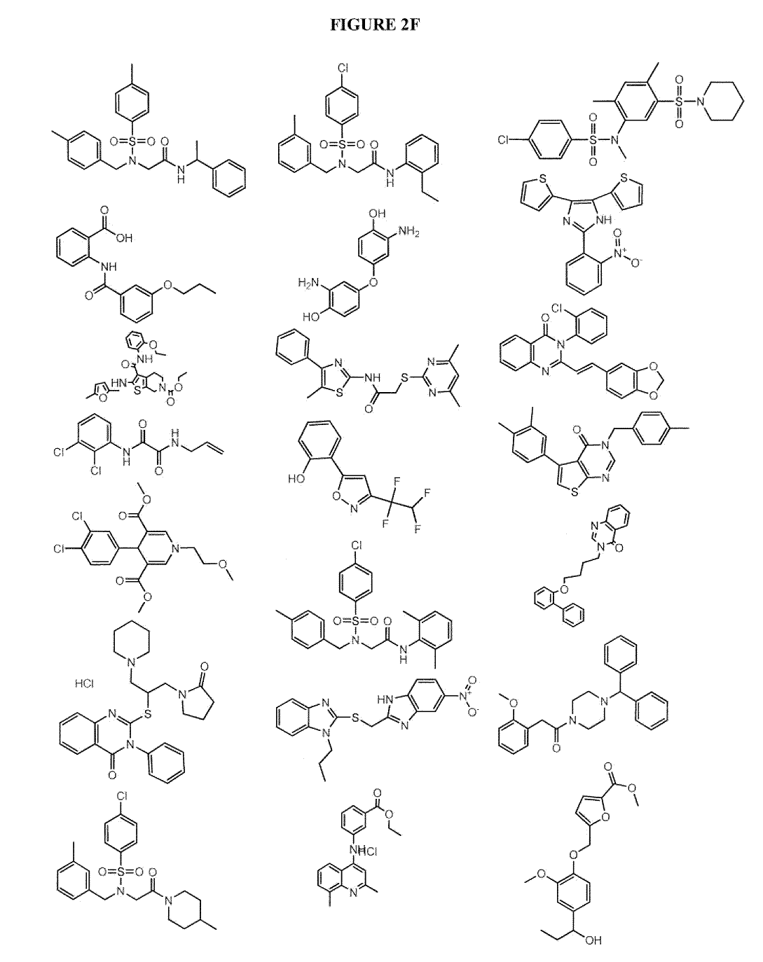

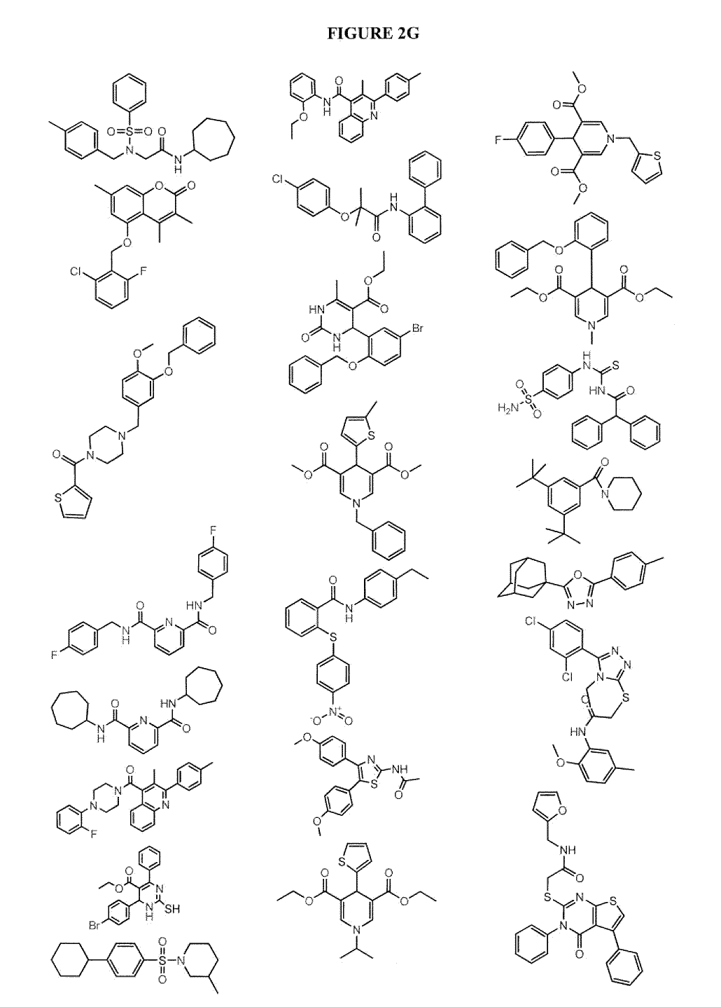

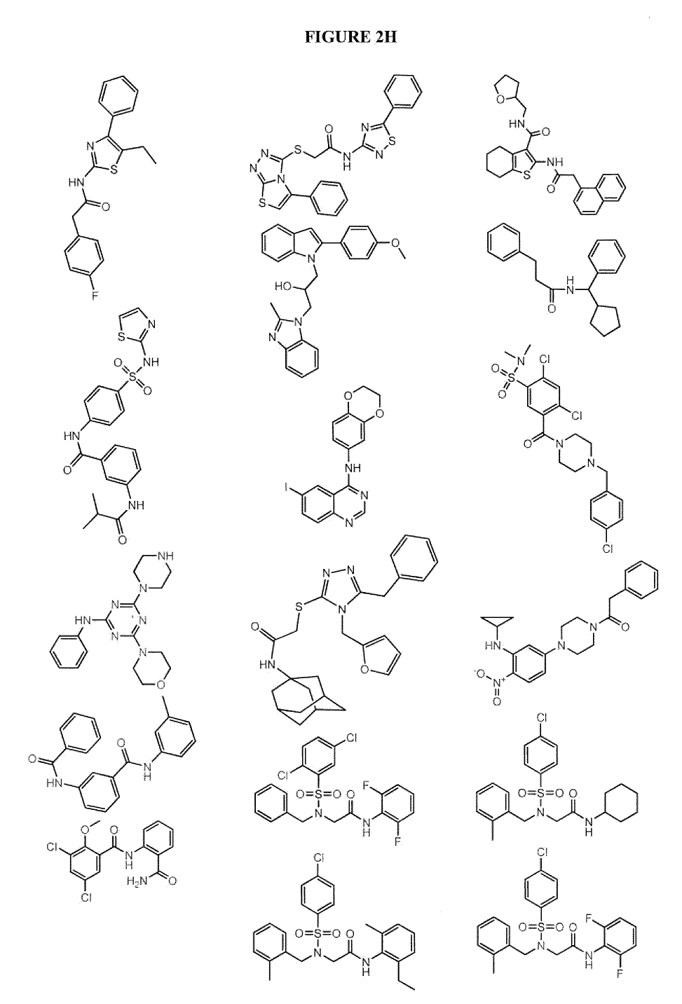

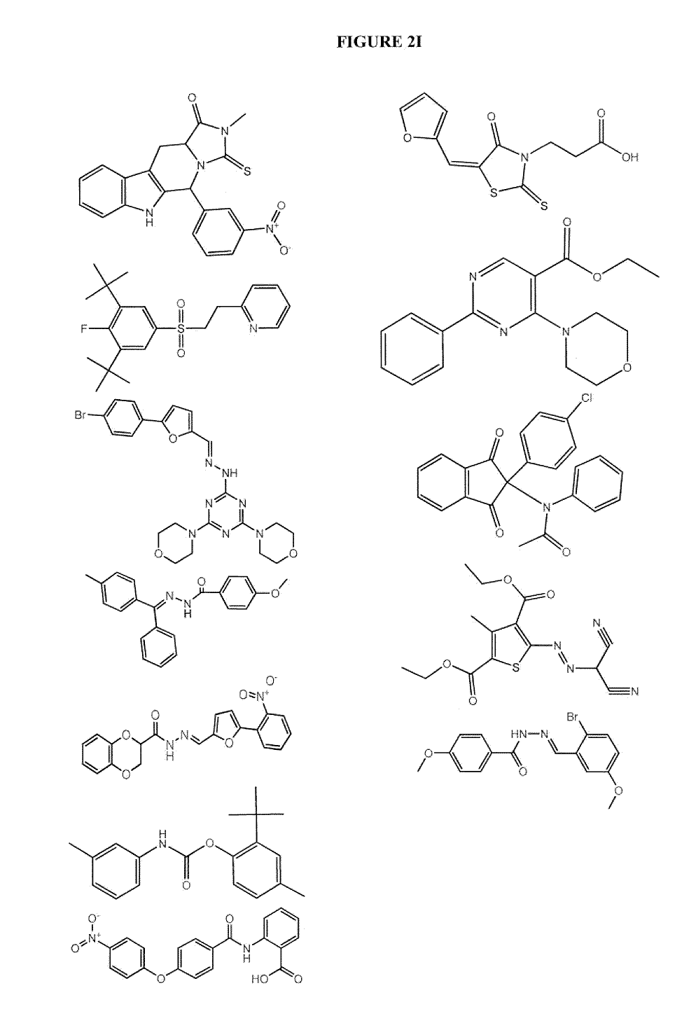

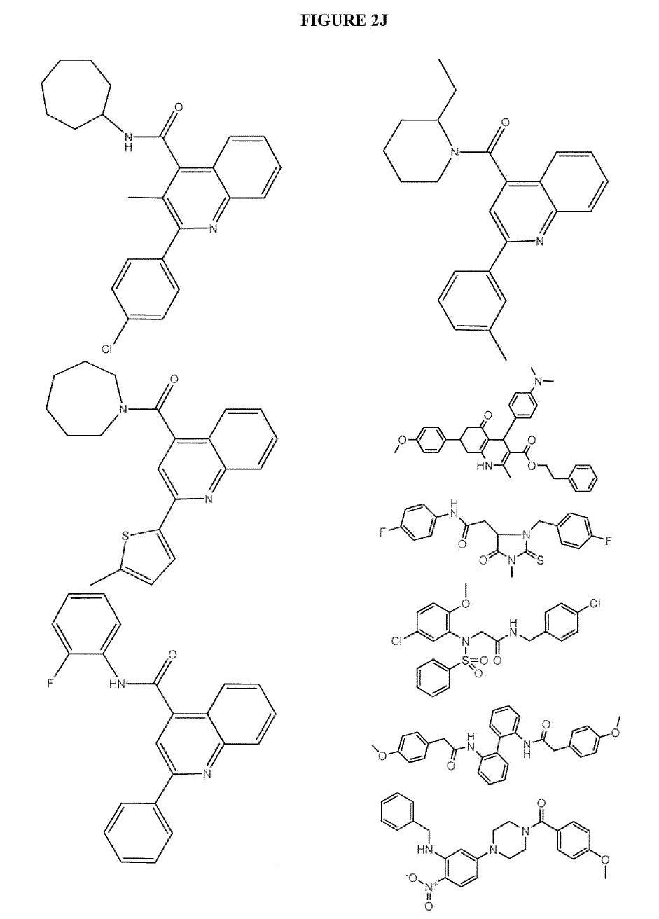

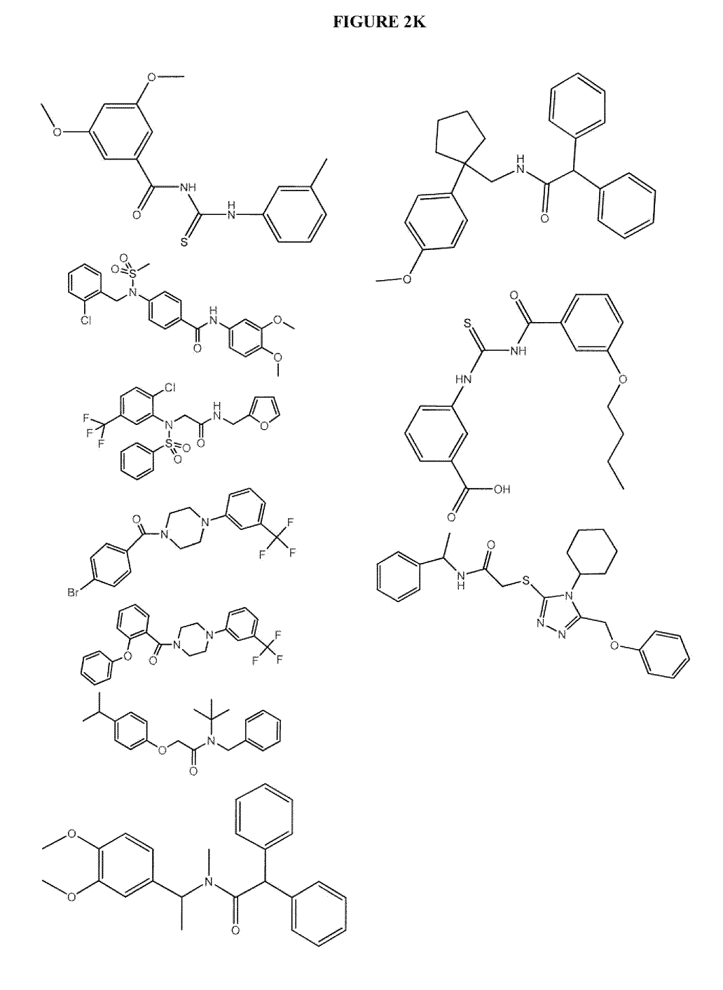

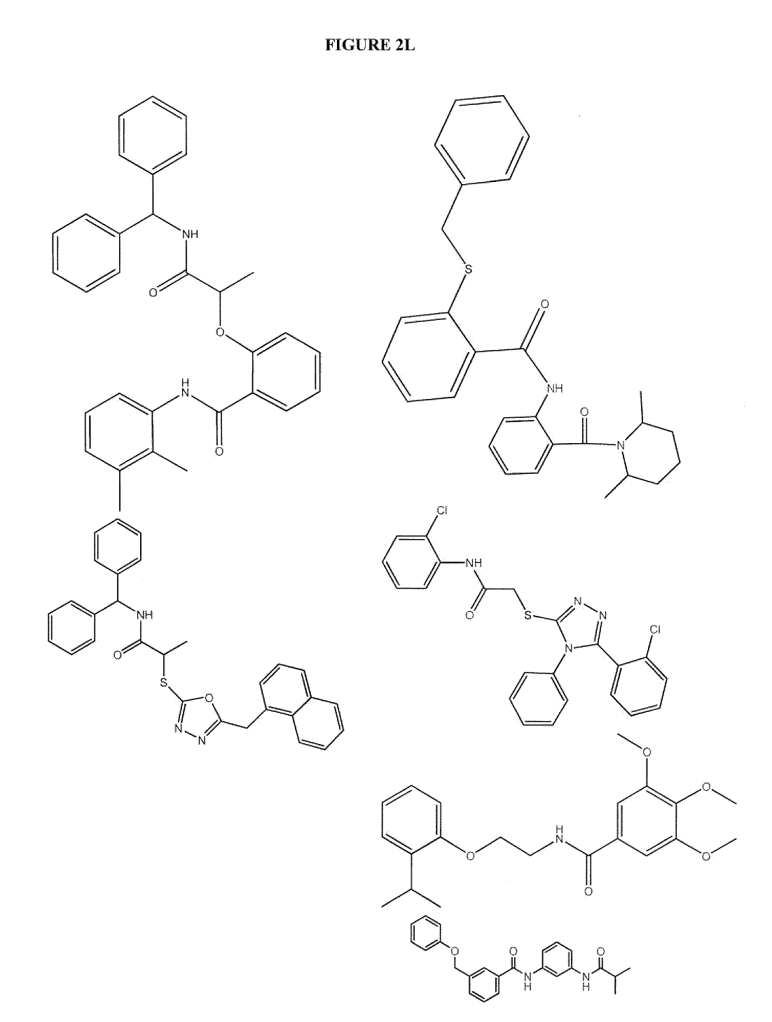

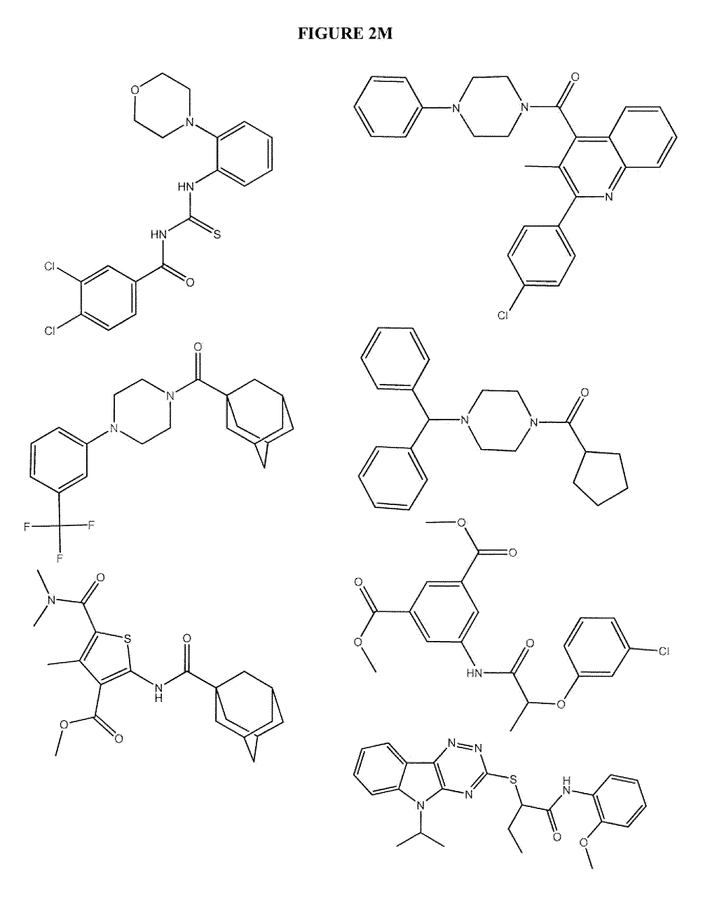

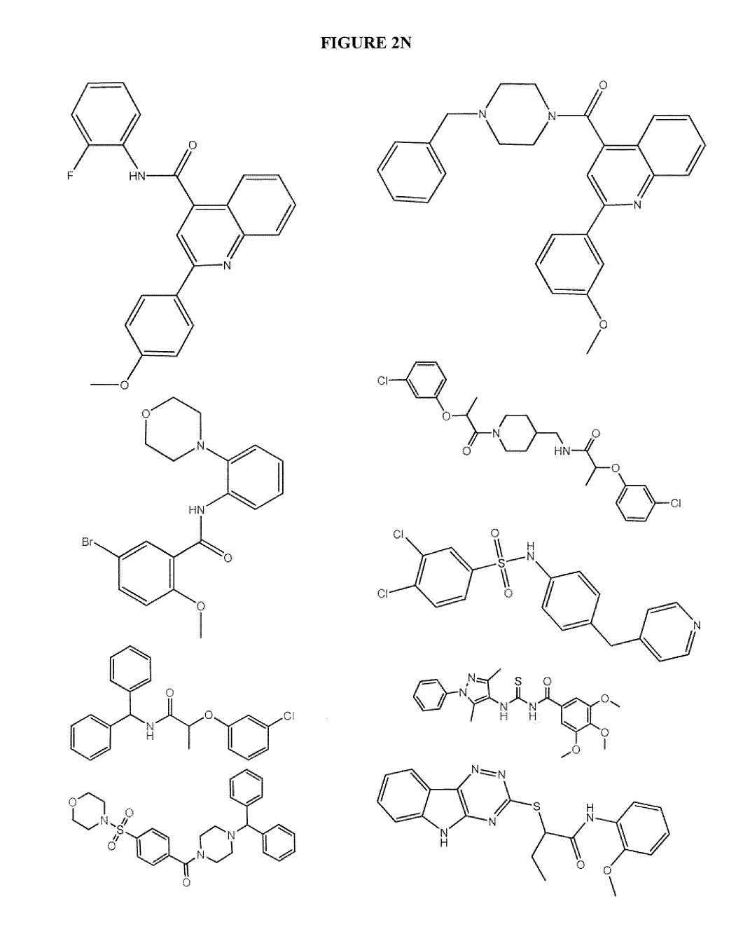

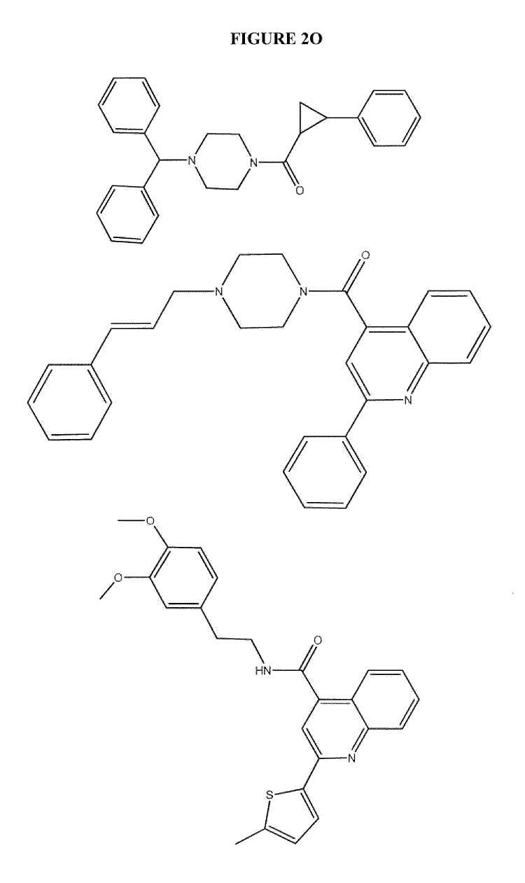

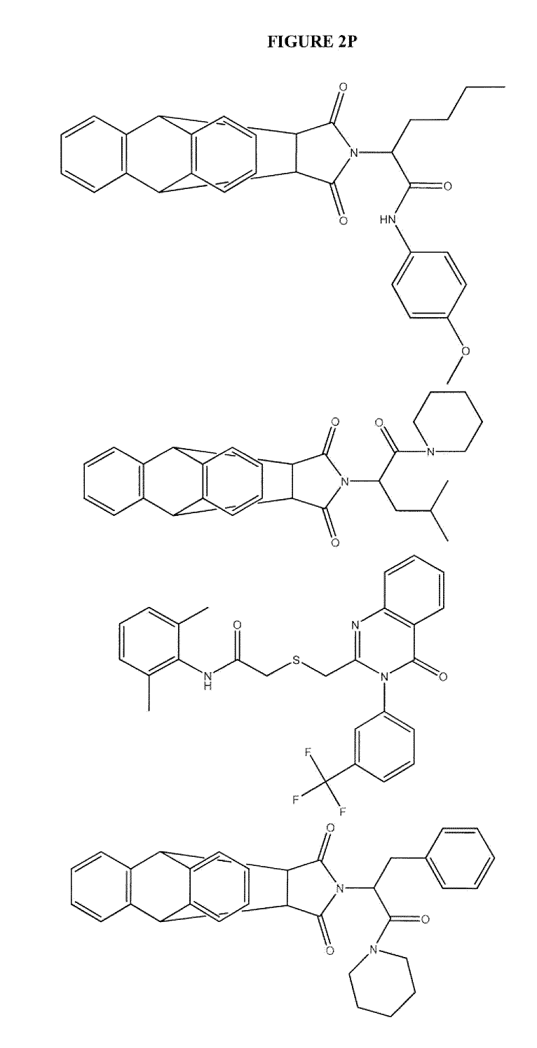

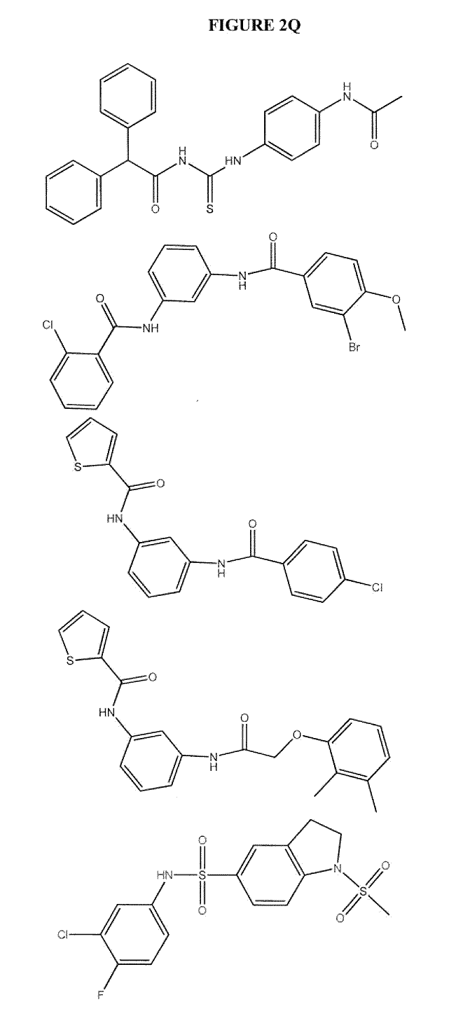

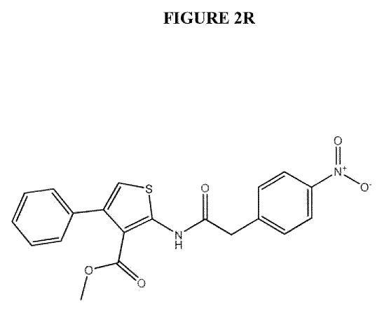

In certain embodiments, heparan sulfate inhibitors described herein have one of the structures of Table 1. Moreover, the compounds of FIG. 2 have been identified as selective inhibitors of heparan sulfate according to a screening process described herein. In particular, the compounds described in FIG. 2 selectively inhibit the heparan sulfate, binding lectin FGF, while not affecting the N-linked glycan-lectin binding, O-linked glycan-lectin binding, or ganglioside-lectin binding. Using similar methods, 326 inhibitors of heparan sulfate biosynthesis have been identified; 268 compounds have been identified as selectively inhibiting heparan sulfate (i.e., HS-lectin (FGF) binding), but not affecting N-linked glycans (i.e., N-linked glycan-lectin binding); 309 compounds have been identified as selectively inhibiting heparan sulfate (i.e., HS-lectin (FGF) binding), but not affecting O-linked glycans (i.e., O-linked glycan-lectin binding); and 281 compounds have been identified as selectively inhibiting heparan sulfate (i.e., HS-lectin (FGF) binding), but not affecting gangliosides (i.e., ganglioside-lectin binding).

TABLE 1

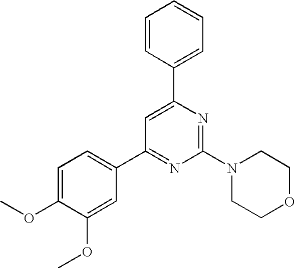

1

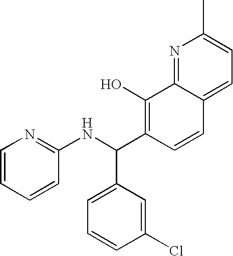

2

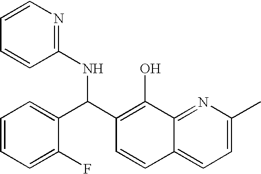

3

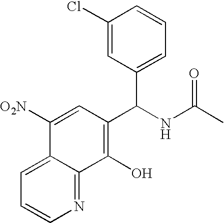

4

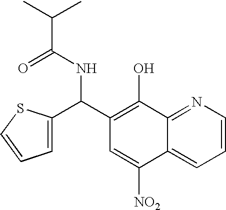

5

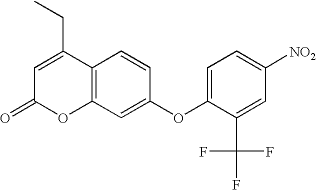

6

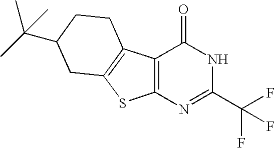

7

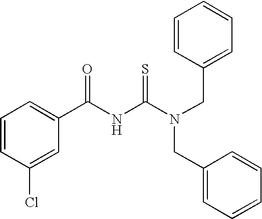

8

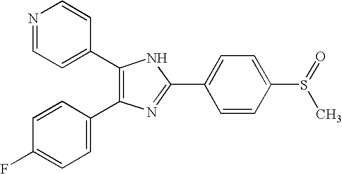

9

In certain embodiments, compounds described herein have one or more chiral centers. As such, all stereoisomers are envisioned herein. In various embodiments, compounds described herein are present in optically active or racemic forms. It is to be understood that the modulator compounds described herein encompasses racemic, optically-active, regioisomeric and stereoisomeric forms, or combinations thereof that possess the therapeutically useful properties described herein. Preparation of optically active forms is achieve in any suitable manner, including by way of non-limiting example, by resolution of the racemic form by recrystallization techniques, by synthesis from optically-active starting materials, by chiral synthesis, or by chromatographic separation using a chiral stationary phase. In some embodiments, mixtures of one or more isomer is utilized as the therapeutic compound described herein. In certain embodiments, compounds described herein contains one or more chiral centers. These compounds are prepared by any means, including entioselective synthesis and/or separation of a mixture of enantiomers and/or diastereomers. Resolution of therapeutic compounds and isomers thereof is achieved by any means including, by way of non-limiting example, chemical processes, enzymatic processes, fractional crystallization, distillation, chromatography, and the like.

The compounds described herein, and other related compounds having different substituents are synthesized using techniques and materials described herein and as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, ADVANCED ORGANIC CHEMISTRY 4^{th} Ed., (Wiley 1992); Carey and Sundberg, ADVANCED ORGANIC CHEMISTRY 4^{th} Ed., Vols. A and B (Plenum 2000, 2001), and Green and Wuts, PROTECTIVE GROUPS IN ORGANIC SYNTHESIS 3^{rd} Ed., (Wiley 1999) (all of which are incorporated by reference for such disclosures). General methods for the preparation of compound as disclosed herein are modified by the use of appropriate reagents and conditions, for the introduction of the various moieties found in the formulae as provided herein. As a guide the following synthetic methods are utilized.

Compounds described herein are synthesized starting from compounds that are available from commercial sources or that are prepared using procedures outlined herein.

Formation of Covalent Linkages by Reaction of an Electrophile with a Nucleophile

The compounds described herein are modified using various electrophiles and/or nucleophiles to form new functional groups or substituents. Table A entitled “Examples of Covalent Linkages and Precursors Thereof” lists selected non-limiting examples of covalent linkages and precursor functional groups which yield the covalent linkages. Table A is used as guidance toward the variety of electrophiles and nucleophiles combinations available that provide covalent linkages. Precursor functional groups are shown as electrophilic groups and nucleophilic groups.

TABLE A
Examples of Covalent Linkages and Precursors Thereof

| Covalent Linkage Product | Electrophile | Nucleophile |
|---|---|---|
| Carboxamides | Activated esters | amines/anilines |
| Carboxamides | acyl azides | amines/anilines |
| Carboxamides | acyl halides | amines/anilines |
| Esters | acyl halides | alcohols/phenols |
| Esters | acyl nitriles | alcohols/phenols |
| Carboxamides | acyl nitriles | amines/anilines |
| Imines | Aldehydes | amines/anilines |
| Hydrazones | aldehydes or ketones | Hydrazines |
| Oximes | aldehydes or ketones | Hydroxylamines |
| Alkyl amines | alkyl halides | amines/anilines |
| Esters | alkyl halides | carboxylic acids |
| Thioethers | alkyl halides | Thiols |
| Ethers | alkyl halides | alcohols/phenols |
| Thioethers | alkyl sulfonates | Thiols |
| Esters | alkyl sulfonates | carboxylic acids |
| Ethers | alkyl sulfonates | alcohols/phenols |
| Esters | Anhydrides | alcohols/phenols |
| Carboxamides | Anhydrides | amines/anilines |
| Thiophenols | aryl halides | Thiols |
| Aryl amines | aryl halides | Amines |
| Thioethers | Azindines | Thiols |
| Boronate esters | Boronates | Glycols |
| Carboxamides | carboxylic acids | amines/anilines |
| Esters | carboxylic acids | Alcohols |
| hydrazines | Hydrazides | carboxylic acids |
| N-acylureas or Anhydrides | carbodiimides | carboxylic acids |
| Esters | diazoalkanes | carboxylic acids |
| Thioethers | Epoxides | Thiols |
| Thioethers | haloacetamides | Thiols |
| Ammotriazines | halotriazines | amines/anilines |
| Triazinyl ethers | halotriazines | alcohols/phenols |
| Amidines | imido esters | amines/anilines |
| Ureas | Isocyanates | amines/anilines |
| Urethanes | Isocyanates | alcohols/phenols |
| Thioureas | isothiocyanates | amines/anilines |
| Thioethers | Maleimides | Thiols |
| Phosphite esters | phosphoramidites | Alcohols |
| Silyl ethers | silyl halides | Alcohols |
| Alkyl amines | sulfonate esters | amines/anilines |
| Thioethers | sulfonate esters | Thiols |
| Esters | sulfonate esters | carboxylic acids |
| Ethers | sulfonate esters | Alcohols |
| Sulfonamides | sulfonyl halides | amines/anilines |
| Sulfonate esters | sulfonyl halides | phenols/alcohols |

### Use of Protecting Groups

In the reactions described, it is necessary to protect reactive functional groups, for example hydroxy, amino, imino, thio or carboxy groups, where these are desired in the final product, in order to avoid their unwanted participation in reactions. Protecting groups are used to block some or all of the reactive moieties and prevent such groups from participating in chemical reactions until the protective group is removed. In some embodiments it is contemplated that each protective group be removable by a different means. Protective groups that are cleaved under totally disparate reaction conditions fulfill the requirement of differential removal.

In some embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl in the presence of amines blocked with acid labile groups such as t-butyl carbamate or with carbamates that are both acid and base stable but hydrolytically removable.

In some embodiments carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in then presence of acid- and base-protecting groups since the former are stable and are subsequently removed by metal or pi-acid catalysts. For example, an allyl-blocked carboxylic acid is deprotected with a Pd⁰-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups are selected from:

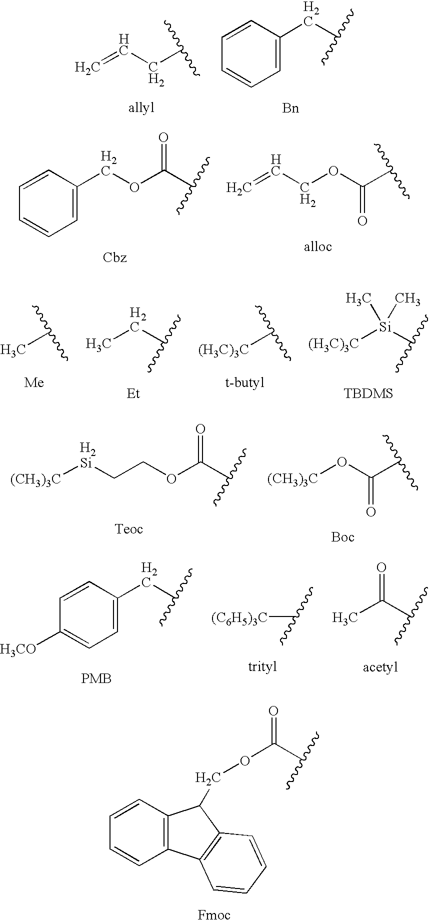

Other protecting groups, plus a detailed description of techniques applicable to the creation of protecting groups and their removal are described in Greene and Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosures.

### GENERAL DEFINITIONS

The term “subject”, “patient” or “individual” are used interchangeably herein and refer to mammals and non-mammals, e.g., suffering from a disorder described herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The terms “treat,” “treating” or “treatment,” and other grammatical equivalents as used herein, include alleviating, inhibiting or reducing symptoms, reducing or inhibiting severity of, reducing incidence of, prophylactic treatment of, reducing or inhibiting recurrence of, delaying onset of, delaying recurrence of, abating or ameliorating a disease or condition symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms further include achieving a therapeutic benefit. By therapeutic benefit is meant eradication or amelioration of the underlying disorder being treated, and/or the eradication or amelioration of one or more of the physiological symptoms associated with the underlying disorder such that an improvement is observed in the patient.

The terms “prevent,” “preventing” or “prevention,” and other grammatical equivalents as used herein, include preventing additional symptoms, preventing the underlying metabolic causes of symptoms, inhibiting the disease or condition, e.g., arresting the development of the disease or condition and are intended to include prophylaxis. The terms further include achieving a prophylactic benefit. For prophylactic benefit, the compositions are optionally administered to a patient at risk of developing a particular disease, to a patient reporting one or more of the physiological symptoms of a disease, or to a patient at risk of reoccurrence of the disease.

Where combination treatments or prevention methods are contemplated, it is not intended that the agents described herein be limited by the particular nature of the combination. For example, the agents described herein are optionally administered in combination as simple mixtures as well as chemical hybrids. An example of the latter is where the agent is covalently linked to a targeting carrier or to an active pharmaceutical. Covalent binding can be accomplished in many ways, such as, though not limited to, the use of a commercially available cross-linking agent. Furthermore, combination treatments are optionally administered separately or concomitantly.

As used herein, the terms “pharmaceutical combination”, “administering an additional therapy”, “administering an additional therapeutic agent” and the like refer to a pharmaceutical therapy resulting from the mixing or combining of more than one active ingredient and includes both fixed and non-fixed combinations of the active ingredients. The term “fixed combination” means that at least one of the agents described herein, and at least one co-agent, are both administered to a patient simultaneously in the form of a single entity or dosage. The term “non-fixed combination” means that at least one of the agents described herein, and at least one co-agent, are administered to a patient as separate entities either simultaneously, concurrently or sequentially with variable intervening time limits, wherein such administration provides effective levels of the two or more agents in the body of the patient. In some instances, the co-agent is administered once or for a period of time, after which the agent is administered once or over a period of time. In other instances, the co-agent is administered for a period of time, after which, a therapy involving the administration of both the co-agent and the agent are administered. In still other embodiments, the agent is administered once or over a period of time, after which, the co-agent is administered once or over a period of time. These also apply to cocktail therapies, e.g. the administration of three or more active ingredients.

As used herein, the terms “co-administration”, “administered in combination with” and their grammatical equivalents are meant to encompass administration of the selected therapeutic agents to a single patient, and are intended to include treatment regimens in which the agents are administered by the same or different route of administration or at the same or different times. In some embodiments the agents described herein will be co-administered with other agents. These terms encompass administration of two or more agents to an animal so that both agents and/or their metabolites are present in the animal at the same time. They include simultaneous administration in separate compositions, administration at different times in separate compositions, and/or administration in a composition in which both agents are present. Thus, in some embodiments, the agents described herein and the other agent(s) are administered in a single composition. In some embodiments, the agents described herein and the other agent(s) are admixed in the composition.

The terms “effective amount” or “therapeutically effective amount” as used herein, refer to a sufficient amount of at least one agent being administered which achieve a desired result, e.g., to relieve to some extent one or more symptoms of a disease or condition being treated. In certain instances, the result is a reduction and/or alleviation of the signs, symptoms, or causes of a disease, or any other desired alteration of a biological system. In specific instances, the result is the alteration of or the disruption of the structure of endogenous heparan sulfate such that the binding ability, signaling ability or combination thereof of the heparan sulfate is inhibited or reduced. In certain instances, an “effective amount” for therapeutic uses is the amount of the composition comprising an agent as set forth herein required to provide a clinically significant decrease in a disease. An appropriate “effective” amount in any individual case is determined using any suitable technique, such as a dose escalation study.

The terms “administer,” “administering”, “administration,” and the like, as used herein, refer to the methods that may be used to enable delivery of agents or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the agents and methods described herein, e.g., as discussed in Goodman and Gilman, The Pharmacological Basis of Therapeutics, current ed.; Pergamon; and Remington's, Pharmaceutical Sciences (current edition), Mack Publishing Co., Easton, Pa. In certain embodiments, the agents and compositions described herein are administered orally.

The term “pharmaceutically acceptable” as used herein, refers to a material that does not abrogate the biological activity or properties of the agents described herein, and is relatively nontoxic (i.e., the toxicity of the material significantly outweighs the benefit of the material). In some instances, a pharmaceutically acceptable material may be administered to an individual without causing significant undesirable biological effects or significantly interacting in a deleterious manner with any of the components of the composition in which it is contained.

The term “carrier” as used herein, refers to relatively nontoxic chemical agents that, in certain instances, facilitate the incorporation of an agent into cells or tissues.

“Pharmaceutically acceptable prodrug” as used herein, refers to any pharmaceutically acceptable salt, ester, salt of an ester or other derivative of an agent, which, upon administration to a recipient, is capable of providing, either directly or indirectly, a heparan sulfate modulator agent described herein or a pharmaceutically active metabolite or residue thereof. Particularly favored prodrugs are those that increase the bioavailability of the heparan sulfate modulator agents described herein when such agents are administered to a patient (e.g., by allowing an orally administered agent to be more readily absorbed into blood) or which enhance delivery of the parent agent to a biological compartment (e.g., the brain or lymphatic system). In various embodiments, pharmaceutically acceptable salts described herein include, by way of non-limiting example, a nitrate, chloride, bromide, phosphate, sulfate, acetate, hexafluorophosphate, citrate, gluconate, benzoate, propionate, butyrate, sulfosalicylate, maleate, laurate, malate, fumarate, succinate, tartrate, amsonate, pamoate, p-toluenesulfonate, mesylate and the like. Furthermore, pharmaceutically acceptable salts include, by way of non-limiting example, alkaline earth metal salts (e.g., calcium or magnesium), alkali metal salts (e.g., sodium or potassium), ammonium salts and the like.

The term “optionally substituted” or “substituted” means that the referenced group substituted with one or more additional group(s). In certain embodiments, the one or more additional group(s) are individually and independently selected from alkyl, cycloalkyl, heteroalkyl, aryl, heteroaryl, heteroalicyclic, hydroxy, alkoxy, aryloxy, alkylthio, arylthio, alkylsulfoxide, arylsulfoxide, ester, alkylsulfone, arylsulfone, cyano, halo, alkoyl, alkoyloxo, isocyanato, thiocyanato, isothiocyanato, nitro, haloalkyl, haloalkoxy, fluoroalkyl, amino, alkyl-amino, dialkyl-amino, amido.

An “alkyl” group refers to an aliphatic hydrocarbon group. Reference to an alkyl group includes “saturated alkyl” and/or “unsaturated alkyl”. The alkyl group, whether saturated or unsaturated, includes branched, straight chain, or cyclic groups. By way of example only, alkyl includes methyl, ethyl, propyl, iso-propyl, n-butyl, iso-butyl, sec-butyl, t-butyl, pentyl, iso-pentyl, neo-pentyl, and hexyl. In some embodiments, alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like. A “heteroalkyl” group substitutes any one of the carbons of the alkyl group with a heteroatom having the appropriate number of hydrogen atoms attached (e.g., a CH₂ group to an NH group or an O group).

An “alkoxy” group refers to a (alkyl)O— group, where alkyl is as defined herein.

The term “alkylamine” refers to the —N(alkyl)ₓH_{y} group, wherein alkyl is as defined herein and x and y are selected from the group x=1, y=1 and x=2, y=0. When x=2, the alkyl groups, taken together with the nitrogen to which they are attached, optionally form a cyclic ring system.

An “amide” is a chemical moiety with formula —C(O)NHR or —NHC(O)R, where R is selected from alkyl, cycloalkyl, aryl, heteroaryl (bonded through a ring carbon) and heteroalicyclic (bonded through a ring carbon).

The term “ester” refers to a chemical moiety with formula —C(═O)OR, where R is selected from the group consisting of alkyl, cycloalkyl, aryl, heteroaryl and heteroalicyclic.

The term “carbocyclic” or “carbocycle” refers to a ring wherein each of the atoms forming the ring is a carbon atom. Carbocycles includes aryl and cycloalkyl groups. The term thus distinguishes carbocycle from heterocycle (“heterocyclic”) in which the ring backbone contains at least one atom which is different from carbon (i.e a heteroatom). Heterocycle includes heteroaryl and heterocycloalkyl. Carbocycles and heterocycles disclosed herein are optionally substituted.

As used herein, the term “aryl” refers to an aromatic ring wherein each of the atoms forming the ring is a carbon atom. Aryl rings disclosed herein include rings having five, six, seven, eight, nine, or more than nine carbon atoms. Aryl groups are optionally substituted. Examples of aryl groups include, but are not limited to phenyl, and naphthalenyl.

The term “cycloalkyl” refers to a monocyclic or polycyclic non-aromatic radical, wherein each of the atoms forming the ring (i.e. skeletal atoms) is a carbon atom. In various embodiments, cycloalkyls are saturated, or partially unsaturated. In some embodiments, cycloalkyls are fused with an aromatic ring. Cycloalkyl groups include groups having from 3 to 10 ring atoms. Illustrative examples of cycloalkyl groups include, but are not limited to, the following moieties:

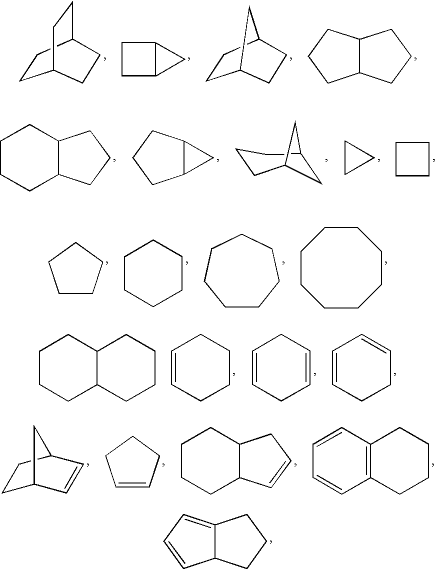

and the like. Monocyclic cycloalkyls include, but are not limited to, cyclopropyl, cyclobutyl, cyclopentyl, cyclohexyl, cycloheptyl, and cyclooctyl.

The term “heterocycle” refers to heteroaromatic and heteroalicyclic groups containing one to four ring heteroatoms each selected from O, S and N. In certain instances, each heterocyclic group has from 4 to 10 atoms in its ring system, and with the proviso that the ring of said group does not contain two adjacent O or S atoms. Non-aromatic heterocyclic groups include groups having 3 atoms in their ring system, but aromatic heterocyclic groups must have at least 5 atoms in their ring system. The heterocyclic groups include benzo-fused ring systems. An example of a 3-membered heterocyclic group is aziridinyl (derived from aziridine). An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

The terms “heteroaryl” or, alternatively, “heteroaromatic” refers to an aryl group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom. In certain embodiments, heteroaryl groups are monocyclic or polycyclic. Illustrative examples of heteroaryl groups include the following moieties:

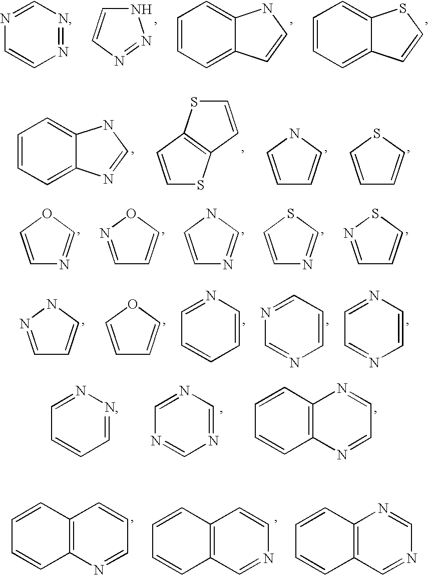

and the like.

A “heteroalicyclic” group or “heterocycloalkyl” group refers to a cycloalkyl group, wherein at least one skeletal ring atom is a heteroatom selected from nitrogen, oxygen and sulfur. In various embodiments, the radicals are with an aryl or heteroaryl. Illustrative examples of heterocycloalkyl groups, also referred to as non-aromatic heterocycles, include:

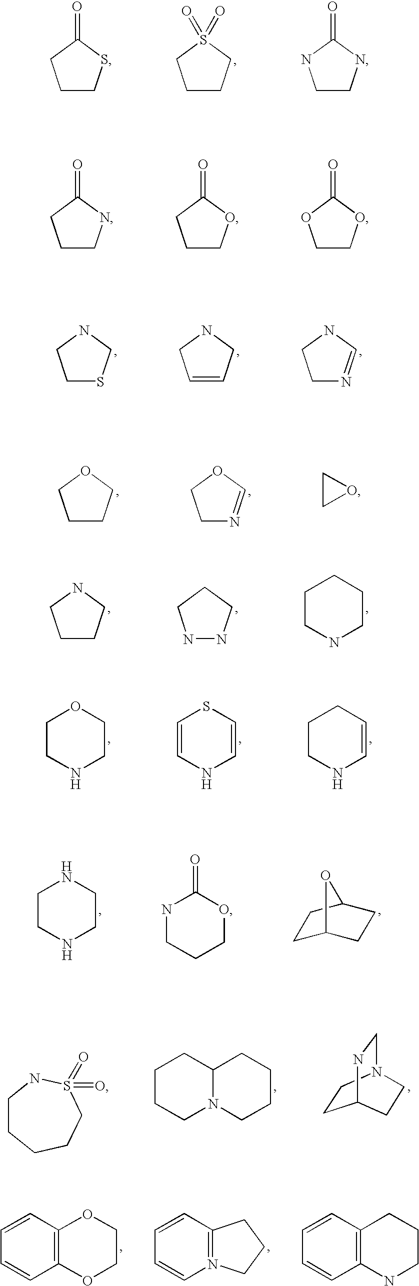

and the like. The term heteroalicyclic also includes all ring forms of the carbohydrates, including but not limited to the monosaccharides, the disaccharides and the oligosaccharides.

The term “halo” or, alternatively, “halogen” means fluoro, chloro, bromo and iodo.

The terms “haloalkyl,” and “haloalkoxy” include alkyl and alkoxy structures that are substituted with one or more halogens. In embodiments, where more than one halogen is included in the group, the halogens are the same or they are different. The terms “fluoroalkyl” and “fluoroalkoxy” include haloalkyl and haloalkoxy groups, respectively, in which the halo is fluorine.

The term “heteroalkyl” include optionally substituted alkyl, alkenyl and alkynyl radicals which have one or more skeletal chain atoms selected from an atom other than carbon, e.g., oxygen, nitrogen, sulfur, phosphorus, silicon, or combinations thereof. In certain embodiments, the heteroatom(s) is placed at any interior position of the heteroalkyl group. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. In some embodiments, up to two heteroatoms are consecutive, such as, by way of example, —CH₂—NH—OCH₃ and —CH₂—O—Si(CH₃)₃.

A “cyano” group refers to a —CN group.

An “isocyanato” group refers to a —NCO group.

A “thiocyanato” group refers to a —CNS group.

An “isothiocyanato” group refers to a —NCS group.

“Alkoyloxy” refers to a RC(═O)O— group, where R is selected from alkyl, cycloalkyl, heteroalkyl (bonded through a carbon), or cycloheteroalkyl (bonded through a carbon).

“Alkoyl” refers to a RC(═O)— group, where R is selected from alkyl, cycloalkyl, heteroalkyl (bonded through a carbon), or cycloheteroalkyl (bonded through a carbon).

### Methods

Provided in certain embodiments herein is a process for modifying the structure of heparan sulfate on a core protein (a heparan sulfate proteoglycan), comprising contacting a cell that translationally produces at least one core protein having at least one attached heparan sulfate moiety with an effective amount of any heparan sulfate modulator, and in specific embodiments heparan sulfate inhibitor, described herein. In some embodiments, the heparan sulfate modulator, and in specific embodiments heparan sulfate inhibitor, is a selective heparan sulfate inhibitor (as compared to the inhibition of the function, including lectin binding, of other GAGs and/or extracellular glycans), e.g., as described herein. In some embodiments, the selective heparan sulfate modulator, and in specific embodiments heparan sulfate inhibitor, is a modulator of (e.g., promotes one or more of, or inhibits one or more of) heparan sulfate glycosylation (e.g., modulates a heparan sulfate glycosyltransferase), heparan sulfate sulfation (e.g., modulates a heparan sulfate sulfotransferase), heparan sulfate epimerization (e.g., modulates a heparan sulfate epimerase), heparan sulfate phosphorylation (e.g., modulates a heparan sulfate kinase) or a combination thereof. In some specific embodiments, the heparan sulfate inhibitor inhibits heparan sulfate glycosylation. In certain specific embodiments, the heparan sulfate inhibitor inhibits heparan sulfate sulfation. In some specific embodiments, the heparan sulfate inhibitor inhibits heparan sulfate epimerization. In certain specific embodiments, the heparan sulfate inhibitor inhibits heparan sulfate phosphorylation.

In some embodiments, the heparan sulfate modulator, and in specific embodiments heparan sulfate inhibitor, modulates (e.g., promotes or inhibits) glycosyltransferase. In some embodiments, the modulator, and in specific embodiments inhibitor, of a heparan sulfate glycosyltransferase inhibits the synthesis of the linkage region, the initiation of heparan sulfate synthesis, the synthesis of heparan sulfate, or a combination thereof. In some embodiments, heparan sulfate modulators, and in specific embodiments heparan sulfate inhibitors, modulate (e.g., promote or inhibit) one or more of a heparan sulfate xylosyltransfarase, a heparan sulfate galactosyltransferase, a heparan sulfate glucuronosyltransferase, a heparan sulfate N-acetylglucosamine transferase, or combinations thereof. In more specific embodiments, heparan sulfate modulators, and in specific embodiments heparan sulfate inhibitors, selectively modulate (e.g., promote or inhibit) one or more of xylosyltransfarase I, xylosyltransfarase II, galactosyltransferase I, galactosyltransferase II, glucuronosyltransferase I, glucuronosyltransferase II, N-acetylglucosamine transferase I, N-acetylglucosamine transferase II, or a combination thereof. In some specific embodiments, the heparan sulfate inhibitor inhibits xylosyltransfarase I. In some specific embodiments, the heparan sulfate inhibitor inhibits xylosyltransfarase II. In some specific embodiments, the heparan sulfate inhibitor inhibits galactosyltransferase I. In some specific embodiments, the heparan sulfate inhibitor inhibits galactosyltransferase II. In some specific embodiments, the heparan sulfate inhibitor inhibits glucuronosyltransferase I. In some specific embodiments, the heparan sulfate inhibitor inhibits glucuronosyltransferase II. In some specific embodiments, the heparan sulfate inhibitor inhibits N-acetylglucosamine transferase I. In some specific embodiments, the heparan sulfate inhibitor inhibits N-acetylglucosamine transferase II.

In certain embodiments, heparan sulfate modulators, and in specific embodiments heparan sulfate inhibitors, that modulate sulfation modulate one or more sulfotransferase. In specific embodiments, the sulfotransferase is, by way of non-limiting example, a modulator (e.g., inhibitor or promoter) of one or more of a heparan sulfate O-sulfotransferase, a heparan sulfate N-sulfotransferase, or a combination thereof. In more specific embodiments, the heparan sulfate modulator, and in specific embodiments heparan sulfate inhibitor, modulates (e.g., inhibits or promotes) a heparan sulfate O-sulfotransferase such as, by way of non-limiting example, one or more of a 6-O sulfotransferase (of a glucosylamine group), a 3-O sulfotransferase (of a glucosylamine group), a 2-O sulfotransferase (of a uronic acid moiety, e.g., glucuronic acid or iduronic acid), or a combination thereof. In some specific embodiments, the heparan sulfate inhibitor inhibits 6-O sulfotransferase (of a glucosylamine group). In some specific embodiments, the heparan sulfate inhibitor inhibits 3-O sulfotransferase (of a glucosylamine group). In some specific embodiments, the heparan sulfate inhibitor inhibits a 2-O sulfotransferase (of a uronic acid moiety, e.g., glucuronic acid or iduronic acid).

In certain embodiments, the effective amount of the heparan sulfate modulator, and in specific embodiments heparan sulfate inhibitor, alters or disrupts the nature (e.g., alters or disrupts the acetylation, sulfation, O-sulfation, the 2-O sulfation, the 3-O sulfation, the 6-O sulfation, the N-sulfation, concentration of heparan sulfate, epimerization of heparan sulfate, chain length of heparan sulfate, or a combination thereof) of heparan sulfate compared to endogenous heparan sulfate in an amount sufficient to alter or disrupt heparan sulfate binding, heparan sulfate signaling, or a combination thereof. In specific embodiments, the heparan sulfate inhibitor described herein alters or disrupts the nature of the heparan sulfate such that it inhibits heparan sulfate signaling. In other specific embodiments, the heparan sulfate inhibitor described herein alters or disrupts the nature of the heparan sulfate such that it inhibits heparan sulfate binding (e.g., to FGF). In more specific embodiments, the heparan sulfate inhibitor described herein alters or disrupts the nature of the heparan sulfate such that it inhibits heparan sulfate binding and heparan sulfate signaling. In some embodiments, the heparan sulfate inhibitor alters or disrupts the nature of the heparan sulfate such that it inhibits the binding, signaling, or a combination thereof of any lectin (including polypeptides) subject to heparan sulfate binding, signaling or a combination thereof, in the absence of a heparan sulfate inhibitor. In some embodiments, the lectin is, by way of non-limiting example, a growth factor. In specific embodiments, the growth factor is, by way of non-limiting example, fibroblast growth factor (FGF) or vascular endothelia growth factor (VEGF). In more specific embodiments, the growth factor is FGF.

In certain embodiments, the cell is present in an individual (e.g., a human) diagnosed with a disorder mediated by heparan sulfate. In certain instances, the disorder mediated by heparan sulfate is a cancer, a tumor, undesired angiogenesis (e.g., associated with cancer, diabetic blindness, age-related macular degeneration, rheumatoid arthritis, or psoriasis), insufficient angiogenesis (e.g., associated with coronary artery disease, stroke, or delayed wound healing), mucopolysaccharidosis, amyloidosis, a spinal cord injury, hypertriglyceridemia, inflammation, a wound, or the like. In some embodiments, the cell is present in a human diagnosed with cancer. In certain embodiments, the cell is present in an individual (e.g., a human) diagnosed with abnormal angiogenesis and/or undesired angiogenesis. In some embodiments, the cell is present in an individual (e.g., a human) diagnosed with a lysosomal storage disease (e.g., mucopolysaccharidosis (MPS)). In specific embodiments, the individual is diagnosed with MPS I, MPS II, or MPS III. In some specific embodiments, the individual is diagnosed with MPS I. In some specific embodiments, the individual is diagnosed with MPS II. In some specific embodiments, the individual is diagnosed with MPS III. In some embodiments, the cell is present in an individual (e.g., a human) diagnosed with amyloidosis, a spinal cord injury, hypertriglyceridemia, inflammation, or the like.

In some embodiments, the cell is present in an individual (e.g., a human) diagnosed with Alzheimer's disease, Parkinson's disease, Huntington's disease, spongiform encephalopathies (Creutzfeld-Jakob, Kuru, Mad Cow), diabetic amyloidosis, type-2 diabetes, Rheumatoid arthritis, juvenile chronic arthritis, Ankylosing spondylitis, psoriasis, psoriatic arthritis, adult still disease, Becet syndrome, familial Mediterranean fever, Crohn's disease, leprosy, osteomyelitis, tuberculosis, chronic bronchiectasis, Castleman disease, Hodgkin's disease, renal cell carcinoma, or carcinoma of the gut, lung or urogenital tract.

In some embodiments, the cell is present in an individual (e.g., human) diagnosed with pancreatic cancer, myeloma, ovarian cancer, hepatocellular cancer, breast cancer, colon carcinoma, or melanoma. In certain embodiments, the cell is a pancreatic cancer cell, myeloma cell, ovarian cancer cell, hepatocellular cancer cell, breast cancer cell, colon carcinoma cell, renal cell carcinoma, carcinoma of the gut, lung or urogenital tract, or melanoma cell.

In some embodiments, the cell is present in an individual (e.g., human) diagnosed with an infectious or viral disease including, by way of non-limiting example, herpes, diphtheria, papilloma virus, hepatitis, HIV, coronavirus, or adenovirus.

In certain embodiments, heparan sulfate modulators, and in specific embodiments heparan sulfate inhibitors, described herein are small molecule organic compounds. In certain instances, heparan sulfate modulators, and in specific embodiments heparan sulfate inhibitors, utilized herein are not polypeptides or carbohydrates. In some embodiments, a small molecule organic compounds has a molecular weight of less than 2,000 g/mol, less than 1,500 g/mol, less than 1,000 g/mol, or less than 500 g/mol.

In certain embodiments, provided herein is a method of treating a disorder mediated by heparan sulfate by administering to an individual (e.g., a human) in need thereof a therapeutically effective amount of any heparan sulfate modulator, and in specific embodiments heparan sulfate inhibitor, described herein. In specific embodiments, the heparan sulfate modulator or inhibitor is a modulator (e.g., inhibitor or promoter) of a heparan sulfate glycosyltransferase, a heparan sulfate sulfotransferase, or a heparan sulfate epimerase. In some specific embodiments, the heparan sulfate inhibitor is an inhibitor of a heparan sulfate glycosyltransferase. In some specific embodiments, the heparan sulfate inhibitor is an inhibitor of a heparan sulfate sulfotransferase. In some specific embodiments, the heparan sulfate inhibitor is an inhibitor of a heparan sulfate epimerase. In certain instances, the disorder mediated by heparan sulfate is a cancer, a tumor, undesired angiogenesis (e.g., associated with cancer, diabetic blindness, age-related macular degeneration, rheumatoid arthritis, or psoriasis), insufficient angiogenesis (e.g., associated with coronary artery disease, stroke, or delayed wound healing), mucopolysaccharidosis, amyloidosis, a spinal cord injury, hypertriglyceridemia, inflammation, a wound, or the like. In some embodiments, provided herein is a method of treating cancer by administering to an individual (e.g., a human) in need thereof a therapeutically effective amount of any heparan sulfate modulator, and in specific embodiments heparan sulfate inhibitor, described herein. In some embodiments, provided herein is a method of treating a tumor by administering to an individual (e.g., a human) in need thereof a therapeutically effective amount of any heparan sulfate modulator, and in specific embodiments heparan sulfate inhibitor, described herein. In some embodiments, provided herein is a method of treating undesired angiogenesis by administering to an individual (e.g., a human) in need thereof a therapeutically effective amount of any heparan sulfate modulator, and in specific embodiments heparan sulfate inhibitor, described herein. In some embodiments, provided herein is a method of treating a lysosomal storage disease (e.g., MPS) by administering to an individual (e.g., a human) in need thereof a therapeutically effective amount of any heparan sulfate modulator, and in specific embodiments heparan sulfate inhibitor, described herein. In some embodiments, provided herein is a method of treating a amyloidosis, a spinal cord injury, hypertriglyceridemia, and/or inflammation by administering to an individual (e.g., a human) in need thereof a therapeutically effective amount of any heparan sulfate modulator, and in specific embodiments heparan sulfate inhibitor, described herein.

In some embodiments, provided herein is a method of treating cancer by administering to an individual (e.g., human) a therapeutically effective amount of any heparan sulfate modulator, and in specific embodiments heparan sulfate inhibitor, described herein. In some embodiments, the cancer is, by way of non-limiting example, pancreatic cancer, myeloma, ovarian cancer, hepatocellular cancer, breast cancer, colon carcinoma, renal cell carcinoma, carcinoma of the gut, lung or urogenital tract, or melanoma. In some embodiments, provided herein is a method of treating pancreatic cancer in an individual in need thereof by administering to the individual a therapeutically effective selective heparan sulfate inhibitor, e.g., as described in any of Formulas III-VII, or FIG. 2. In some embodiments, provided herein is a method of treating ovarian cancer in an individual in need thereof by administering to the individual a therapeutically effective selective heparan sulfate inhibitor, e.g., as described in any of Formulas III-VII, or FIG. 2. In some embodiments, provided herein is a method of treating breast cancer in an individual in need thereof by administering to the individual a therapeutically effective selective heparan sulfate inhibitor, e.g., as described in any of Formulas III-VII, or FIG. 2. In some embodiments, provided herein is a method of treating lung cancer in an individual in need thereof by administering to the individual a therapeutically effective selective heparan sulfate inhibitor, e.g., as described in any of Formulas III-VII, or FIG. 2. In some embodiments, provided herein is a method of treating colon cancer in an individual in need thereof by administering to the individual a therapeutically effective selective heparan sulfate inhibitor, e.g., as described in any of Formulas III-VII, or FIG. 2. In some embodiments, provided herein is a method of treating prostate cancer in an individual in need thereof by administering to the individual a therapeutically effective selective heparan sulfate inhibitor, e.g., as described in any of Formulas III-VII, or FIG. 2. In some embodiments, provided herein is a method of treating leukemia in an individual in need thereof by administering to the individual a therapeutically effective selective heparan sulfate inhibitor, e.g., as described in any of Formulas III-VII, or FIG. 2. Provided in some embodiments herein is an adjuvant therapy for treating cancer, the method comprising administering (e.g., in combination with another chemotherapeutic agent) a selective heparan sulfate inhibitor, e.g., any compound of Formulas III-VII or FIG. 2. Provided in some embodiments herein is an adjuvant therapy for treating pancreatic cancer, the method comprising administering (e.g., in combination with another chemotherapeutic agent) a selective heparan sulfate inhibitor, e.g., any compound of Formulas III-VII or FIG. 2. Provided in some embodiments herein is an adjuvant therapy for treating ovarian cancer, the method comprising administering (e.g., in combination with another chemotherapeutic agent) a selective heparan sulfate inhibitor, e.g., any compound of Formulas III-VII or FIG. 2. Provided in some embodiments herein is an adjuvant therapy for treating breast cancer, the method comprising administering (e.g., in combination with another chemotherapeutic agent) a selective heparan sulfate inhibitor, e.g., any compound of Formulas III-VII or FIG. 2. Provided in some embodiments herein is an adjuvant therapy for treating lung cancer, the method comprising administering (e.g., in combination with another chemotherapeutic agent) a selective heparan sulfate inhibitor, e.g., any compound of Formulas III-VII or FIG. 2. Provided in some embodiments herein is an adjuvant therapy for treating colon cancer, the method comprising administering (e.g., in combination with another chemotherapeutic agent) a selective heparan sulfate inhibitor, e.g., any compound of Formulas III-VII or FIG. 2. Provided in some embodiments herein is an adjuvant therapy for treating prostrate cancer, the method comprising administering (e.g., in combination with another chemotherapeutic agent) a selective heparan sulfate inhibitor, e.g., any compound of Formulas III-VII or FIG. 2. Provided in some embodiments herein is an adjuvant therapy for treating leukemia, the method comprising administering (e.g., in combination with another chemotherapeutic agent) a selective heparan sulfate inhibitor, e.g., any compound of Formulas III-VII or FIG. 2.

In some embodiments, provided herein is a method of treating an infectious or viral disease by administering to an individual (e.g., human) a therapeutically effective amount of any heparan sulfate modulator, and in specific embodiments heparan sulfate inhibitor, described herein. In some embodiments, the infectious or viral disease includes, by way of non-limiting example, herpes, diphtheria, papilloma virus, hepatitis, HIV, coronavirus, or adenovirus.

In some embodiments, the treatment of amyloidosis includes the treatment of Alzheimer's disease, Parkinson's disease, type-2 diabetes, Huntington's disease, spongiform encephalopathies (Creutzfeld-Jakob, Kuru, Mad Cow), diabetic amyloidosis, Rheumatoid arthritis, juvenile chronic arthritis, Ankylosing spondylitis, psoriasis, psoriatic arthritis, adult still disease, Becet syndrome, familial Mediterranean fever, Crohn's disease, leprosy, osteomyelitis, tuberculosis, chronic bronchiectasis, Castleman disease, Hodgkin's disease, renal cell carcinoma, carcinoma of the gut, lung or urogenital tract.

Provided in certain embodiments herein is a process of inhibiting heparan sulfate function in a cell comprising contacting the cell with a selective modulator (e.g., with respect to other glycans, specifically GAGs) of heparan sulfate biosynthesis. In various embodiments, heparan sulfate biosynthesis, as used herein, includes, by way of non-limiting example, (1) inhibition of (a) heparan sulfate glycosylation; (b) heparan sulfate sulfation; (c) epimerization of uronic acid groups in heparan sulfate; (d) heparan sulfate phosphorylation and/or (e) deacetylation of GlcNAc groups in heparan sulfate; and/or (2) promotion of (a) heparan sulfate bond cleavage; (b) bond cleavage of the linker region connecting heparan sulfate to a core protein; (c) bond cleavage between heparan sulfate and the linker region; (d) desulfation (e.g., N-sulfation and/or O-sulfation) of heparan sulfate; (e) acetylation of GlcN groups in heparan sulfate; (f) deacetylation of GlcNAc groups in heparan sulfate; (g) heparan sulfate phosphorylation, and/or (h) epimerization of uronic acid groups in heparan sulfate. In some specific embodiments, heparan sulfate inhibitors described herein inhibit heparan sulfate glycosylation. In some specific embodiments, heparan sulfate inhibitors described herein inhibit heparan sulfate sulfation. In some specific embodiments, heparan sulfate inhibitors described herein inhibit epimerization of uronic acid groups in heparan sulfate. In some specific embodiments, heparan sulfate inhibitors described herein inhibit heparan sulfate phosphorylation. In some specific embodiments, heparan sulfate inhibitors described herein inhibit deacetylation of GlcNAc groups in heparan sulfate. In some specific embodiments, heparan sulfate inhibitors described herein promote heparan sulfate bond cleavage. In some specific embodiments, heparan sulfate inhibitors described herein promote bond cleavage of the linker region connecting heparan sulfate to a core protein. In some specific embodiments, heparan sulfate inhibitors described herein promote bond cleavage between heparan sulfate and the linker region. In some specific embodiments, heparan sulfate inhibitors described herein promote de-sulfation (e.g., N-sulfation and/or O-sulfation) of heparan sulfate. In some specific embodiments, heparan sulfate inhibitors described herein promote acetylation of GlcN groups in heparan sulfate. In some specific embodiments, heparan sulfate inhibitors described herein promote deacetylation of GlcNAc groups in heparan sulfate. In some specific embodiments, heparan sulfate inhibitors described herein promote heparan sulfate phosphorylation. In some specific embodiments, heparan sulfate inhibitors described herein promote epimerization of uronic acid groups in heparan sulfate. In specific embodiments, the modulator of heparan sulfate biosynthesis inhibits sulfation of heparan sulfate. In specific embodiments, the modulator of heparan sulfate biosynthesis promotes sulfation of heparan sulfate. In specific embodiments, the modulator of heparan sulfate biosynthesis inhibits epimerization of heparan sulfate. In specific embodiments, the modulator of heparan sulfate biosynthesis promotes epimerization of heparan sulfate.

In some embodiments, the modulator of heparan sulfate biosynthesis modulates (e.g., promotes or inhibits) glycosyltransferase. In some embodiments, the modulator of heparan sulfate glycosyltransferase inhibits the synthesis of the linkage region suitable for connecting heparan sulfate to a core protein, the initiation of heparan sulfate synthesis, the synthesis of heparan sulfate, or a combination thereof. In some embodiments, modulators of heparan sulfate biosynthesis modulate (e.g., promote or inhibit) one or more of a heparan sulfate xylosyltransfarase, a heparan sulfate galactosyltransferase, a heparan sulfate glucuronosyltransferase, a heparan sulfate N-acetylglucosamine transferase, or combinations thereof. In more specific embodiments, heparan sulfate modulator, and in specific embodiments heparan sulfate inhibitors, modulate (e.g., promote or inhibit) one or more of xylosyltransfarase I, xylosyltransfarase II, galactosyltransferase I, galactosyltransferase II, glucuronosyltransferase I, glucuronosyltransferase II, N-acetylglucosamine transferase I, N-acetylglucosamine transferase II, or a combination thereof.

In certain embodiments, modulators of heparan sulfate biosynthesis that modulate sulfation modulate one or more sulfotransferase. In specific embodiments, the sulfotransferase is, by way of non-limiting example, a modulator (e.g., inhibitor or promoter) of one or more of a heparan sulfate O-sulfotransferase, a heparan sulfate N-sulfotransferase, or a combination thereof. In more specific embodiments, the heparan sulfate modulator, and in specific embodiments heparan sulfate inhibitor, modulates (e.g., inhibits or promotes) a heparan sulfate O-sulfotransferase such as, by way of non-limiting example, one or more of a 6-O sulfotransferase (of a glucosylamine group), a 3-O sulfotransferase (of a glucosylamine group), a 2-O sulfotransferase (of a uronic acid moiety, e.g., glucuronic acid or iduronic acid), a 6-O sulfotransferase (of a galactose in the linkage tetrasaccharide), or a combination thereof. In some embodiments, modulators of heparan sulfate biosynthesis modulate 2-O phosphorylation of the xylose in the heparan sulfate linkage region.

In certain embodiments, the effective amount of the modulator of heparan sulfate biosynthesis alters or disrupts the nature (e.g., alters or disrupts the acetylation, sulfation, O-sulfation, the 2-O sulfation, the 3-O sulfation, the 6-O sulfation, the N-sulfation, concentration of heparan sulfate, emiperization of heparan sulfate, chain length of heparan sulfate, or a combination thereof) of heparan sulfate compared to endogenous heparan sulfate in an amount sufficient to alter or disrupt heparan sulfate binding, heparan sulfate signaling, or a combination thereof. In specific embodiments, the modulator of heparan sulfate biosynthesis alters or disrupts the nature of the heparan sulfate such that it inhibits heparan sulfate signaling. In other specific embodiments, the modulator of heparan sulfate biosynthesis alters or disrupts the nature of the heparan sulfate such that it inhibits heparan sulfate binding. In more specific embodiments, modulator of heparan sulfate biosynthesis alters or disrupts the nature of the heparan sulfate such that it inhibits heparan sulfate binding and heparan sulfate signaling. In some embodiments, modulator of heparan sulfate biosynthesis alters or disrupts the nature of the heparan sulfate such that it inhibits the binding, signaling, or a combination thereof of any lectin (including polypeptides) subject to heparan sulfate binding, signaling or a combination thereof, in the absence of a heparan sulfate inhibitor. In some embodiments, the lectin is, by way of non-limiting example, a growth factor. In specific embodiments, the growth factor is, by way of non-limiting example, fibroblast growth factor (FGF) or vascular endothelia growth factor (VEGF).

In certain embodiments, the selective modulator of heparan sulfate biosynthesis is a small molecule organic compound. In certain instances, selective modulator of heparan sulfate biosynthesis utilized herein is not a polypeptide or a carbohydrate. In certain embodiments, the small molecule organic compound has a molecular weight of less than 2,000 g/mol, less than 1,500 g/mol, less than 1,000 g/mol, or less than 500 g/mol.

Provided in certain embodiments herein is a method of treating cancer or neoplasia comprising administering a therapeutically effective amount of a heparan sulfate modulator, and in specific embodiments heparan sulfate inhibitor, to a patient in need thereof. In some embodiments, the heparan sulfate modulator, and in specific embodiments heparan sulfate inhibitor, reduces or inhibits tumor growth, reduces or inhibits angiogenesis, or a combination thereof. In certain embodiments, the heparan sulfate modulator, and in specific embodiments heparan sulfate inhibitor, is selective (as compared to other GAGs) modulator of heparan sulfate glycosylation (e.g., inhibits one or more heparan sulfate glycosyltransferase), modulator of heparan sulfate sulfation (e.g., inhibits or promotes one or more heparan sulfate sulfotransferase), selective modulator of heparan sulfate epimerization (e.g., inhibits or promotes one or more heparan sulfate epimerase). In various embodiments, heparan sulfate alters or reduces the function of heparan sulfate by one or more of the following non-limiting manners: (1) inhibition of (a) heparan sulfate glycosylation; (b) heparan sulfate sulfation; (c) epimerization of uronic acid groups in heparan sulfate; (d) heparan sulfate phosphorylation and/or (e) deacetylation of GlcNAc groups in heparan sulfate; and/or (2) promotion of (a) heparan sulfate bond cleavage; (b) bond cleavage of the linker region connecting heparan sulfate to a core protein; (c) bond cleavage between heparan sulfate and the linker region; (d) sulfation (e.g., N-sulfation and/or O-sulfation) of heparan sulfate; (e) acetylation of GlcN groups in heparan sulfate; (f) deacetylation of GlcNAc groups in heparan sulfate; (g) heparan sulfate phosphorylation, and/or (h) epimerization of uronic acid groups in heparan sulfate. In specific embodiments, the modulator of heparan sulfate biosynthesis inhibits sulfation of heparan sulfate. In specific embodiments, the modulator of heparan sulfate biosynthesis promotes sulfation of heparan sulfate. In specific embodiments, the modulator of heparan sulfate biosynthesis inhibits epimerization of heparan sulfate. In specific embodiments, the modulator of heparan sulfate biosynthesis promotes epimerization of heparan sulfate.

In some embodiments, the heparan sulfate modulator, and in specific embodiments heparan sulfate inhibitor, is a selective heparan sulfate modulator or inhibitor (as compared to the inhibition of the function of other GAGs), e.g., as described herein. In some embodiments, the selective heparan sulfate modulator, and in specific embodiments heparan sulfate inhibitor, is a modulator of (e.g., promotes one or more of, or inhibits one or more of) heparan sulfate glycosylation (e.g., modulates a heparan sulfate glycosyltransferase), heparan sulfate sulfation (e.g., modulates a heparan sulfate sulfotransferase), heparan sulfate epimerization (e.g., modulates a heparan sulfate epimerase), or a combination thereof.

In some embodiments, the heparan sulfate modulator, and in specific embodiments heparan sulfate inhibitor, modulates (e.g., promote or inhibit) glycosyltransferase. In some embodiments, the modulator, and in specific embodiments inhibitor, of a heparan sulfate glycosyltransferase inhibits the synthesis of the linkage region, the initiation of heparan sulfate synthesis, the synthesis of heparan sulfate, or a combination thereof. In some embodiments, heparan sulfate modulators or, and in specific embodiments heparan sulfate inhibitors, modulate (e.g., promote or inhibit) one or more of a heparan sulfate xylosyltransferase, a heparan sulfate galactosyltransferase, a heparan sulfate glucuronosyltransferase, a heparan sulfate N-acetylglucosamine transferase, or combinations thereof. In more specific embodiments, heparan sulfate modulators, and in specific embodiments heparan sulfate inhibitors, selectively modulate (e.g., promote or inhibit) one or more of xylosyltransfarase I, xylosyltransfarase II, galactosyltransferase I, galactosyltransferase II, glucuronosyltransferase I, glucuronosyltransferase II, N-acetylglucosamine transferase I, N-acetylglucosamine transferase II, or a combination thereof.

In certain embodiments, heparan sulfate modulators, and in specific embodiments heparan sulfate inhibitors, that modulate sulfation modulate one or more heparan sulfate sulfotransferase. In specific embodiments, the heparan sulfate sulfotransferase is, by way of non-limiting example, a modulator (e.g., inhibitor or promoter) of one or more of a heparan sulfate O-sulfotransferase, a heparan sulfate N-sulfotransferase, or a combination thereof. In more specific embodiments, the heparan sulfate modulator, and in specific embodiments heparan sulfate inhibitor, modulates (e.g., inhibits or promotes) a heparan sulfate O-sulfotransferase such as, by way of non-limiting example, one or more of a 6-O sulfotransferase (of a glucosylamine group), a 3-O sulfotransferase (of a glucosylamine group), a 2-O sulfotransferase (of a uronic acid moiety, e.g., glucuronic acid or iduronic acid), or a combination thereof.

In certain embodiments, the effective amount of heparan sulfate modulator, and in specific embodiments heparan sulfate inhibitor, alters or disrupts the nature (e.g., alters or disrupts the acetylation, sulfation, O-sulfation, the 2-O sulfation, the 3-O sulfation, the 6-O sulfation, the N-sulfation, concentration of heparan sulfate, emiperization of heparan sulfate, chain length of heparan sulfate, or a combination thereof) of heparan sulfate compared to endogenous heparan sulfate in an amount sufficient to alter or disrupt heparan sulfate binding, heparan sulfate signaling, or a combination thereof. In specific embodiments, the heparan sulfate inhibitor described herein alters or disrupts the nature of the heparan sulfate such that it inhibits heparan sulfate signaling. In other specific embodiments, the heparan sulfate inhibitor described herein alters or disrupts the nature of the heparan sulfate such that it inhibits heparan sulfate binding. In more specific embodiments, the heparan sulfate inhibitor described herein alters or disrupts the nature of the heparan sulfate such that it inhibits heparan sulfate binding and heparan sulfate signaling. In some embodiments, the heparan sulfate inhibitor alters or disrupts the nature of the heparan sulfate such that it inhibits the binding, signaling, or a combination thereof of any lectin (including polypeptides) subject to heparan sulfate binding, signaling or a combination thereof, in the absence of a heparan sulfate inhibitor. In some embodiments, the lectin is, by way of non-limiting example, a growth factor. In specific embodiments, the growth factor is, by way of non-limiting example, fibroblast growth factor (FGF) or vascular endothelia growth factor (VEGF).

In certain embodiments, heparan sulfate modulators, and in specific embodiments heparan sulfate inhibitors, described herein are small molecule organic compounds. In certain instances, heparan sulfate modulators, and in specific embodiments heparan sulfate inhibitors, utilized herein are not polypeptides or carbohydrates. In some embodiments, a small molecule organic compounds has a molecular weight of less than 2,000 g/mol, less than 1,500 g/mol, less than 1,000 g/mol, or less than 500 g/mol.

Provided in some embodiments herein is a method of treating a lysosomal storage disease comprising administering a therapeutically effective amount of a heparan sulfate modulator, or in specific embodiments heparan sulfate inhibitor, to an individual (e.g., a human) in need thereof. In certain embodiments, the heparan sulfate inhibitor is a selective (as compared to other GAGs) modulator, or in specific embodiments inhibitor, of heparan sulfate. In some embodiments, the selective heparan sulfate modulator, or in specific embodiments heparan sulfate inhibitor, is a selective modulator (e.g., inhibitor or promoter) of heparan sulfate glycosylation (e.g., of a heparan sulfate glycosyltransferase), a modulator (e.g., inhibitor or promoter) of heparan sulfate sulfation (e.g., of a heparan sulfate sulfotransferase), or a selective modulator (e.g., inhibitor or promoter) of heparan sulfate epimerization (e.g., of a heparan sulfate epimerase).

In specific embodiments, the lysosomal storage disease is, by way of non-limiting example, mucopolysaccharidosis (MPS). In more specific embodiments, the MPS is, by way of non-limiting example, MPS I, MPS II or MPS III. In specific embodiments, the MPS is MPS I. In some specific embodiments, the MPS is MPS II. In certain specific embodiments, the MPS is MPS III.

In various embodiments, the heparan sulfate inhibitor alters or disrupts the function of heparan sulfate by one or more of the following non-limiting manners: (I) inhibition of (a) heparan sulfate glycosylation; (b) heparan sulfate sulfation; (c) epimerization of uronic acid groups in heparan sulfate; (d) heparan sulfate phosphorylation and/or (e) deacetylation of GlcNAc groups in heparan sulfate; and/or (2) promotion of (a) heparan sulfate bond cleavage; (b) bond cleavage of the linker region connecting heparan sulfate to a core protein; (c) bond cleavage between heparan sulfate and the linker region; (d) sulfation (e.g., N-sulfation and/or O-sulfation) of heparan sulfate; (e) acetylation of GlcN groups in heparan sulfate; (f) deacetylation of GlcNAc groups in heparan sulfate; (g) heparan sulfate phosphorylation and/or (h) epimerization of uronic acid groups in heparan sulfate. In some specific embodiments, heparan sulfate inhibitors described herein inhibit heparan sulfate glycosylation. In some specific embodiments, heparan sulfate inhibitors described herein inhibit heparan sulfate sulfation. In some specific embodiments, heparan sulfate inhibitors described herein inhibit epimerization of uronic acid groups in heparan sulfate. In some specific embodiments, heparan sulfate inhibitors described herein inhibit heparan sulfate phosphorylation. In some specific embodiments, heparan sulfate inhibitors described herein inhibit deacetylation of GlcNAc groups in heparan sulfate. In some specific embodiments, heparan sulfate inhibitors described herein promote heparan sulfate bond cleavage. In some specific embodiments, heparan sulfate inhibitors described herein promote bond cleavage of the linker region connecting heparan sulfate to a core protein. In some specific embodiments, heparan sulfate inhibitors described herein promote bond cleavage between heparan sulfate and the linker region. In some specific embodiments, heparan sulfate inhibitors described herein promote de-sulfation (e.g., N-sulfation and/or O-sulfation) of heparan sulfate. In some specific embodiments, heparan sulfate inhibitors described herein promote acetylation of GlcN groups in heparan sulfate. In some specific embodiments, heparan sulfate inhibitors described herein promote deacetylation of GlcNAc groups in heparan sulfate. In some specific embodiments, heparan sulfate inhibitors described herein promote heparan sulfate phosphorylation. In some specific embodiments, heparan sulfate inhibitors described herein promote epimerization of uronic acid groups in heparan sulfate.

In some embodiments, the heparan sulfate modulator, or in specific embodiments heparan sulfate inhibitor, is a selective heparan sulfate modulator, or in specific embodiments heparan sulfate inhibitor, (as compared to the inhibition of the function, e.g., lectin binding, of other GAGs and/or extracellular glycans, such as N-linked glycans), e.g., as described herein. In some embodiments, the selective heparan sulfate modulator, or in specific embodiments heparan sulfate inhibitor, is a modulator of (e.g., promotes one or more of, or inhibits one or more of) heparan sulfate glycosylation (e.g., modulates a heparan sulfate glycosyltransferase), heparan sulfate sulfation (e.g., modulates a heparan sulfate sulfotransferase), heparan sulfate epimerization (e.g., modulates a heparan sulfate epimerase), or a combination thereof. In some specific embodiments, the heparan sulfate inhibitor inhibits heparan sulfate glycosylation (e.g., inhibits a heparan sulfate glycosyltransferase). In some specific embodiments, the heparan sulfate inhibitor inhibits heparan sulfate sulfation (e.g., inhibits a heparan sulfate sulfotransferase). In some specific embodiments, the heparan sulfate inhibitor inhibits heparan sulfate epimerization (e.g., inhibits a heparan sulfate epimerase).

In some embodiments, the heparan sulfate modulator, or in specific embodiments heparan sulfate inhibitor, modulates (e.g., promote or inhibit) glycosyltransferase. In some embodiments, the modulator, or in specific embodiments heparan sulfate inhibitor, of a heparan sulfate glycosyltransferase inhibits the synthesis of the linkage region, the initiation of heparan sulfate synthesis, the synthesis of heparan sulfate, or a combination thereof. In some embodiments, heparan sulfate modulator, or in specific embodiments heparan sulfate inhibitors, modulate (e.g., promote or inhibit) one or more of a heparan sulfate xylosyltransferase, a heparan sulfate galactosyltransferase, a heparan sulfate glucuronosyltransferase, a heparan sulfate N-acetylglucosamine transferase, or combinations thereof. In more specific embodiments, heparan sulfate modulators, or in specific embodiments heparan sulfate inhibitors, selectively modulate (e.g., promote or inhibit) one or more of xylosyltransfarase I, xylosyltransfarase II, galactosyltransferase I, galactosyltransferase II, glucuronosyltransferase I, glucuronosyltransferase II, N-acetylglucosamine transferase I, N-acetylglucosamine transferase II, or a combination thereof. In some specific embodiments, the heparan sulfate inhibitor inhibits xylosyltransfarase I. In some specific embodiments, the heparan sulfate inhibitor inhibits xylosyltransfarase II. In some specific embodiments, the heparan sulfate inhibitor inhibits galactosyltransferase I. In some specific embodiments, the heparan sulfate inhibitor inhibits galactosyltransferase II. In some specific embodiments, the heparan sulfate inhibitor inhibits glucuronosyltransferase I. In some specific embodiments, the heparan sulfate inhibitor inhibits glucuronosyltransferase II. In some specific embodiments, the heparan sulfate inhibitor inhibits N-acetylglucosamine transferase I. In some specific embodiments, the heparan sulfate inhibitor inhibits N-acetylglucosamine transferase II.

In certain embodiments, heparan sulfate modulators, or in specific embodiments heparan sulfate inhibitors, that modulate sulfation modulate one or more heparan sulfate sulfotransferase. In specific embodiments, the heparan sulfate sulfotransferase is, by way of non-limiting example, a modulator (e.g., inhibitor or promoter) of one or more of a heparan sulfate O-sulfotransferase, a heparan sulfate N-sulfotransferase, or a combination thereof. In more specific embodiments, the heparan sulfate inhibitor modulates (e.g., inhibits or promotes) a heparan sulfate O-sulfotransferase such as, by way of non-limiting example, one or more of a 6-O sulfotransferase (of a glucosylamine group), a 3-O sulfotransferase (of a glucosylamine group), a 2-O sulfotransferase (of a uronic acid moiety, e.g., glucuronic acid or iduronic acid), a 6-O sulfotransferase of galactose, or a combination thereof. In some specific embodiments, the heparan sulfate inhibitor inhibits heparan sulfate O-sulfotransferase. In some specific embodiments, the heparan sulfate inhibitor inhibits a heparan sulfate N-sulfotransferase. In some specific embodiments, the heparan sulfate inhibitor inhibits a heparan sulfate O-sulfotransferase. In some specific embodiments, the heparan sulfate inhibitor inhibits a 6-O sulfotransferase (of a glucosylamine group). In some specific embodiments, the heparan sulfate inhibitor inhibits a 3-O sulfotransferase (of a glucosylamine group). In some specific embodiments, the heparan sulfate inhibitor inhibits a 2-O sulfotransferase (of a uronic acid moiety, e.g., glucuronic acid or iduronic acid). In some specific embodiments, the heparan sulfate inhibitor inhibits a 6-O sulfotransferase of galactose.

In certain embodiments, the effective amount of heparan sulfate modulator, or in specific embodiments heparan sulfate inhibitor, alters or disrupts the nature (e.g., alters or disrupts the acetylation, sulfation, O-sulfation, the 2-O sulfation, the 3-O sulfation, the 6-O sulfation, the N-sulfation, concentration of heparan sulfate, emiperization of heparan sulfate, chain length of heparan sulfate, phosphorylation, or a combination thereof) of heparan sulfate compared to endogenous heparan sulfate in an amount sufficient to alter or disrupt heparan sulfate binding, heparan sulfate signaling, or a combination thereof. In specific embodiments, the heparan sulfate modulator, or in specific embodiments heparan sulfate inhibitor, described herein alters or disrupts the nature of the heparan sulfate such that it inhibits heparan sulfate signaling. In other specific embodiments, the heparan sulfate modulator, or in specific embodiments heparan sulfate inhibitor, described herein alters or disrupts the nature of the heparan sulfate such that it inhibits heparan sulfate binding. In more specific embodiments, the heparan sulfate modulator, or in specific embodiments heparan sulfate inhibitor, described herein alters or disrupts the nature of the heparan sulfate such that it inhibits heparan sulfate binding and heparan sulfate signaling. In some embodiments, the heparan sulfate modulator, or in specific embodiments heparan sulfate inhibitor, alters or disrupts the nature of the heparan sulfate such that it inhibits the binding, signaling, or a combination thereof of any lectin (including polypeptides) subject to heparan sulfate binding, signaling or a combination thereof, in the absence of a heparan sulfate inhibitor. In some embodiments, the lectin is, by way of non-limiting example, a growth factor. In specific embodiments, the growth factor is, by way of non-limiting example, fibroblast growth factor (FGF) or vascular endothelia growth factor (VEGF).

In certain embodiments, heparan sulfate modulators, or in specific embodiments heparan sulfate inhibitors, described herein are small molecule organic compounds. In certain instances, heparan sulfate modulators, or in specific embodiments heparan sulfate inhibitors, utilized herein are not polypeptides or carbohydrates. In some embodiments, a small molecule organic compounds has a molecular weight of less than 2,000 g/mol, less than 1,500 g/mol, less than 1,000 g/mol, or less than 500 g/mol. In specific embodiments, the heparan sulfate inhibits has a molecular weight of less than 2,000 g/mol. In specific embodiments, the heparan sulfate inhibits has a molecular weight of less than 1,500 g/mol. In specific embodiments, the heparan sulfate inhibits has a molecular weight of less than 1,000 g/mol. In specific embodiments, the heparan sulfate inhibits has a molecular weight of less than 500 g/mol.

Provided in some embodiments herein is a method of treating hyperheparansulfatemia comprising administering a therapeutically effective amount of a heparan sulfate modulator, or in specific embodiments heparan sulfate inhibitor, to an individual (e.g., a human) in need thereof. In certain embodiments, the heparan sulfate modulator is a selective (as compared to other GAGs) inhibitor of heparan sulfate. In some embodiments, the selective heparan sulfate modulator, or in specific embodiments heparan sulfate inhibitor, is a selective modulator (e.g., inhibitor or promoter) of heparan sulfate glycosylation (e.g., of a heparan sulfate glycosyltransferase), a modulator (e.g., inhibitor or promoter) of heparan sulfate sulfation (e.g., of a heparan sulfate sulfotransferase), or a selective modulator (e.g., inhibitor or promoter) of heparan sulfate epimerization (e.g., of a heparan sulfate epimerase). In some embodiments, the therapeutically effective amount lowers the concentration of heparan sulfate (e.g., functional and/or endogenous-type heparan sulfate) by at least about 10% from pre-treatment levels. In some embodiments, the therapeutically effective amount lowers the concentration of heparan sulfate by at least about 20% from pre-treatment levels. In some embodiments, the therapeutically effective amount lowers the concentration of heparan sulfate by at least about 30% from pre-treatment levels. In some embodiments, the therapeutically effective amount lowers the concentration of heparan sulfate by at least about 40% from pre-treatment levels. In some embodiments, the therapeutically effective amount lowers the concentration of heparan sulfate by at least about 50% from pre-treatment levels. In some embodiments, the therapeutically effective amount lowers the concentration of heparan sulfate by at least about 60% from pre-treatment levels. In some embodiments, the therapeutically effective amount lowers the concentration of heparan sulfate by at least about 70% from pre-treatment levels. In some embodiments, the therapeutically effective amount lowers the concentration of heparan sulfate by at least about 80% from pre-treatment levels. In some embodiments, the therapeutically effective amount lowers the concentration of heparan sulfate by at least about 90% from pre-treatment levels.

Hyperheparansulfatemia is characterized by an elevated level of heparan sulfate (i.e., functional heparan sulfate) in an individual, the blood of an individual, in a specific organ or tissue, or the urine of an individual. A normal level of heparan sulfate can be determined by measuring the amount of heparan sulfate in an individual (or a specific tissue or organ of an individual), in the blood, or in the urine of an individual and determining a normal range. In certain embodiments, heparansulfatemia is characterized by the presence of various symptoms including, by way of non-limiting example, cancer (e.g., pancreatic cancer, ovarian cancer, hepatocellular cancer, breast cancer, colon carcinoma, or melanoma), tumor growth, angiogenesis, lysosomal storage disease, hypertriglyceridemia, amyloidosis, inflammation, or the like. In some instances, hyperheparansulfatemia can be diagnosed by measuring the level of heparan sulfate present in an individual, e.g., following the onset of one or more of the symptoms of hyperheparansulfatemia.

In specific embodiments, the lysosomal storage disease is, by way of non-limiting example, mucopolysaccharidosis (MPS). In more specific embodiments, the MPS is, by way of non-limiting example, MPS I, MPS II or MPS III.

In various embodiments, the heparan sulfate inhibitor alters or disrupts the function of heparan sulfate by one or more of the following non-limiting manners: (I) inhibition of (a) heparan sulfate glycosylation; (b) heparan sulfate sulfation; (c) epimerization of uronic acid groups in heparan sulfate; (d) heparan sulfate phosphorylation and/or (e) deacetylation of GlcNAc groups in heparan sulfate; and/or (2) promotion of (a) heparan sulfate bond cleavage; (b) bond cleavage of the linker region connecting heparan sulfate to a core protein; (c) bond cleavage between heparan sulfate and the linker region; (d) sulfation (e.g., N-sulfation and/or O-sulfation) of heparan sulfate; (e) acetylation of GlcN groups in heparan sulfate; (f) deacetylation of GlcNAc groups in heparan sulfate; (g) heparan sulfate phosphorylation, (h) and/or epimerization of uronic acid groups in heparan sulfate. In some specific embodiments, heparan sulfate inhibitors described herein inhibit heparan sulfate glycosylation. In some specific embodiments, heparan sulfate inhibitors described herein inhibit heparan sulfate sulfation. In some specific embodiments, heparan sulfate inhibitors described herein inhibit epimerization of uronic acid groups in heparan sulfate. In some specific embodiments, heparan sulfate inhibitors described herein inhibit heparan sulfate phosphorylation. In some specific embodiments, heparan sulfate inhibitors described herein inhibit deacetylation of GlcNAc groups in heparan sulfate. In some specific embodiments, heparan sulfate inhibitors described herein promote heparan sulfate bond cleavage. In some specific embodiments, heparan sulfate inhibitors described herein promote bond cleavage of the linker region connecting heparan sulfate to a core protein. In some specific embodiments, heparan sulfate inhibitors described herein promote bond cleavage between heparan sulfate and the linker region. In some specific embodiments, heparan sulfate inhibitors described herein promote de-sulfation (e.g., N-sulfation and/or O-sulfation) of heparan sulfate. In some specific embodiments, heparan sulfate inhibitors described herein promote acetylation of GlcN groups in heparan sulfate. In some specific embodiments, heparan sulfate inhibitors described herein promote deacetylation of GlcNAc groups in heparan sulfate. In some specific embodiments, heparan sulfate inhibitors described herein promote heparan sulfate phosphorylation. In some specific embodiments, heparan sulfate inhibitors described herein promote epimerization of uronic acid groups in heparan sulfate.

In some embodiments, the heparan sulfate modulator, or in specific embodiments heparan sulfate inhibitor, is a selective heparan sulfate modulator, or in specific embodiments heparan sulfate inhibitor, (as compared to the inhibition of the function of other GAGs), e.g., as described herein. In some embodiments, the selective heparan sulfate modulator, or in specific embodiments heparan sulfate inhibitor, is a modulator of (e.g., promotes one or more of, or inhibits one or more of) heparan sulfate glycosylation (e.g., modulates a heparan sulfate glycosyltransferase), heparan sulfate sulfation (e.g., modulates a heparan sulfate sulfotransferase), heparan sulfate epimerization (e.g., modulates a heparan sulfate epimerase), or a combination thereof.

In some embodiments, the heparan sulfate modulator, or in specific embodiments heparan sulfate inhibitor, modulates (e.g., promote or inhibit) glycosyltransferase. In some embodiments, the inhibitor of a heparan sulfate glycosyltransferase inhibits the synthesis of the linkage region, the initiation of heparan sulfate synthesis, the synthesis of heparan sulfate, or a combination thereof. In some embodiments, heparan sulfate modulators, or in specific embodiments heparan sulfate inhibitors, modulate (e.g., promote or inhibit) one or more of a heparan sulfate xylosyltransferase, a heparan sulfate galactosyltransferase, a heparan sulfate glucuronosyltransferase, a heparan sulfate N-acetylglucosamine transferase, or combinations thereof. In more specific embodiments, heparan sulfate modulators, or in specific embodiments heparan sulfate inhibitors, selectively modulate (e.g., promote or inhibit) one or more of xylosyltransfarase I, xylosyltransfarase II, galactosyltransferase I, galactosyltransferase II, glucuronosyltransferase I, glucuronosyltransferase II, N-acetylglucosamine transferase I, N-acetylglucosamine transferase II, or a combination thereof.

In certain embodiments, heparan sulfate modulators, or in specific embodiments heparan sulfate inhibitors, that modulate (e.g., promote or inhibit) sulfation modulate one or more heparan sulfate sulfotransferase. In specific embodiments, the heparan sulfate sulfotransferase is, by way of non-limiting example, a modulator (e.g., inhibitor or promoter) of one or more of a heparan sulfate O-sulfotransferase, a heparan sulfate N-sulfotransferase, or a combination thereof. In more specific embodiments, the heparan sulfate modulator, or in specific embodiments heparan sulfate inhibitor, modulates (e.g., inhibits or promotes) a heparan sulfate O-sulfotransferase such as, by way of non-limiting example, one or more of a 6-O sulfotransferase (of a glucosylamine group), a 3-O sulfotransferase (of a glucosylamine group), a 2-O sulfotransferase (of a uronic acid moiety, e.g., glucuronic acid or iduronic acid), or a combination thereof.

In certain embodiments, the effective amount of heparan sulfate inhibitor alters or disrupts the nature (e.g., alters or disrupts the acetylation, sulfation, O-sulfation, the 2-O sulfation, the 3-O sulfation, the 6-O sulfation, the N-sulfation, concentration of heparan sulfate, emiperization of heparan sulfate, chain length of heparan sulfate, or a combination thereof) of heparan sulfate compared to endogenous heparan sulfate in an amount sufficient to alter or disrupt heparan sulfate binding, heparan sulfate signaling, or a combination thereof. In specific embodiments, the heparan sulfate inhibitor described herein alters or disrupts the nature of the heparan sulfate such that it inhibits heparan sulfate signaling. In other specific embodiments, the heparan sulfate inhibitor described herein alters or disrupts the nature of the heparan sulfate such that it inhibits heparan sulfate binding. In more specific embodiments, the heparan sulfate inhibitor described herein alters or disrupts the nature of the heparan sulfate such that it inhibits heparan sulfate binding and heparan sulfate signaling. In some embodiments, the heparan sulfate inhibitor alters or disrupts the nature of the heparan sulfate such that it inhibits the binding, signaling, or a combination thereof of any lectin (including polypeptides) subject to heparan sulfate binding, signaling or a combination thereof, in the absence of a heparan sulfate inhibitor. In some embodiments, the lectin is, by way of non-limiting example, a growth factor. In specific embodiments, the growth factor is, by way of non-limiting example, fibroblast growth factor (FGF) or vascular endothelia growth factor (VEGF).

Provided in certain embodiments herein is a method of reducing the mean or median sulfation of heparan sulfate in (or endogenous to) an individual comprising administering a therapeutically effective amount of a heparan sulfate inhibitor to a patient in need thereof. In certain embodiments, the method of reducing the mean or median sulfation of heparan sulfate in (or endogenous to) an individual is suitable for treating heparansulfatemia or the symptoms thereof. In certain embodiments, the heparan sulfate inhibitor is selective (as compared to other GAGs and/or extracellular glycans, such as N-linked glycan) modulator of heparan sulfate glycosylation (e.g., inhibits one or more heparan sulfate glycosyltransferase), modulator of heparan sulfate sulfation (e.g., inhibits or promotes one or more heparan sulfate sulfotransferase), selective modulator of heparan sulfate epimerization (e.g., inhibits or promotes one or more heparan sulfate epimerase). In various embodiments, heparan sulfate alters or reduces the function of heparan sulfate by one or more of the following non-limiting manners: (1) inhibition of (a) heparan sulfate glycosylation; (b) heparan sulfate sulfation; (c) epimerization of uronic acid groups in heparan sulfate; (d) heparan sulfate phosphorylation and/or (e) deacetylation of GlcNAc groups in heparan sulfate; and/or (2) promotion of (a) heparan sulfate bond cleavage; (b) bond cleavage of the linker region connecting heparan sulfate to a core protein; (c) bond cleavage between heparan sulfate and the linker region; (d) sulfation (e.g., N-sulfation and/or O-sulfation) of heparan sulfate; (e) acetylation of GlcN groups in heparan sulfate; (f) deacetylation of GlcNAc groups in heparan sulfate; (g) heparan sulfate phosphorylation, and/or (h) epimerization of uronic acid groups in heparan sulfate. In specific embodiments, the modulator of heparan sulfate biosynthesis inhibits sulfation of heparan sulfate. In specific embodiments, the modulator of heparan sulfate biosynthesis promotes sulfation of heparan sulfate. In specific embodiments, the modulator of heparan sulfate biosynthesis inhibits epimerization of heparan sulfate. In specific embodiments, the modulator of heparan sulfate biosynthesis promotes epimerization of heparan sulfate.

In certain embodiments, heparan sulfate modulators, or in specific embodiments heparan sulfate inhibitors, described herein are small molecule organic compounds. In certain instances, heparan sulfate modulator, or in specific embodiments heparan sulfate inhibitors, utilized herein are not polypeptides or carbohydrates. In some embodiments, a small molecule organic compounds has a molecular weight of less than 2,000 g/mol, less than 1,500 g/mol, less than 1,000 g/mol, or less than 500 g/mol.

### Glycans and Proteoglycans

Provided in certain embodiments herein is a heparan sulfate proteoglycan comprising a core protein covalently linked to at least one heparan sulfate, wherein the at least one heparan sulfate comprises a plurality of glucosamine groups, and wherein less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, about 0.1% to about 20%, about 1% to about 19%, or about 1% to about 15% of the plurality of glucosamine groups are N-sulfated. In specific embodiments, the core protein is a human protein. In certain embodiments provided herein is a population of heparan sulfate proteoglycans present in a human tissue, each heparan sulfate proteoglycan comprising a core protein covalently linked to at least one heparan sulfate, wherein each heparan sulfate comprises a plurality of glucosamine groups, and wherein less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, about 0.1% to about 20%, about 1% to about 19%, or about 1% to about 15% of the glucosamine groups are N-sulfated. In some embodiments, provided herein is a heparan sulfate proteoglycan present in human liver tissue and comprising a core protein covalently linked to at least one heparan sulfate, the heparan sulfate comprising a plurality of glucosamine groups, and wherein less than 50%, less than 45%, less than 40%, less than 35%, less than 30%, less than 25%, less than 20%, or less than 15% of the plurality of glucosamine groups are N-sulfated. In certain embodiments, provided herein is a plurality of heparan sulfate proteoglycans present in human liver tissue, wherein each heparan sulfate proteoglycan comprises a core protein covalently linked to at least one heparan sulfate, wherein each heparan sulfate comprises a plurality of glucosamine groups, and wherein less than 50%, less than 45%, less than 40%, less than 35%, less than 30%, less than 25%, less than 20%, or less than 15% of the glucosamine groups are N-sulfated. In certain embodiments, provided is a population of heparan sulfate within human liver tissue, the population of heparan sulfate comprising a plurality of glucosamine groups, wherein less than 50%, less than 45%, less than 40%, less than 35%, less than 30%, less than 25%, less than 20%, or less than 15% of the plurality of glucosamine groups in the population of heparan sulfate are N-sulfated. In some embodiments, provided herein is human liver tissue comprising a population of heparan sulfate (e.g., including free heparan sulfate and/or heparan sulfate proteoglycan), each heparan sulfate comprising a plurality of glucosamine groups, the population of heparan sulfate having (e.g., on average) less than 50%, less than 45%, less than 40%, less than 35%, less than 30%, less than 25%, less than 20%, or less than 15% of the glucosamine groups N-sulfated. In certain instances, human liver tissue comprises a whole human liver and/or portions thereof. In some instances, the identification of characteristics of the heparan sulfate within a whole human liver can be inferred from a portion of the human liver (i.e., a tissue sample taken from the human liver).

Provided in some embodiments herein is a heparan sulfate proteoglycan comprising a core protein covalently linked to at least one heparan sulfate, wherein the at least one heparan sulfate comprises a plurality of glucosamine groups, and wherein less than 10%, less than 9%, less than 8%, less than 7%, less than 6%, less than 5%, less than 4%, less than 3%, about 0.1% to about 10%, about 1% to about 9%, or about 1% to about 5% of the plurality of glucosamine groups are 6-O sulfated. In specific embodiments, the core protein is a human protein. In certain embodiments provided herein is a population of heparan sulfate proteoglycans present in a human tissue, each heparan sulfate proteoglycan comprising a core protein covalently linked to at least one heparan sulfate, wherein each heparan sulfate comprises a plurality of glucosamine groups, and wherein less than 10%, less than 9%, less than 8%, less than 7%, less than 6%, less than 5%, less than 4%, less than 3%, about 0.1% to about 10%, about 1% to about 9%, or about 1% to about 5% of the glucosamine groups are 6-O sulfated. In some embodiments, provided herein is a heparan sulfate proteoglycan present in human liver tissue and comprising a core protein covalently linked to at least one heparan sulfate, the heparan sulfate comprising a plurality of glucosamine groups, and wherein less than 40%, less than 35%, less than 30%, less than 25%, less than 20%, or less than 15% of the plurality of glucosamine groups are 6-O sulfated. In certain embodiments, provided herein is a plurality of heparan sulfate proteoglycans present in human liver tissue, wherein each heparan sulfate proteoglycan comprises a core protein covalently linked to at least one heparan sulfate, wherein each heparan sulfate comprises a plurality of glucosamine groups, and wherein less than 40%, less than 35%, less than 30%, less than 25%, less than 20%, or less than 15% of the glucosamine groups are 6-O sulfated. In certain embodiments, provided is a population of heparan sulfate within human liver tissue, the population of heparan sulfate comprising a plurality of glucosamine groups, wherein less than 40%, less than 35%, less than 30%, less than 25%, less than 20%, or less than 15% of the plurality of glucosamine groups in the population of heparan sulfate are 6-O sulfated. In some embodiments, provided herein is human liver tissue comprising a population of heparan sulfate (e.g., including free heparan sulfate and/or heparan sulfate proteoglycan), each heparan sulfate comprising a plurality of glucosamine groups, the population of heparan sulfate having (e.g., on average) less than 40%, less than 35%, less than 30%, less than 25%, less than 20%, or less than 15% of the glucosamine groups 6-O sulfated.

Provided in some embodiments herein is a heparan sulfate proteoglycan comprising a core protein covalently linked to at least one heparan sulfate, wherein the at least one heparan sulfate comprises a plurality of uronic acid groups (e.g., glucuronic and iduronic acid groups), and wherein less than 10%, less than 9%, less than 8%, less than 7%, less than 6%, less than 5%, less than 4%, less than 3%, about 0.1% to about 10%, about 1% to about 9%, or about 1% to about 5% of the plurality of uronic acid groups are 2-O sulfated. In specific embodiments, the core protein is a human protein. In certain embodiments provided herein is a population of heparan sulfate proteoglycans present in a human tissue, each heparan sulfate proteoglycan comprising a core protein covalently linked to at least one heparan sulfate, wherein each heparan sulfate comprises a plurality of glucosamine groups, and wherein less than 10%, less than 9%, less than 8%, less than 7%, less than 6%, less than 5%, less than 4%, less than 3%, about 0.1% to about 10%, about 1% to about 9%, or about 1% to about 5% of the uronic acid groups are 2-O sulfated. In some embodiments, provided herein is a heparan sulfate proteoglycan present in human liver tissue and comprising a core protein covalently linked to at least one heparan sulfate, the heparan sulfate comprising a plurality of uronic acid groups, and wherein less than 28%, less than 25%, less than 20%, less than 15%, less than 10%, or less than 5% of the plurality of uronic acid groups are 2-O sulfated. In certain embodiments, provided herein is a plurality of heparan sulfate proteoglycans present in human liver tissue, wherein each heparan sulfate proteoglycan comprises a core protein covalently linked to at least one heparan sulfate, wherein each heparan sulfate comprises a plurality of glucosamine groups, and wherein less than 28%, less than 25%, less than 20%, less than 15%, less than 10%, or less than 5% of the uronic acid groups are 2-O sulfated. In certain embodiments, provided is a population of heparan sulfate within human liver tissue, the population of heparan sulfate comprising a plurality of uronic acid groups, wherein less than 28%, less than 25%, less than 20%, less than 15%, less than 10%, or less than 5% of the plurality of uronic acid groups in the population of heparan sulfate are 2-O sulfated. In some embodiments, provided herein is human liver tissue comprising a population of heparan sulfate (e.g., including free heparan sulfate and/or heparan sulfate proteoglycan), each heparan sulfate comprising a plurality of glucosamine groups, the population of heparan sulfate having (e.g., on average) less than 28%, less than 25%, less than 20%, less than 15%, less than 10%, or less than 5% of the uronic groups 2-O sulfated.

In some embodiments, a heparan sulfate proteoglycan or heparan sulfate described herein is a human heparan sulfate proteoglycan or human heparan sulfate.

TABLE 2
Unique HS Compositions (via inhibition of sulfation)

| | Tissue or | NA | NS | 2S | 6S | Ave sulfation/ |
|---|---|---|---|---|---|---|
| Mammal | Organ | (mol. %) | (mol. %) | (mol. %) | (mol. %) | disaccharide |
| Bovine | Kidney | >72, 75, 80, 90 | <28, 25, 20, 10 | <10, 8, 6, 4 | <20, 15, 10, 5 | <0.6, 0.55, 0.5, 0.4 |
| Bovine | Brain | >66, 70, 75, 85 | <34, 30, 25, 15 | <6, 4, 2, 1 | <25, 20, 15, 10 | <0.65, 0.6, 0.55, 0.45 |
| Bovine | Kidney | >72, 75, 80, 90 | <28, 25, 20, 10 | <8, 6, 4, 2 | <35, 30, 25, 20 | <0.7, 0.65, 0.6, 0.5 |
| Bovine | Lung | >57, 60, 65, 75 | <43, 40, 35, 25 | <4, 2, 1, 0.5 | <41, 40, 35, 30 | <0.85, 0.8, 0.75, 0.65 |
| Pig | Liver | >59, 60, 65, 75 | <42, 40, 35, 25 | <25, 20, 15, 10 | <37, 35, 30, 25 | <1.0, 0.95, 0.9, 0.8 |
| Pig | Intestine | >53, 55, 60, 70 | <47, 45, 40, 30 | <16, 15, 10, 5 | <24, 20, 15, 10 | <0.85, 0.8, 0.75, 0.65 |
| Camel | Lung | >30, 35, 40, 50 | <70, 65, 60, 50 | <44, 40, 35, 30 | <58, 55, 50, 45 | <1.7, 1.6, 1.5, 1.4 |
| Camel | Intestine | >40, 45, 50, 60 | <60, 55, 50, 40 | <27, 25, 20, 15 | <46, 45, 40, 30 | <1.3, 1.2, 1.1, 1.0 |
| Camel | Liver | >47, 50, 55, 65 | <53, 50, 45, 35 | <18, 15, 10, 5 | <43, 40, 35, 30 | <1.1, 1.0, 0.9, 0.8 |

TABLE 3
Table 3: Unique HS Compositions (via promotion of sulfation)

| | Tissue or | NA | NS | 2S | 6S | Ave sulfation/ |
|---|---|---|---|---|---|---|
| Mammal | Organ | (mol. %) | (mol. %) | (mol. %) | (mol. %) | disaccharide |
| Bovine | Kidney | <70, 65, 60, 50 | >29, 30, 35, 45 | >11, 15, 20, 25 | >21, 25, 30, 35 | >0.65, 0.7, 0.75, 0.8 |
| Bovine | Brain | <65, 60, 55, 45 | >35, 40, 45, 55 | >7, 10, 15, 20 | >26, 30, 35, 40 | >0.70, 0.75, 0.80, 0.85 |
| Bovine | Kidney | <71, 65, 60, 50 | >29, 30, 35, 45 | >9, 10, 15, 20 | >36, 40, 45, 50 | >0.75, 0.80, 0.85, 0.9 |
| Bovine | Lung | <56, 50, 45, 40 | >44, 45, 50, 60 | >5, 10, 15, 20 | >42, 45, 50, 55 | >0.90, 0.95, 1.0, 1.05 |
| Pig | Liver | <58, 55, 50, 40 | >43, 45, 50, 60 | >26, 30, 35, 40 | >38, 40, 45, 50 | >1.1, 1.2, 1.3, 1.4 |
| Pig | Intestine | <52.50, 45, 35 | >48, 50, 55, 65 | >17, 20, 25, 30 | >25, 30, 35, 40 | >0.9, 0.95, 1.0, 1.05 |
| Camel | Lung | <29, 25, 20, 10 | >71, 75, 80, 90 | >45, 50, 55, 60 | >59, 60, 65, 70 | >1.75, 1.8, 1.9, 2.0 |
| Camel | Intestine | <39, 35, 30, 20 | >61, 65, 70, 80 | >28, 30, 35, 40 | >47, 50, 55, 60 | >1.4, 1.5, 1.6, 1.7 |
| Camel | Liver | <46, 40, 35, 25 | >54, 55, 60, 70 | >19, 20, 25, 30 | >44, 45, 50, 55 | >1.2, 1.3, 1.4, 1.5 |

In certain embodiments, provided herein is a population of heparan sulfate comprising a plurality of glucosamine and uronic acid groups within mammalian cells, tissue or organs. In specific embodiments, the population of heparan sulfate possesses a specific amount of 6-O sulfation of the plurality of glucosamine groups, N-sulfation of the glucosamine groups, and/or 2-O sulfation of the plurality of uronic acid groups. In some embodiments, these amounts are less than the amounts present in endogenous tissue (i.e., tissue that has not been treated with a heparan sulfate described herein). In illustrative examples, heparan sulfate populations possess any one or more of the characteristics set forth in Table 2 or Table 3.

Provided in certain embodiments herein is a compound having the structure: H(XY)ₙ-GlcNAcα4GlcAβ3Galβ3Galβ4Xylβ-O-L-Ser-R₂. In specific embodiments, the compound is a human heparan sulfate compound. In more specific embodiments, the compound is a human liver heparan sulfate compound. In some embodiments, each R is independently H or at least one amino acid. In certain embodiments, n is 1-300. In some embodiments, the Xylβ group is optionally 2-O phosphorylated (PO₃R⁵₂). In certain embodiments, each Galβ group is independently optionally 6-O sulfated (SO₃R⁵). In some embodiments, each X is:

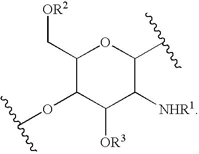

In certain embodiments, each R¹ is independently H, COCH₃, or SO₃R⁵. In some embodiments, each R² is independently H, or SO₃R⁵. In certain embodiments, each R³ is independently H, or SO₃R⁵. In some embodiments, each Y is:

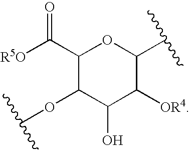

In certain embodiments, each R⁴ is independently H, or SO₃R⁵. In some embodiments, each R⁵ is independently selected from H and a negative charge. In some embodiments, provided is a physiologically acceptable salts of the compound.

In some embodiments, the compound has one or more of the following ratios:
a. R¹═SO₃R⁵ to R¹═COCH₃ is about 0:1 to about 0.7:1, about 0:1 to about 0.5:1, about 0:1 to about 0.3:1, about 0:1 to about 0.25:1, about 0:1 to about 0.2:1, about 0:1 to about 0.15:1, or about 0:1 to about 0.1:1;
b. R⁴═SO₃R¹ to R¹═SO₃R⁵ is about 0:1 to about 0.7:1, about 0:1 to about 0.5:1, about 0:1 to about 0.3:1, about 0:1 to about 0.25:1, about 0:1 to about 0.2:1, about 0:1 to about 0.15:1, about 0:1 to about 0.1:1, or >0.8:1;
c. R²═SO₃R⁵ to R¹═SO₃R⁵ is about 0:1 to about 0.7:1, about 0:1 to about 0.6:1, about 0:1 to about 0.55:1, about 0:1 to about 0.5:1, about 0:1 to about 0.45:1, about 0:1 to about 0.4:1, about 0:1 to about 0.3:1, about 0:1 to about 0.2:1, about 0:1 to about 0.1:1, or >11:1;
d. R⁴═SO₃R¹ to R²═SO₃R⁵ is about 0:1 to about 0.7:1, about 0:1 to about 0.6:1, about 0:1 to about 0.55:1, about 0:1 to about 0.5:1, about 0:1 to about 0.4:1, about 0:1 to about 0.3:1, about 0:1 to about 0.2:1, or >1.05;
e. the sum of (R²═SO₃R⁵+R³═SO₃R⁵+R⁴═SO₃R⁵) to R¹═SO₃R⁵ is about 0:1 to about 0.75:1, about 0:1 to about 0.7:1, about 0:1 to about 0.6:1, about 0:1 to about 0.55:1, about 0:1 to about 0.5:1, or >1.65:1
f. R²═SO₃R⁵ to R²═H is about 0:1 to about 0.7:1, about 0:1 to about 0.6:1, about 0:1 to about 0.55:1, about 0:1 to about 0.5:1, about 0:1 to about 0.4:1, about 0:1 to about 0.3:1, about 0:1 to about 0.2:1, about 0:1 to about 0.1:1, about 0:1 to about 0.05:1; and/or
g. R⁴═SO₃R⁵ to R⁴═H is about 0:1 to about 0.7:1, about 0:1 to about 0.5:1, about 0:1 to about 0.3:1, about 0:1 to about 0.25:1, about 0:1 to about 0.2:1, about 0:1 to about 0.15:1, about 0:1 to about 0.1:1, or about 0:1 to about 0.05:1.

In specific embodiments, the ratio of R⁴═SO₃R⁵ to R²═SO₃R⁵ is about 0:1 to about 0.7:1. In some specific embodiments, the ratio of R⁴═SO₃R⁵ to R²═SO₃R⁵ is about 0:1 to about 0.6:1. In some specific embodiments, the ratio of R⁴═SO₃R⁵ to R²═SO₃R⁵ is about 0:1 to about 0.55:1. In some specific embodiments, the ratio of R⁴═SO₃R⁵ to R²═SO₃R⁵ is about 0:1 to about 0.5:1. In some specific embodiments, the ratio of R⁴═SO₃R⁵ to R²═SO₃R⁵ is about 0:1 to about 0.4:1. In some specific embodiments, the ratio of R⁴═SO₃R⁵ to R²═SO₃R⁵ is about 0:1 to about 0.3:1. In some specific embodiments, the ratio of R⁴═SO₃R⁵ to R²═SO₃R⁵ is about 0:1 to about 0.2:1. In some specific embodiments, the ratio of R⁴═SO₃R⁵ to R²═SO₃R⁵ is >1.05. In some specific embodiments, the ratio of R⁴═SO₃R⁵ to R²═SO₃R⁵ is >1.1. In some specific embodiments, the ratio of R⁴═SO₃R⁵ to R²═SO₃R⁵ is >1.15. In some specific embodiments, the ratio of R⁴═SO₃R⁵ to R²═SO₃R⁵ is >1.2. In some specific embodiments, the ratio of R⁴═SO₃R⁵ to R²═SO₃R⁵ is >1.25. In some specific embodiments, the ratio of R⁴═SO₃R⁵ to R²═SO₃R⁵ is >1.3. In some specific embodiments, the ratio of R⁴═SO₃R⁵ to R²═SO₃R⁵ is >1.4. In some specific embodiments, the ratio of R⁴═SO₃R⁵ to R²═SO₃R⁵ is >1.5. In some specific embodiments, the ratio of R⁴═SO₃R⁵ to R²═SO₃R⁵ is >1.6. In some specific embodiments, the ratio of R⁴═SO₃R⁵ to R²═SO₃R⁵ is >1.7. In some specific embodiments, the ratio of R⁴═SO₃R⁵ to R²═SO₃R⁵ is >1.8. In some specific embodiments, the ratio of R⁴═SO₃R⁵ to R²═SO₃R⁵ is >1.9. In some specific embodiments, the ratio of R⁴═SO₃R⁵ to R²═SO₃R⁵ is >2.0. In some specific embodiments, the ratio of R⁴═SO₃R⁵ to R²═SO₃R⁵ is >3. In some specific embodiments, the ratio of R⁴═SO₃R⁵ to R²═SO₃R⁵ is >4. In some specific embodiments, the ratio of R⁴═SO₃R⁵ to R²═SO₃R⁵ is >5.

In some embodiments, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, or less than 3% of R¹ groups are SO₃R⁵. In certain embodiments, less than 10%, less than 9%, less than 8%, less than 7%, less than 6%, less than 5%, less than 4%, or less than 3% of R² groups are SO₃R⁵. In certain embodiments, less than 10%, less than 9%, less than 8%, less than 7%, less than 6%, less than 5%, less than 4%, or less than 3% of R⁴ groups are SO₃R⁵.

In certain embodiments, the compound is present in a human liver and less than 50%, less than 45%, less than 40%, less than 35%, less than 30%, less than 25%, less than 20%, or less than 15% of R¹ groups are SO₃R⁵. In certain embodiments, the compound is present in a human liver and less than 40%, less than 35%, less than 30%, less than 25%, less than 20%, or less than 15% of R² groups are SO₃R⁵. In certain embodiments, the compound is present in a human liver and less than 28%, less than 25%, less than 20%, less than 15%, less than 10%, or less than 5% of R⁴ groups are SO₃R⁵. In certain embodiments, the compound is present in a mammalian tissue or organ, wherein the tissue or organ is any one of those set forth in Table 1 and the compound is sulfated in one or more amount as set forth in Table 1.

In some embodiments, the compound comprises various epimers of X and/or Y. Specifically, in certain embodiments, each Y is independently selected from the C5 epimers glucuronic acid and iduronic acid. In some embodiments, n=5-250. In certain embodiments, n=10-200.

### Screening Processes

Provided in some embodiments is a process for identifying a compound that modulates heparan sulfate biosynthesis comprising:
a. contacting a cell with the compound in combination with a labeled probe that binds heparan sulfate;
b. incubating the cell, compound and labeled probe;
c. collecting the labeled probe that is bound to heparan sulfate; and
d. detecting or measuring the amount of labeled probe bound to heparan sulfate.

In more specific embodiments, provided herein is a process for identifying a compound that selectively modulates heparan sulfate biosynthesis comprising:
a. contacting a cell with the compound
b. contacting the cell and compound combination with a first labeled probe and a second labeled probe, wherein the first labeled probe binds heparan sulfate and the second labeled probe binds at least one glycan (e.g., a GAG, a sulfated GAG, an extracellular glycan, or the like) other than heparan sulfate;
c. incubating the cell, compound, the first labeled probe, and the second labeled probe;
d. collecting the first labeled probe that is bound to heparan sulfate;
e. collecting the second labeled probe that is bound to at least one glycan (e.g., a GAG, a sulfated GAG, an extracellular glycan, or the like) other than heparan sulfate;
f. detecting or measuring the amount of first labeled probe bound to heparan sulfate; and
g. detecting or measuring the amount of the second labeled probe bound to at least one glycan (e.g., a GAG, a sulfated GAG, an extracellular glycan, or the like) other than heparan sulfate.

Similarly, in some embodiments provided herein is a process for identifying compounds that selectively modulate heparan sulfate biosynthesis comprising:
a. contacting a first cell with the compound
b. contacting the first cell and compound combination with a first labeled probe, wherein the first labeled probe binds heparan sulfate;
c. incubating the first cell, compound, the first labeled probe, and the second labeled probe;
d. collecting the first labeled probe that is bound to heparan sulfate;
e. detecting or measuring the amount of first labeled probe bound to heparan sulfate;
f. contacting a second cell with the compound, wherein the second cell is of the same type as the first cell;
g. contacting the second cell and compound combination with a second labeled probe, wherein the second labeled probe binds at least one glycan (e.g., a GAG, a sulfated GAG, an extracellular glycan, or the like) other than heparan sulfate;
h. collecting the second labeled probe that is bound to at least one glycan (e.g., a GAG, a sulfated GAG, an extracellular glycan, or the like) other than heparan sulfate; and
i. detecting or measuring the amount of the second labeled probe bound to at least one glycan (e.g., a GAG, a sulfated GAG, an extracellular glycan, or the like) other than heparan sulfate.

In specific embodiments, the cell used in a process described herein is one that expresses heparan sulfate and, optionally, a second glycan that is not heparan sulfate. In certain embodiments, incubation of the cell, compound and labeled probe, transforms expressed glycan (e.g., heparan sulfate or a non-heparan sulfate glycan) and labeled probe into a probe-glycan complex. In some embodiments, collection of the probe-glycan complex involves purification with any suitable technique (e.g., chromotography, electrophoresis, capillary electrophoresis, gel electrophoresis, thin layer chromatography (TLC), high performance liquid chromatography (HPLC), ion exchange chromatography, reverse phase chromatography, gel permeation chromatography (GPC), gas chromatography (GC), precipitation, or the like). In some embodiments, detection and/or measurement of probe-glycan (e.g., heparan sulfate or a non-heparan sulfate glycan) complex is performed with an analytical instrument (e.g., spectrometer, mass spectrometer (MS), nuclear magnetic resonance (NMR) spectrometer, UV-Vis spectrometer, fluorimeter, or the like).

In some embodiments, the process further comprises comparing the amount of first labeled probe bound to heparan sulfate to the amount of the second labeled probe bound to at least one glycan other than heparan sulfate (e.g., to determine a ratio of the amount of first labeled probe bound to the amount of second labeled probe bound under substantially similar conditions).

In certain embodiments, a label utilized in any process described herein is any suitable label such as, by way of non-limiting example, a fluorescent label, a dye, a radiolabel, or the like. In some embodiments, the labeled probe comprises a biotinyl moiety and the process further comprises tagging the labeled probe with streptavidin-Cy5-PE. In certain embodiments, the first probe is any heparan binding lectin, e.g., a growth factor. In specific embodiments, the growth factor is, by way of non-limiting example, FGF (e.g., FGF2) or VEGF. In various embodiments, the amount of bound labeled probes are detected in any suitable manner, e.g., with a fluorimeter, a radiation detector, or the like.

In certain embodiments, the first and second probes are labeled in a manner so as to be independently detectable. In some embodiments, the first and second probes are contacted to the cells separately (i.e., to different cells of the same type) and independently analyzed. In some embodiments, the at least one glycan (e.g., a GAG, a sulfated GAG, an extracellular glycan, or the like) other than heparan sulfate is, by way of non-limiting example, chondroitin sulfate, dermatan sulfate, keratin, O-linked glycans, N-linked glycans, gangliosides, or the like. Furthermore, in some embodiments, a third labeled probe that binds at least one glycan (e.g., a GAG, a sulfated GAG, an extracellular glycan, or the like) not bound by the first or second labeled probe is also utilized. Additional labeled probes are also optionally utilized.

Second and additional labeled probes include any labeled compound or labeled lectin suitable (e.g., a labeled compound or lectin that binds a non-heparan sulfate GAG, a non-heparan sulfate glycan, a non-sulfated GAG, an extracellular glycan, an O-linked glycan, an N-linked glycan, a ganglioside, chondroitin sulfate, dermatan sulfate, keratin sulfate, and/or hyaluronan). In some embodiments, labeled probes included labeled forms of one or more of, by way of non-limiting example, Wheat Germ Agglutinin (WGA) from Triticum vulgaris (as a probe for binding N-linked and O-linked glycans with terminal GlcNAc residues and clustered sialic acid residues); Phaseolus Vulgaris Agglutinin (PHA) from Phaseolus vulgaris (as a probe for binding N-linked glycans); Cholera Toxin B-subunit (CTB) from Vibrio cholera (as a probe for binding sialic acid modified glycolipids); Concanavalin A (ConA) from Canavalia ensiformis (as a probe for binding mannose residues in N-linked glycans); and/or Jacalin from Artocarpus integrifolia (as a probe for binding O-linked glycans). In specific embodiments, labeled forms of each of Wheat Germ Agglutinin (WGA) from Triticum vulgaris (as a probe for binding N-linked and O-linked glycans with terminal GlcNAc residues and clustered sialic acid residues); Phaseolus Vulgaris Agglutinin (PHA) from Phaseolus vulgaris (as a probe for binding N-linked glycans); and Cholera Toxin B-subunit (CTB) from Vibrio cholera (as a probe for binding sialic acid modified glycolipids) are utilized.

Contact with first, second and additional labeled probes occurs in parallel, concurrently, or sequentially. In certain embodiments, contact the compounds and multiple probes allows identification of selective heparan sulfate inhibitors.

In some embodiments, the cell is a mammalian cell, an insect cell, an amphibian cell, or any other suitable cell that expresses and/or is engineered to express heparan sulfate and/or another glycan of interest (e.g., chondroitin sulfate, dermatan sulfate, keratin, O-linked glycans, N-linked glycans, gangliosides, or the like). In certain embodiments, the cell is a the mammalian cell (e.g., human cell) and is selected from any suitable mammalian cell. In specific embodiments, the mammalian cell is, by way of non-limiting example, a human cancer cell (e.g., human cervical cancer cell (HeLa)), a human ovarian cancer cell (SKOV), a human lung cancer cell (Hal8), a human medulloblastoma cancer cell (DAOY), a Chinese Hamster Ovary (CHO) cell, or a human primary cell. In certain embodiments, included herein are processes wherein the cell includes a plurality (e.g., 2, 3, 4 or all) of a human cancer cell (e.g., human cervical cancer cell (HeLa)), a human ovarian cancer cell (SKOV), a human lung cancer cell (Hal8), a human medulloblastoma cancer cell (DAOY), and/or a Chinese Hamster Ovary (CHO) cell. Contact with such cells optionally occurs in parallel, concurrently, or sequentially. In certain embodiments, contact of multiple cells identification of heparan sulfate inhibitors (e.g., selective heparan sulfate inhibitors) that inhibit heparan sulfate biosynthesis in multiple cell lines. In some instances, utilization of a plurality of cell lines allows the elimination or minimization of false positives in identifying heparan sulfate inhibitors.

Thus, in some embodiments, any process described herein comprises contacting the compound to a first cell (type), contacting the compound to a second cell (type), and, optionally, contacting the compound to additional cells (types), and repeating the process described for each of the first, second and any additional cell types utilized (e.g., to determine if a heparan sulfate inhibitor is selective for multiple cell lines or to determine which types of cell lines that the heparan sulfate inhibitor selectively targets). Furthermore, in such embodiments, the process further comprises comparing the amount of labeled probe (or the amount of first, second or any additional labeled probe) that is bound in each type of cell (e.g., to determine selectively of inhibiting heparan sulfate biosynthesis compared to the biosynthesis of other types of glycans).

In some embodiments, once a compound that modulates heparan sulfate biosynthesis is determined by the process described, a similar process is optionally utilized to determine whether or not the compound selectively modulates heparan sulfate biosynthesis. Specifically, selectivity of a compound that modulates heparan sulfate biosynthesis is determined by utilizing a similar process as described for determining whether or not the compound modulates heparan sulfate biosynthesis, e.g., by:
a. contacting a mammalian cell with the compound in combination with a labeled probe that binds one or more non-heparan sulfate glycan (e.g., another GAG or other class of glycan);
b. incubating the mammalian cell, compound and labeled probe;
c. collecting the labeled probe that is bound to non-heparan sulfate glycan (e.g., another GAG or other class of glycan); and
d. detecting or measuring the amount of labeled probe bound to non-heparan sulfate glycan (e.g., another GAG or other class of glycan).

In various embodiments, this process is repeated for any number of non-heparan sulfate glycans (e.g., another GAG or other class of glycan). In some embodiments, the non-heparan sulfate glycans are, by way of non-limiting example, chondroitin sulfate, O-linked glycans, N-linked glycans, gangliosides, or the like.

Furthermore, provided in some embodiments herein is a process for identifying a compound that modulates heparan sulfate biosynthesis or a process for identifying the effect of a compound on heparan sulfate biosynthesis comprising:
a. collecting heparan sulfate from a first cell of a selected type, wherein the heparan sulfate is sulfated oligosaccharide comprising glucosylamine groups, uronic acid groups, and glucuronic acid groups;
b. cleaving the heparan sulfate into a plurality of disaccharide component parts;
c. measuring:
   i. the amount of heparan sulfate disaccharides produced by the first cell,
   ii. the amount of N-sulfation of the glucosylamine groups, 6-OH sulfation of the glucosylamine groups, the 3-OH sulfation of the glucosylamine groups, the 2-OH sulfation of the uronic acid groups, or a combination thereof of the heparan sulfate,
   iii. the pattern of sulfation (domain organization); or
   iv. a combination thereof; and
d. contacting and incubating a second cell of the selected type with the compound;
e. collecting modified heparan sulfate from the second cell, wherein the modified heparan sulfate is sulfated oligosaccharide comprising glucosylamine groups, uronic acid groups, and glucuronic acid groups;
f. cleaving the modified heparan sulfate into a plurality of disaccharide component parts;
g. measuring:
   i. the amount of heparan sulfate disaccharides produced by the second cell,
   ii. the amount of N-sulfation of the glucosylamine groups, 6-OH sulfation of the glucosylamine groups, the 3-OH sulfation of the glucosylamine groups, the 2-OH sulfation of the uronic acid groups, or a combination thereof of the modified heparan sulfate,
   iii. the pattern of sulfation (domain organization); or
   iv. a combination thereof; and
h. comparing:
   i. the amounts of heparan sulfate disaccharides produced by the first and second cells,
   ii. the amounts of 6-OH sulfation of the glucosylamine groups, the 3-OH sulfation of the glucosylamine groups, the 2-OH sulfation of the uronic acid groups, pattern of sulfation, or a combination thereof of the heparan sulfate and the modified heparan sulfate, or
   iii. a combination thereof.

In some embodiments, the cell is a mammalian cell, an insect cell, an amphibian cell, or any other suitable cell that expresses and/or is engineered to express heparan sulfate and/or another glycan of interest (e.g., chondroitin sulfate, dermatan sulfate, keratin, O-linked glycans, N-linked glycans, gangliosides, or the like). In certain embodiments, the cell is a mammalian cell (e.g., human cell) and is selected from any suitable mammalian cell. In specific embodiments, the mammalian cell is, by way of non-limiting example, a human cancer cell (e.g., human cervical cancer cell (HeLa)) a human ovarian cancer cell (SKOV), a human lung cancer cell (Hal8), a human medulloblastoma cancer cell (DAOY) or a human primary cell. Furthermore, in some embodiments, the process is repeated utilizing one or more additional cell types. In certain embodiments, the results (e.g., of (c), (g), and/or (h)) from the one or more additional cell types (e.g., a second, third, fourth, fifth or the like cell types) are compared to each other and the results (e.g., of (c), (g), and/or (h)) from the first cell type.

In certain embodiments, the heparan sulfate and/or the modified heparan sulfate are cleaved in any suitable manner. In some embodiments, the heparan sulfate and/or the modified heparan sulfate are cleaved using a suitable enzyme such as heparin lyase I, heparin lyase II, or heparin lyase III from flavobacterium heparinum, or in any other suitable chemical manner.

In some embodiments, the amount of disaccharide units present in the cell and/or the characteristic of the sulfation in a cell are determined in any suitable manner. For example, in some embodiments, the amount of disaccharide present and/or the amount of N-sulfation of the glucosylamine groups, 6-OH sulfation of the glucosylamine groups, the 3-OH sulfation of the glucosylamine groups, the 2-OH sulfation of the uronic acid groups, or a combination thereof is determined utilizing a carbazole assay, high performance liquid chromatography (HPLC), capillary electrophoresis, gel electrophoresis, mass spectrum (MS) analysis, nuclear magnetic resonance (NMR) analysis, or the like.

Moreover, in certain embodiments, the process described is a process for identifying compounds that selectively modulate heparan sulfate biosynthesis. In such embodiments, the process also comprises collecting one or more non-heparan sulfate glycan (e.g., a sulfated glycan, such as chondroitin sulfate, O-linked glycans, N-linked glycans, gangliosides, or the like) from the cell, both without incubation with the compound and with incubation with the compound; cleaving each of such non-heparan sulfate glycans; measuring the character of each of such non-heparan sulfate glycan; and comparing the character of the non-heparan sulfate glycan that was not incubated with the character of the non-heparan sulfate glycan that was incubated. In certain embodiments, the character includes, by way of non-limiting example, the chain length of the non-heparan sulfate glycan, the amount of sulfation of the non-heparan sulfate glycan, the location of sulfation of the non-heparan sulfate glycan, the structure of the non-heparan sulfate glycan, the composition of the non-heparan sulfate glycan, or the like. The structure of glycosaminoglycans, N-linked glycans, O-linked glycans, and lipid linked glycans can be determined using any suitable method, including, by way of non-limiting example, monosaccharide compositional analysis, capillary electrophoresis, gel electrophoresis, gel filtration, high performance liquid chromatography (HPLC), thin layer chromatography (TLC), mass spectrum (MS) analysis, nuclear magnetic resonance (NMR) analysis, or the like.

### Combinations

In certain instances, it is appropriate to administer at least one therapeutic compound described herein (i.e., any heparan sulfate modulator, or in specific embodiments inhibitor, described herein) in combination with another therapeutic agent. By way of example only, if one of the side effects experienced by a patient upon receiving one of the heparan sulfate modulators, or in specific embodiments heparan sulfate inhibitors, described herein is nausea, then it is appropriate in certain instances to administer an anti-nausea agent in combination with the initial therapeutic agent. Or, by way of example only, the therapeutic effectiveness of one of the heparan sulfate modulators, or in specific embodiments heparan sulfate inhibitors, described herein is enhanced by administration of an adjuvant (i.e., by itself the adjuvant has minimal therapeutic benefit, but in combination with another therapeutic agent, the overall therapeutic benefit to the patient is enhanced). Or, by way of example only, the benefit experienced by a patient is increased by administering one of heparan sulfate modulators, or in specific embodiments heparan sulfate inhibitors, described herein with another therapeutic agent (which also includes a therapeutic regimen) that also has therapeutic benefit. In any case, regardless of the disease, disorder or condition being treated, the overall benefit experienced by the patient is in some embodiments additive of the two therapeutic agents or in other embodiments, the patient experiences a synergistic benefit.

In some embodiments, the particular choice of compounds depends upon the diagnosis of the attending physicians and their judgment of the condition of the patient and the appropriate treatment protocol. The compounds are optionally administered concurrently (e.g., simultaneously, essentially simultaneously or within the same treatment protocol) or sequentially, depending upon the nature of the disease, disorder, or condition, the condition of the patient, and the actual choice of compounds used. In certain instances, the determination of the order of administration, and the number of repetitions of administration of each therapeutic agent during a treatment protocol, is based on an evaluation of the disease being treated and the condition of the patient.

In some embodiments, therapeutically-effective dosages vary when the drugs are used in treatment combinations. Methods for experimentally determining therapeutically-effective dosages of drugs and other agents for use in combination treatment regimens are described in the literature. For example, the use of metronomic dosing, i.e., providing more frequent, lower doses in order to minimize toxic side effects, has been described extensively in the literature. Combination treatment further includes periodic treatments that start and stop at various times to assist with the clinical management of the patient.

In some embodiments of the combination therapies described herein, dosages of the co-administered compounds vary depending on the type of co-drug employed, on the specific drug employed, on the disease or condition being treated and so forth. In addition, when co-administered with one or more biologically active agents, the compound provided herein is optionally administered either simultaneously with the biologically active agent(s), or sequentially. In certain instances, if administered sequentially, the attending physician will decide on the appropriate sequence of therapeutic compound described herein in combination with the additional therapeutic agent.

The multiple therapeutic agents (at least one of which is a heparan sulfate modulator or inhibitor described herein) are optionally administered in any order or even simultaneously. If simultaneously, the multiple therapeutic agents are optionally provided in a single, unified form, or in multiple forms (by way of example only, either as a single pill or as two separate pills). In certain instances, one of the therapeutic agents is optionally given in multiple doses. In other instances, both are optionally given as multiple doses. If not simultaneous, the timing between the multiple doses is any suitable timing, e.g, from more than zero weeks to less than four weeks. In some embodiments, the additional therapeutic agent is utilized to achieve remission (partial or complete) of a cancer, whereupon the therapeutic agent described herein (e.g., any heparan sulfate modulator, or in specific embodiments inhibitor, of Formula III-VII, or of FIG. 2) is subsequently administered. In addition, the combination methods, compositions and formulations are not to be limited to the use of only two agents; the use of multiple therapeutic combinations are also envisioned (including two or more therapeutic compounds described herein).

In certain embodiments, a dosage regimen to treat, prevent, or ameliorate the condition(s) for which relief is sought, is modified in accordance with a variety of factors. These factors include the disorder from which the subject suffers, as well as the age, weight, sex, diet, and medical condition of the subject. Thus, in various embodiments, the dosage regimen actually employed varies and deviates from the dosage regimens set forth herein.

In some embodiments, the pharmaceutical agents which make up the combination therapy disclosed herein are provided in a combined dosage form or in separate dosage forms intended for substantially simultaneous administration. In certain embodiments, the pharmaceutical agents that make up the combination therapy are administered sequentially, with either therapeutic compound being administered by a regimen calling for two-step administration. In some embodiments, two-step administration regimen calls for sequential administration of the active agents or spaced-apart administration of the separate active agents. In certain embodiments, the time period between the multiple administration steps varies, by way of non-limiting example, from a few minutes to several hours, depending upon the properties of each pharmaceutical agent, such as potency, solubility, bioavailability, plasma half-life and kinetic profile of the pharmaceutical agent.

In addition, the heparan sulfate modulators, or in specific embodiments heparan sulfate inhibitors, described herein also are optionally used in combination with procedures that provide additional or synergistic benefit to the patient. By way of example only, patients are expected to find therapeutic and/or prophylactic benefit in the methods described herein, wherein pharmaceutical composition of a compound disclosed herein and/or combinations with other therapeutics are combined with genetic testing to determine whether that individual is a carrier of a gene or gene mutation that is known to be correlated with certain diseases or conditions.

In various embodiments, the heparan sulfate modulators, or in specific embodiments heparan sulfate inhibitors, described herein and combination therapies are administered before, during or after the occurrence of a disease or condition. Timing of administering the composition containing a heparan sulfate modulator, or in specific embodiments heparan sulfate inhibitor, is optionally varied to suit the needs of the individual treated. Thus, in certain embodiments, the heparan sulfate modulators, or in specific embodiments heparan sulfate inhibitors, are used as a prophylactic and are administered continuously to subjects with a propensity to develop conditions or diseases in order to prevent the occurrence of the disease or condition. In some embodiments, the compounds and compositions are administered to a subject during or as soon as possible after the onset of the symptoms. The administration of the heparan sulfate modulators, or in specific embodiments heparan sulfate inhibitors, are optionally initiated within the first 48 hours of the onset of the symptoms, within the first 6 hours of the onset of the symptoms, or within 3 hours of the onset of the symptoms. The initial administration is achieved by any route practical, such as, for example, an intravenous injection, a bolus injection, infusion over 5 minutes to about 5 hours, a pill, a capsule, transdermal patch, buccal delivery, and the like, or combination thereof. In some embodiments, the compound should be administered as soon as is practicable after the onset of a disease or condition is detected or suspected, and for a length of time necessary for the treatment of the disease, such as, for example, from about 1 month to about 3 months. The length of treatment is optionally varied for each subject based on known criteria. In exemplary embodiments, the compound or a formulation containing the compound is administered for at least 2 weeks, between about 1 month to about 5 years, or from about 1 month to about 3 years.

In certain embodiments, therapeutic agents are combined with or utilized in combination with one or more of the following therapeutic agents in any combination: immunosuppressants or anti-cancer therapies (e.g., radiation, surgery or anti-cancer agents).

In some embodiments, one or more of the anti-cancer agents are proapoptotic agents. Examples of anti-cancer agents include, by way of non-limiting example: gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, or PD184352, Taxol™, also referred to as “paclitaxel”, which is a well-known anti-cancer drug which acts by enhancing and stabilizing microtubule formation, and analogs of Taxol™, such as Taxotere™. Compounds that have the basic taxane skeleton as a common structure feature, have also been shown to have the ability to arrest cells in the G2-M phases due to stabilized microtubules and may be useful for treating cancer in combination with the compounds described herein.

Further examples of anti-cancer agents include inhibitors of mitogen-activated protein kinase signaling, e.g., U0126, PD98059, PD184352, PD0325901, ARRY-142886, SB239063, SP600125, BAY 43-9006, wortmannin, or LY294002; Syk inhibitors; mTOR inhibitors; and antibodies (e.g., rituxan).

Other anti-cancer agents include Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-1 a; interferon gamma-1 b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer agents include: 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Yet other anticancer agents that include alkylating agents, antimetabolites, natural products, or hormones, e.g., nitrogen mustards (e.g., mechlorethamine, cyclophosphamide, chlorambucil, etc.), alkyl sulfonates (e.g., busulfan), nitrosoureas (e.g., carmustine, lomustine, etc.), or triazenes (decarbazine, etc.). Examples of antimetabolites include but are not limited to folic acid analog (e.g., methotrexate), or pyrimidine analogs (e.g., Cytarabine), purine analogs (e.g., mercaptopurine, thioguanine, pentostatin).

Examples of natural products include but are not limited to vinca alkaloids (e.g., vinblastin, vincristine), epipodophyllotoxins (e.g., etoposide), antibiotics (e.g., daunorubicin, doxorubicin, bleomycin), enzymes (e.g., L-asparaginase), or biological response modifiers (e.g., interferon alpha).

Examples of alkylating agents include, but are not limited to, nitrogen mustards (e.g., mechlorethamine, cyclophosphamide, chlorambucil, melphalan, etc.), ethylenimine and methylmelamines (e.g., hexamethylmelamine, thiotepa), alkyl sulfonates (e.g., busulfan), nitrosoureas (e.g., carmustine, lomustine, semustine, streptozocin, etc.), or triazenes (decarbazine, etc.). Examples of antimetabolites include, but are not limited to folic acid analog (e.g., methotrexate), or pyrimidine analogs (e.g., fluorouracil, floxuridine, Cytarabine), purine analogs (e.g., mercaptopurine, thioguanine, pentostatin.

Examples of hormones and antagonists include, but are not limited to, adrenocorticosteroids (e.g., prednisone), progestins (e.g., hydroxyprogesterone caproate, megestrol acetate, medroxyprogesterone acetate), estrogens (e.g., diethylstilbestrol, ethinyl estradiol), antiestrogen (e.g., tamoxifen), androgens (e.g., testosterone propionate, fluoxymesterone), antiandrogen (e.g., flutamide), gonadotropin releasing hormone analog (e.g., leuprolide). Other agents that can be used in the methods and compositions described herein for the treatment or prevention of cancer include platinum coordination complexes (e.g., cisplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide).

In some embodiments, provided herein is a method of treating lymphoma comprising administering a therapeutically effective amount of a compound described herein in combination with an antibody to CD20 and/or a CHOP (cyclophosphamide, doxorubicin, vincristine, and prednisone) therapy. In certain embodiments, provided herein is a method of treating leukemia comprising administering a therapeutically effective amount of a compound described herein in combination with ATRA, methotrexate, cyclophosphamide and the like.

### Pharmaceutical Compositions

In certain embodiments, pharmaceutical compositions are formulated in a conventional manner using one or more physiologically acceptable carriers including, e.g., excipients and auxiliaries which facilitate processing of the active compounds into preparations which are suitable for pharmaceutical use. In certain embodiments, proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

A pharmaceutical composition, as used herein, refers to a mixture of a heparan sulfate modulator, or in specific embodiments heparan sulfate inhibitor (e.g., a selective heparan sulfate modulator, or in specific embodiments inhibitor) described herein, such as, for example, a compound of Formula III-VII, or FIG. 2, with other chemical components, such as carriers, stabilizers, diluents, dispersing agents, suspending agents, thickening agents, and/or excipients. In certain instances, the pharmaceutical composition facilitates administration of the heparan sulfate modulator, or in specific embodiments heparan sulfate inhibitor (e.g., selective heparan sulfate modulator or inhibitor) to an individual or cell. In certain embodiments of practicing the methods of treatment or use provided herein, therapeutically effective amounts of heparan sulfate modulators, or in specific embodiments heparan sulfate inhibitors (e.g., selective heparan sulfate modulators or inhibitors) described herein are administered in a pharmaceutical composition to an individual having a disease, disorder, or condition to be treated. In specific embodiments, the individual is a human. As discussed herein, the heparan sulfate modulators, or in specific embodiments heparan sulfate inhibitors, described herein are either utilized singly or in combination with one or more additional therapeutic agents.

In certain embodiments, the pharmaceutical formulations described herein are administered to an individual in any manner, including one or more of multiple administration routes, such as, by way of non-limiting example, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a compound described herein are optionally manufactured in a conventional manner, such as, by way of example only, by means of conventional mixing, dissolving, granulating, dragee-making, levigating, emulsifying, encapsulating, entrapping or compression processes.

In certain embodiments, a pharmaceutical compositions described herein includes one or more heparan sulfate modulator, or in specific embodiments heparan sulfate inhibitor, described herein, e.g., a compound of Formula III-VII or FIG. 2, as an active ingredient in free-acid or free-base form, or in a pharmaceutically acceptable salt form. In some embodiments, the compounds described herein are utilized as an N-oxide or in a crystalline or amorphous form (i.e., a polymorph). In certain embodiments, an active metabolite or prodrug of a compound described herein is utilized. In some situations, a compound described herein exists as tautomers. All tautomers are included within the scope of the compounds presented herein. In certain embodiments, a compound described herein exists in an unsolvated or solvated form, wherein solvated forms comprise any pharmaceutically acceptable solvent, e.g., water, ethanol, and the like. The solvated forms of the heparan sulfate modulators, or in specific embodiments heparan sulfate inhibitors, presented herein are also considered to be disclosed herein.

A “carrier” includes, in some embodiments, a pharmaceutically acceptable excipient and is selected on the basis of compatibility with heparan sulfate inhibitors disclosed herein, such as, compounds of Formula III-VII or FIG. 2, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Moreover, in certain embodiments, the pharmaceutical compositions described herein is formulated as a dosage form. As such, in some embodiments, provided herein is a dosage form comprising a heparan sulfate modulator or inhibitor described herein, e.g., a compound of Formula III-VII or FIG. 2, suitable for administration to an individual. In certain embodiments, suitable dosage forms include, by way of non-limiting example, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

The pharmaceutical solid dosage forms described herein optionally include an additional therapeutic compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In some aspects, using standard coating procedures, such as those described in Remington's Pharmaceutical Sciences, 20th Edition (2000), a film coating is provided around the formulation of the heparan sulfate modulator or inhibitor of Formula III-VII or FIG. 2. In one embodiment, a heparan sulfate modulator or inhibitor described herein is in the form of a particle and some or all of the particles of the compound are coated. In certain embodiments, some or all of the particles of a heparan sulfate modulator or inhibitor described herein are microencapsulated. In some embodiment, the particles of the heparan sulfate modulator or inhibitor described herein are not microencapsulated and are uncoated.

In certain embodiments, the pharmaceutical composition described herein is in unit dosage forms suitable for single administration of precise dosages. In unit dosage form, the formulation is divided into unit doses containing appropriate quantities of one or more therapeutic compound. In some embodiments, the unit dosage is in the form of a package containing discrete quantities of the formulation. Non-limiting examples are packaged tablets or capsules, and powders in vials or ampoules. Aqueous suspension compositions are optionally packaged in single-dose non-reclosable containers. In some embodiments, multiple-dose re-closeable containers are used. In certain instances, multiple dose containers comprise a preservative in the composition. By way of example only, formulations for parenteral injection are presented in unit dosage form, which include, but are not limited to ampoules, or in multi-dose containers, with an added preservative.

### EXAMPLES

### Example 1

### 4-(4-(3,4-dimethoxyphenyl)-6-phenylpyrimidin-2-yl)morpholine

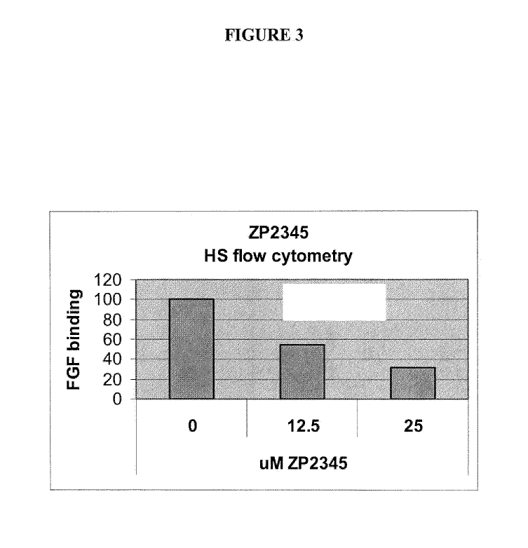

FIG. 2 illustrates the impact of 4-(4-(3,4-dimethoxyphenyl)-6-phenylpyrimidin-2-yl)morpholine on the ability of FGF2 to bind to heparan sulfate in human cervical cancer cells (HeLa). FGF2 binding to heparan sulfate is measured by culturing HeLa cells in the presence of the heparan sulfate modulator. After 2 days of growth, the cells are released with 5 mM EDTA and probed with biotinylated FGF2 for 30 minutes on ice. After washing unbound FGF2, bound FGF2 is detected with streptavidin-Cy5-PE. After washing to remove unbound streptavidin-Cy5-PE, the bound probe is quantified using a flow cytometry. The heparan sulfate inhibitors are tested on at least three independent occasions, in duplicate over a dose range. Heparan sulfate specificity is determined by probing with lectins to other glycan classes (chondroitin sulfate, O-linked, N-linked, etc.). FIG. 3 illustrates the impact of 4-(4-(3,4-dimethoxyphenyl)-6-phenylpyrimidin-2-yl)morpholine on the sulfation of heparan sulfate found in human cervical cancer cells (HeLa). The sulfation of heparan sulfate is determined by culturing HeLa cells in the presence of the heparan sulfate modulator. Heparan sulfate is isolated from treated and untreated cells by lysing the cells in 0.1 N NaOH. After neutralization, the cell lysate is treated with pronase to degrade the core proteins. Subsequently, the heparan sulfate is purified on diethylaminoethyl (DEAE) resin, eluted with 1.0 M NaCl, desalted using gel filtration and dried. To characterize the composition of heparan sulfate produced in the presence of the heparan sulfate modulator, the isolate heparan sulfate is digested to disaccharides using a blend of heparin lyase I, II, and III (from flavobacterium heparinum). The disaccharides are quantified by HPLC using a propac PA1 column using known heparan sulfate disaccharides as standards. Specifically, FIG. 3A illustrates the impact of 4-(4-(3,4-dimethoxyphenyl)-6-phenylpyrimidin-2-yl)morpholine on the amount of specific disaccharide units; and FIG. 3B illustrates the impact of 4-(4-(3,4-dimethoxyphenyl)-6-phenylpyrimidin-2-yl)morpholine on the amount of specific glucosamine and uronic acid modifications.

### Example 2

### 7-((3-chlorophenyl)(pyridin-2-ylamino)methyl)-2-methylquinolin-8-ol

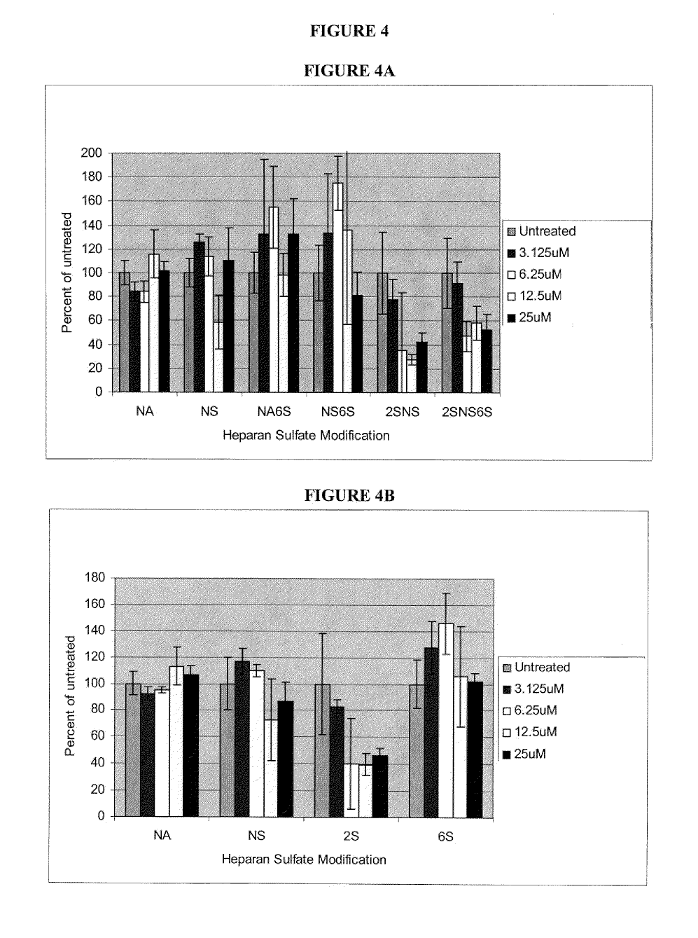

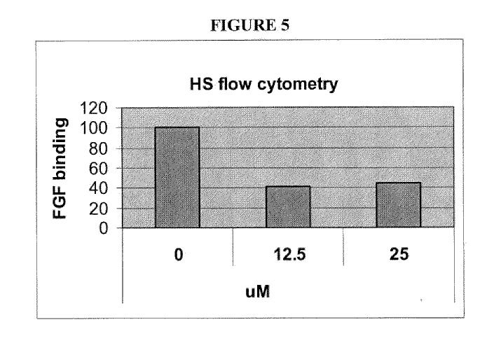

FIG. 4 illustrates the impact of 7-((3-chlorophenyl)(pyridin-2-ylamino)methyl)-2-methylquinolin-8-ol on the ability of FGF2 to bind to heparan sulfate in human cervical cancer cells (HeLa). FGF2 binding to heparan sulfate is measured by culturing HeLa cells in the presence of the heparan sulfate modulator. After 2 days of growth, the cells are released with 5 mM EDTA and probed with biotinylated FGF2 for 30 minutes on ice. After washing unbound FGF2, bound FGF2 is detected with streptavidin-Cy5-PE. After washing to remove unbound streptavidin-Cy5-PE, the bound probe is quantified using a flow cytometry. The heparan sulfate inhibitors are tested on at least three independent occasions, in duplicate over a dose range. Heparan sulfate specificity is determined by probing with lectins to other glycan classes (chondroitin sulfate, O-linked, N-linked, etc.). FIG. 5 illustrates the impact of 7-((3-chlorophenyl)(pyridin-2-ylamino)methyl)-2-methylquinolin-8-ol on the sulfation of heparan sulfate found in human cervical cancer cells (HeLa). The sulfation of heparan sulfate is determined by culturing HeLa cells in the presence of the heparan sulfate modulator. Heparan sulfate is isolated from treated and untreated cells by lysing the cells in 0.1 N NaOH. After neutralization, the cell lysate is treated with pronase to degrade the core proteins. Subsequently, the heparan sulfate is purified on diethylaminoethyl (DEAE) resin, eluted with 1.0 M NaCl, desalted using gel filtration and dried. To characterize the composition of heparan sulfate produced in the presence of the heparan sulfate modulator, the isolate heparan sulfate is digested to disaccharides using a blend of heparin lyase I, II, and III (from flavobacterium heparinum). The disaccharides are quantified by HPLC using a propac PA1 column using known heparan sulfate disaccharides as standards. Specifically, FIG. 5A illustrates the impact of 7-((3-chlorophenyl)(pyridin-2-ylamino)methyl)-2-methylquinolin-8-ol on the amount of specific disaccharide units; and FIG. 5B illustrates the impact of 7-((3-chlorophenyl)(pyridin-2-ylamino)methyl)-2-methylquinolin-8-ol on the amount of specific glucosamine and uronic acid modifications.

### Example 3

### 7-((2-fluorophenyl)(pyridin-2-ylamino)methyl)-2-methylquinolin-8-ol

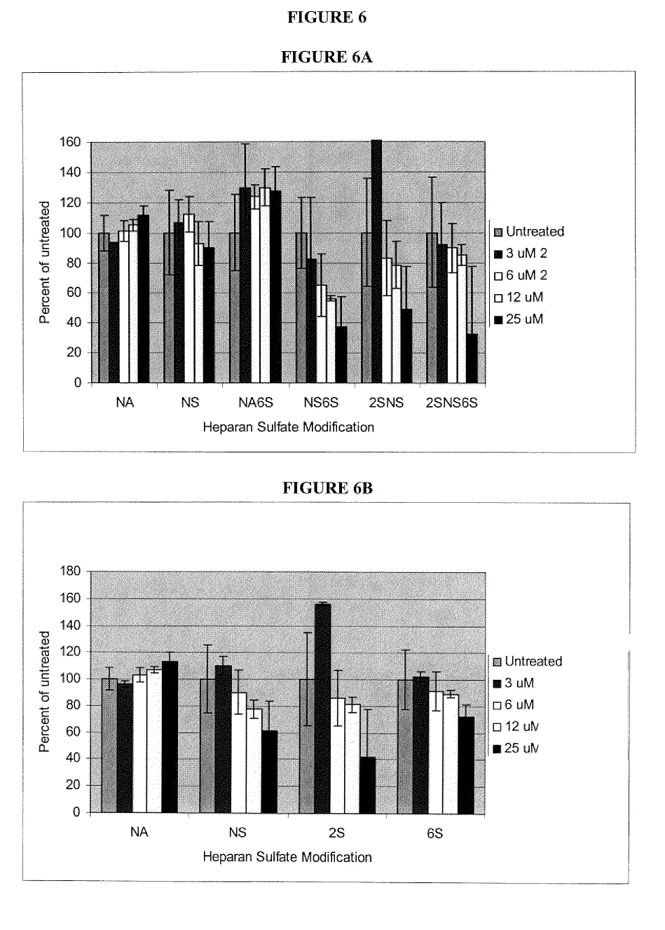

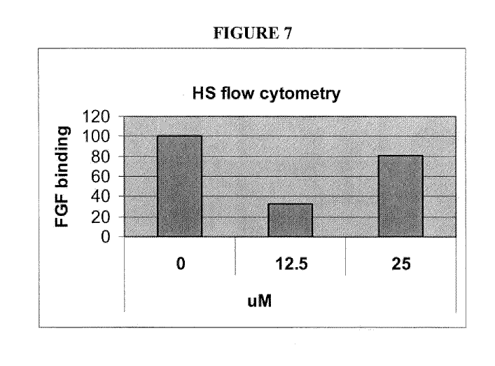

FIG. 6 illustrates the impact of 7-((2-fluorophenyl)(pyridin-2-ylamino)methyl)-2-methylquinolin-8-ol on the ability of FGF2 to bind to heparan sulfate in human cervical cancer cells (HeLa). FGF2 binding to heparan sulfate is measured by culturing HeLa cells in the presence of the heparan sulfate modulator. After 2 days of growth, the cells are released with 5 mM EDTA and probed with biotinylated FGF2 for 30 minutes on ice. After washing unbound FGF2, bound FGF2 is detected with streptavidin-Cy5-PE. After washing to remove unbound streptavidin-Cy5-PE, the bound probe is quantified using a flow cytometry. The heparan sulfate inhibitors are tested on at least three independent occasions, in duplicate over a dose range. Heparan sulfate specificity is determined by probing with lectins to other glycan classes (chondroitin sulfate, O-linked, N-linked, etc.). FIG. 7 illustrates the impact of 7-((2-fluorophenyl)(pyridin-2-ylamino)methyl)-2-methylquinolin-8-ol on the sulfation of heparan sulfate found in human cervical cancer cells (HeLa). The sulfation of heparan sulfate is determined by culturing HeLa cells in the presence of the heparan sulfate modulator. Heparan sulfate is isolated from treated and untreated cells by lysing the cells in 0.1 N NaOH. After neutralization, the cell lysate is treated with pronase to degrade the core proteins. Subsequently, the heparan sulfate is purified on diethylaminoethyl (DEAE) resin, eluted with 1.0 M NaCl, desalted using gel filtration and dried. To characterize the composition of heparan sulfate produced in the presence of the heparan sulfate modulator, the isolate heparan sulfate is digested to disaccharides using a blend of heparin lyase I, II, and III (from flavobacterium heparinum). The disaccharides are quantified by HPLC using a propac PA1 column using known heparan sulfate disaccharides as standards. Specifically, FIG. 7A illustrates the impact of 7-((2-fluorophenyl)(pyridin-2-ylamino)methyl)-2-methylquinolin-8-ol on the amount of specific disaccharide units; and FIG. 7B illustrates the impact of 7-((2-fluorophenyl)(pyridin-2-ylamino)methyl)-2-methylquinolin-8-ol on the amount of specific glucosamine and uronic acid modifications.

### Example 4

### 7-((4-chlorophenyl)(acetylamino)methyl)-5-nitroquinolin-8-ol

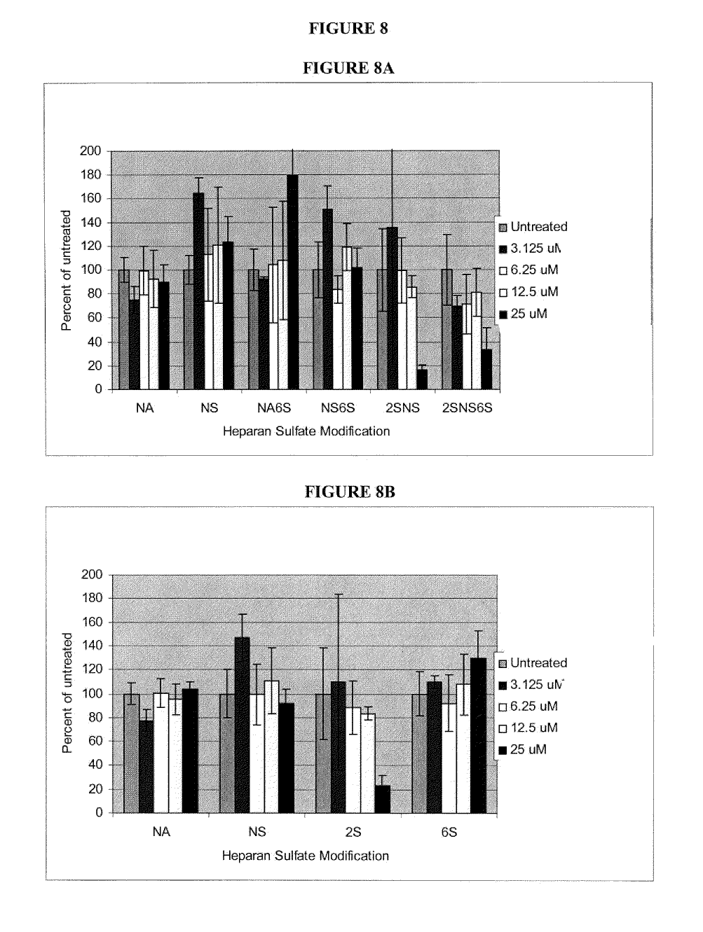

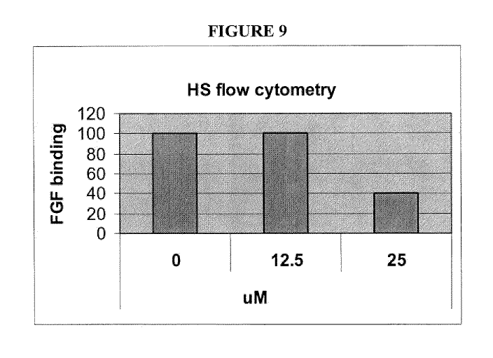

FIG. 8 illustrates the impact of 7-((3-chlorophenyl)(acetylamino)methyl)-5-nitroquinolin-8-ol on the ability of FGF2 to bind to heparan sulfate in human cervical cancer cells (HeLa). FGF2 binding to heparan sulfate is measured by culturing HeLa cells in the presence of the heparan sulfate modulator. After 2 days of growth, the cells are released with 5 mM EDTA and probed with biotinylated FGF2 for 30 minutes on ice. After washing unbound FGF2, bound FGF2 is detected with streptavidin-Cy5-PE. After washing to remove unbound streptavidin-Cy5-PE, the bound probe is quantified using a flow cytometry. The heparan sulfate inhibitors are tested on at least three independent occasions, in duplicate over a dose range. Heparan sulfate specificity is determined by probing with lectins to other glycan classes (chondroitin sulfate, O-linked, N-linked, etc.). FIG. 9 illustrates the impact of 7-((3-chlorophenyl)(acetylamino)methyl)-5-nitroquinolin-8-ol on the sulfation of heparan sulfate found in human cervical cancer cells (HeLa). The sulfation of heparan sulfate is determined by culturing HeLa cells in the presence of the heparan sulfate modulator. Heparan sulfate is isolated from treated and untreated cells by lysing the cells in 0.1 N NaOH. After neutralization, the cell lysate is treated with pronase to degrade the core proteins. Subsequently, the heparan sulfate is purified on diethylaminoethyl (DEAE) resin, eluted with 1.0 M NaCl, desalted using gel filtration and dried. To characterize the composition of heparan sulfate produced in the presence of the heparan sulfate modulator, the isolate heparan sulfate is digested to disaccharides using a blend of heparin lyase I, II, and III (from flavobacterium heparinum). The disaccharides are quantified by HPLC using a propac PA1 column using known heparan sulfate disaccharides as standards. Specifically, FIG. 9A illustrates the impact of 7-((3-chlorophenyl)(acetylamino)methyl)-5-nitroquinolin-8-ol on the amount of specific disaccharide units; and FIG. 9B illustrates the impact of 7-((3-chlorophenyl)(acetylamino)methyl)-5-nitroquinolin-8-ol on the amount of specific glucosamine and uronic acid modifications.

### Example 5

### 7-((thiophen-2-yl)(isobutoylamino)methyl)-5-nitroquinolin-8-ol

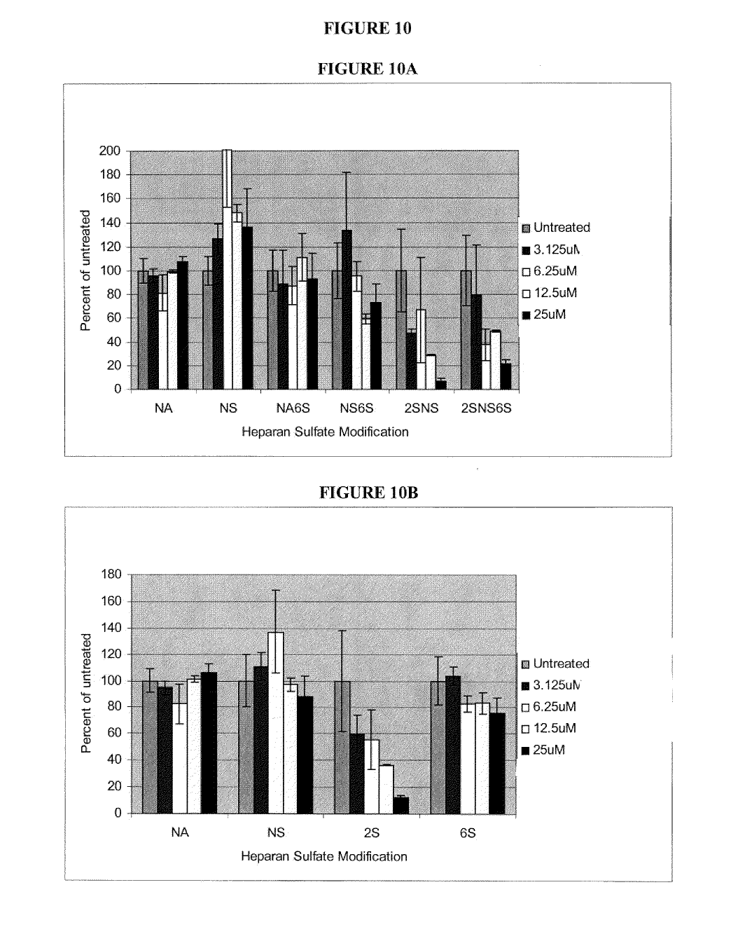

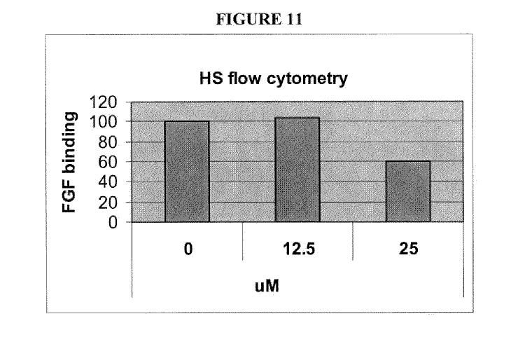

FIG. 10 illustrates the impact of 7-((thiophen-2-yl)(isobutoylamino)methyl)-5-nitroquinolin-8-ol on the ability of FGF2 to bind to heparan sulfate in human cervical cancer cells (HeLa). FGF2 binding to heparan sulfate is measured by culturing HeLa cells in the presence of the heparan sulfate modulator. After 2 days of growth, the cells are released with 5 mM EDTA and probed with biotinylated FGF2 for 30 minutes on ice. After washing unbound FGF2, bound FGF2 is detected with streptavidin-Cy5-PE. After washing to remove unbound streptavidin-Cy5-PE, the bound probe is quantified using a flow cytometry. The heparan sulfate inhibitors are tested on at least three independent occasions, in duplicate over a dose range. Heparan sulfate specificity is determined by probing with lectins to other glycan classes (chondroitin sulfate, O-linked, N-linked, etc.). FIG. 11 illustrates the impact of 7-((thiophen-2-yl)(isobutoylamino)methyl)-5-nitroquinolin-8-ol on the sulfation of heparan sulfate found in human cervical cancer cells (HeLa). The sulfation of heparan sulfate is determined by culturing HeLa cells in the presence of the heparan sulfate modulator. Heparan sulfate is isolated from treated and untreated cells by lysing the cells in 0.1 N NaOH. After neutralization, the cell lysate is treated with pronase to degrade the core proteins. Subsequently, the heparan sulfate is purified on diethylaminoethyl (DEAE) resin, eluted with 1.0 M NaCl, desalted using gel filtration and dried. To characterize the composition of heparan sulfate produced in the presence of the heparan sulfate modulator, the isolate heparan sulfate is digested to disaccharides using a blend of heparin lyase I, II, and III (from flavobacterium heparinum). The disaccharides are quantified by HPLC using a propac PA1 column using known heparan sulfate disaccharides as standards. Specifically, FIG. 11A illustrates the impact of 7-((thiophen-2-yl)(isobutoylamino)methyl)-5-nitroquinolin-8-ol on the amount of specific disaccharide units; and FIG. 11B illustrates the impact of 7-((thiophen-2-yl)(isobutoylamino)methyl)-5-nitroquinolin-8-ol on the amount of specific glucosamine and uronic acid modifications.

### Example 6

### 4-ethyl-7-(4-nitro-2-(trifluoromethyl)phenoxy)-2H-chromen-2-one

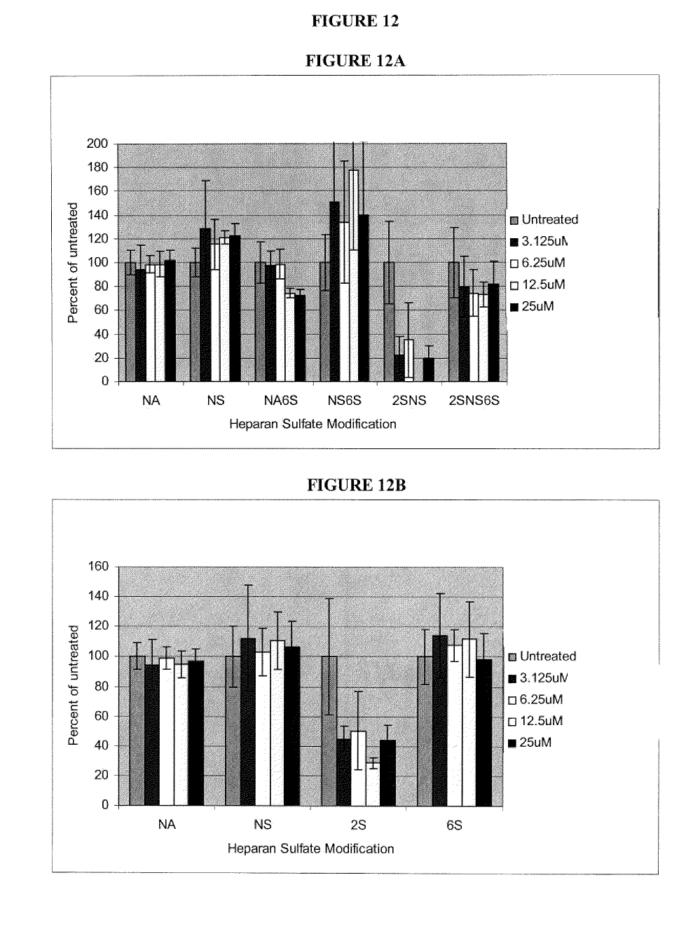

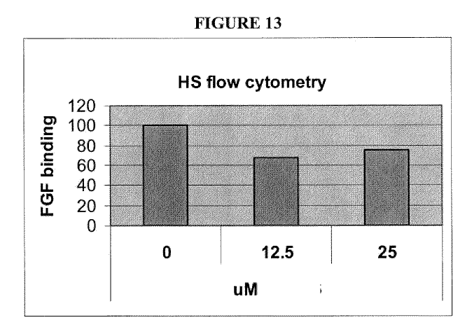

FIG. 12 illustrates the impact of 4-ethyl-7-(4-nitro-2-(trifluoromethyl)phenoxy)-2H-chromen-2-one on the ability of FGF2 to bind to heparan sulfate in human cervical cancer cells (HeLa). FGF2 binding to heparan sulfate is measured by culturing HeLa cells in the presence of the heparan sulfate modulator. After 2 days of growth, the cells are released with 5 mM EDTA and probed with biotinylated FGF2 for 30 minutes on ice. After washing unbound FGF2, bound FGF2 is detected with streptavidin-Cy5-PE. After washing to remove unbound streptavidin-Cy5-PE, the bound probe is quantified using a flow cytometry. The heparan sulfate inhibitors are tested on at least three independent occasions, in duplicate over a dose range. Heparan sulfate specificity is determined by probing with lectins to other glycan classes (chondroitin sulfate, O-linked, N-linked, etc.). FIG. 13 illustrates the impact of 4-ethyl-7-(4-nitro-2-(trifluoromethyl)phenoxy)-2H-chromen-2-one on the sulfation of heparan sulfate found in human cervical cancer cells (HeLa). The sulfation of heparan sulfate is determined by culturing HeLa cells in the presence of the heparan sulfate modulator. Heparan sulfate is isolated from treated and untreated cells by lysing the cells in 0.1 N NaOH. After neutralization, the cell lysate is treated with pronase to degrade the core proteins. Subsequently, the heparan sulfate is purified on diethylaminoethyl (DEAE) resin, eluted with 1.0 M NaCl, desalted using gel filtration and dried. To characterize the composition of heparan sulfate produced in the presence of the heparan sulfate modulator, the isolate heparan sulfate is digested to disaccharides using a blend of heparin lyase I, II, and III (from flavobacterium heparinum). The disaccharides are quantified by HPLC using a propac PA1 column using known heparan sulfate disaccharides as standards. Specifically, FIG. 13A illustrates the impact of 4-ethyl-7-(4-nitro-2-(trifluoromethyl)phenoxy)-2H-chromen-2-one on the amount of specific disaccharide units; and FIG. 13B illustrates the impact of 4-ethyl-7-(4-nitro-2-(trifluoromethyl)phenoxy)-2H-chromen-2-one on the amount of specific glucosamine and uronic acid modifications.

### Example 7

### Treating MPS

MPS IIIA mice (see Bhaumik M, Muller V J, Rozaklis T, Johnson L, Dobrenis K, Bhattacharyya R, Wurzelmann S, Finamore P, Hopwood J J, Walkley S U, Stanley P: A mouse model for mucopolysaccharidosis type III A (Sanfilippo syndrome); Glycobiol 1999, 9:1389-1396) are injected with an effective amount (e.g., about 100 mg/kg/day) of 4-(4-(3,4-dimethoxyphenyl)-6-phenylpyrimidin-2-yl)morpholine. Samples of mouse urine are incubated for one hour at 37° C. with two volumes of 0.1% cetylpyridinium chloride in 0.054 M Na₃ citrate (pH 4.8). Samples are centrifuged for 10 minutes at 3000 rpm and pellets are resuspended in 150 μL 2 M LiCl. Following addition of 800 μL absolute ethanol, samples are incubated at −20° C. for one hour and then centrifuged for 10 minutes at 3000 rpm. Pellets are resuspended in 200 μL of water, lyophilised, and then resuspended in 20 μL water. Purified glycosaminoglycan samples (0.2 μmol creatinine equivalents) are analysed on 40-50% linear gradient polyacrylamide gels. Samples are compared to results for a control MPS IIIA mouse (or population thereof) that was not exposed to 4-(4-(3,4-dimethoxyphenyl)-6-phenylpyrimidin-2-yl)morpholine.

### Example 8

### Treating MPS

MPS IIIA mice (see Bhaumik M, Muller V J, Rozaklis T, Johnson L, Dobrenis K, Bhattacharyya R, Wurzelmann S, Finamore P, Hopwood J J, Walkley S U, Stanley P: A mouse model for mucopolysaccharidosis type III A (Sanfilippo syndrome); Glycobiol 1999, 9:1389-1396) are injected with an effective amount (e.g., about 100 mg/kg/day) of 7-((3-chlorophenyl)(pyridin-2-ylamino)methyl)-2-methylquinolin-8-ol. Samples of mouse urine are incubated for one hour at 37° C. with two volumes of 0.1% cetylpyridinium chloride in 0.054 M Na₃ citrate (pH 4.8). Samples are centrifuged for 10 minutes at 3000 rpm and pellets are resuspended in 150 μL 2 M LiCl. Following addition of 800 μL absolute ethanol, samples are incubated at −20° C. for one hour and then centrifuged for 10 minutes at 3000 rpm. Pellets are resuspended in 200 μL of water, lyophilised, and then resuspended in 20 μL water. Purified glycosaminoglycan samples (0.2 μmol creatinine equivalents) are analysed on 40-50% linear gradient polyacrylamide gels. Samples are compared to results for a control MPS IIIA mouse (or population thereof) that was not exposed to 7-((3-chlorophenyl)(pyridin-2-ylamino)methyl)-2-methylquinolin-8-ol.

### Example 9

### Method of Treatment

Human Clinical Trial of the Safety and/or Efficacy of 4-(4-(3,4-dimethoxyphenyl)-6-phenylpyrimidin-2-yl)morpholine (or a pharmaceutically acceptable salt thereof) therapy

Objective: To determine the safety and pharmacokinetics of administered 4-(4-(3,4-dimethoxyphenyl)-6-phenylpyrimidin-2-yl)morpholine.

Study Design: This will be a Phase I, single-center, open-label, randomized dose escalation study followed by a Phase II study in cancer patients with a cancer that can be biopsied (e.g., pancreatic cancer, colorectal cancer, lung cancer, or ovarian cancer). Patients should not have had exposure to 4-(4-(3,4-dimethoxyphenyl)-6-phenylpyrimidin-2-yl)morpholine prior to the study entry. Patients must not have received treatment for their cancer within 2 weeks of beginning the trial. Treatments include the use of chemotherapy, hematopoietic growth factors, and biologic therapy such as monoclonal antibodies. The exception is the use of hydroxyurea for patients with WBC >30×103/μL. This duration of time appears adequate for wash out due to the relatively short-acting nature of most anti-leukemia agents. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I: Patients receive intravenous 4-(4-(3,4-dimethoxyphenyl)-6-phenylpyrimidin-2-yl)morpholine daily for 5 consecutive days or 7 days a week. Doses of 4-(4-(3,4-dimethoxyphenyl)-6-phenylpyrimidin-2-yl)morpholine may be held or modified for toxicity based on assessments as outlined below. Treatment repeats every 28 days in the absence of unacceptable toxicity. Cohorts of 3-6 patients receive escalating doses of 4-(4-(3,4-dimethoxyphenyl)-6-phenylpyrimidin-2-yl)morpholine until the maximum tolerated dose (MTD) for the 4-(4-(3,4-dimethoxyphenyl)-6-phenylpyrimidin-2-yl)morpholine is determined. The MTD is defined as the dose preceding that at which 2 of 3 or 2 of 6 patients experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II: Patients receive 4-(4-(3,4-dimethoxyphenyl)-6-phenylpyrimidin-2-yl)morpholine as in phase I at the MTD determined in phase I. Treatment repeats every 6 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they meet original eligibility criteria.

Blood Sampling Serial blood is drawn by direct vein puncture before and after administration of 4-(4-(3,4-dimethoxyphenyl)-6-phenylpyrimidin-2-yl)morpholine. Venous blood samples (5 mL) for determination of serum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics: Patients undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response: Patient response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon the cancer type and feasibility/availability, and the same imaging modality is utilized for similar cancer types as well as throughout each patient's study course. Response rates are determined using the RECIST criteria. (Therasse et al, J. Natl. Cancer Inst. 2000 Feb. 2; 92(3):205-16; http://ctep.cancer.gov/forms/TherasseRECISTJNCI.pdf). Patients also undergo cancer/tumor biopsy to assess changes in progenitor cancer cell phenotype and clonogenic growth by flow cytometry, Western blotting, and IHC, and for changes in cytogenetics by FISH or TaqMan PCR for specific chromosomal translocations. After completion of study treatment, patients are followed periodically for 4 weeks.

### Example 10

### Method of Treatment

Human Clinical Trial of the Safety and/or Efficacy of 7-((3-chlorophenyl)(pyridin-2-ylamino)methyl)-2-methylquinolin-8-ol (or a pharmaceutically acceptable salt thereof) therapy

Objective: To determine the safety and pharmacokinetics of administered 7-((3-chlorophenyl)(pyridin-2-ylamino)methyl)-2-methylquinolin-8-ol.

Study Design: This will be a Phase I, single-center, open-label, randomized dose escalation study followed by a Phase II study in cancer patients with a cancer that can be biopsied (e.g., pancreatic cancer, colorectal cancer, lung cancer, or ovarian cancer). Patients should not have had exposure to 7-((3-chlorophenyl)(pyridin-2-ylamino)methyl)-2-methylquinolin-8-ol prior to the study entry. Patients must not have received treatment for their cancer within 2 weeks of beginning the trial. Treatments include the use of chemotherapy, hematopoietic growth factors, and biologic therapy such as monoclonal antibodies. The exception is the use of hydroxyurea for patients with WBC >30×103/μL. This duration of time appears adequate for wash out due to the relatively short-acting nature of most anti-leukemia agents. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I: Patients receive intravenous 7-((3-chlorophenyl)(pyridin-2-ylamino)methyl)-2-methylquinolin-8-ol daily for 5 consecutive days or 7 days a week. Doses of 7-((3-chlorophenyl)(pyridin-2-ylamino)methyl)-2-methylquinolin-8-ol may be held or modified for toxicity based on assessments as outlined below. Treatment repeats every 28 days in the absence of unacceptable toxicity. Cohorts of 3-6 patients receive escalating doses of 7-((3-chlorophenyl)(pyridin-2-ylamino)methyl)-2-methylquinolin-8-ol until the maximum tolerated dose (MTD) for the 7-((3-chlorophenyl)(pyridin-2-ylamino)methyl)-2-methylquinolin-8-ol is determined. The MTD is defined as the dose preceding that at which 2 of 3 or 2 of 6 patients experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II: Patients receive 7-((3-chlorophenyl)(pyridin-2-ylamino)methyl)-2-methylquinolin-8-ol as in phase I at the MTD determined in phase I. Treatment repeats every 6 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they meet original eligibility criteria.

Blood Sampling Serial blood is drawn by direct vein puncture before and after administration of 7-((3-chlorophenyl)(pyridin-2-ylamino)methyl)-2-methylquinolin-8-ol. Venous blood samples (5 mL) for determination of serum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics: Patients undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response: Patient response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon the cancer type and feasibility/availability, and the same imaging modality is utilized for similar cancer types as well as throughout each patient's study course. Response rates are determined using the RECIST criteria. (Therasse et al, J. Natl. Cancer Inst. 2000 Feb. 2; 92(3):205-16; http://ctep.cancer.gov/forms/TherasseRECISTJNCI.pdf). Patients also undergo cancer/tumor biopsy to assess changes in progenitor cancer cell phenotype and clonogenic growth by flow cytometry, Western blotting, and IHC, and for changes in cytogenetics by FISH or TaqMan PCR for specific chromosomal translocations. After completion of study treatment, patients are followed periodically for 4 weeks.

### Example 11

### Substrate Reduction Therapy

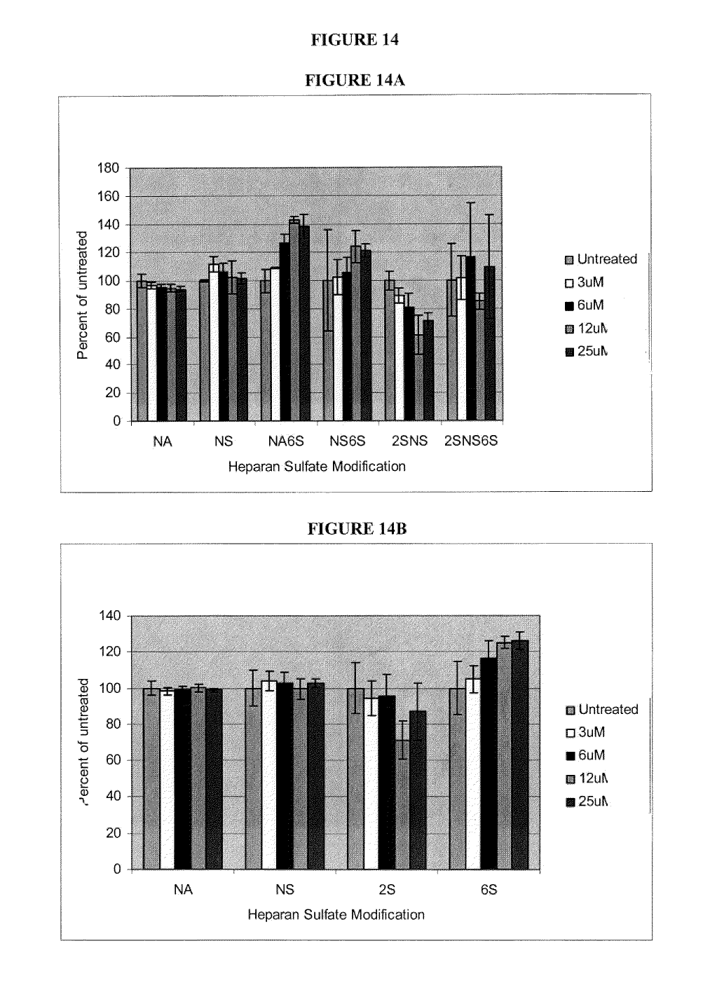

Substrate reduction therapy (SRT) is validated for MPS. The non-specific inhibitor of sulfation, sodium chlorate is used herein for such validation. 30-60 mM sodium chlorate inhibits the synthesis of PAPs, the sulfate donor used in all cellular sulfation reactions including heparan sulfate biosynthesis. Cells grown in the presence of sodium chlorate produce heparan sulfate with reduced sulfation. FIGS. 14A-C illustrates the reduction of GAG accumulation in in vitro models of MPS I, II, and IIIA. In certain instances, in vitro MPS models used herein are based on measuring the accumulation of GAG fragments in cultured primary human fibroblast from MPS patients. In some instances, the GAGs that accumulate in MPS patients are much smaller than normal tissue GAGs and they lack a core protein on their reducing termini. Based on these features the in vitro MPS model used in certain instances herein is based on a method of tagging reducing ends of the GAGs with a detectable label and analyzing (i.e., detecting and/or measuring) the detectable labels using a device suitable for detecting the label (e.g., an HPLC with a fluorimeter).

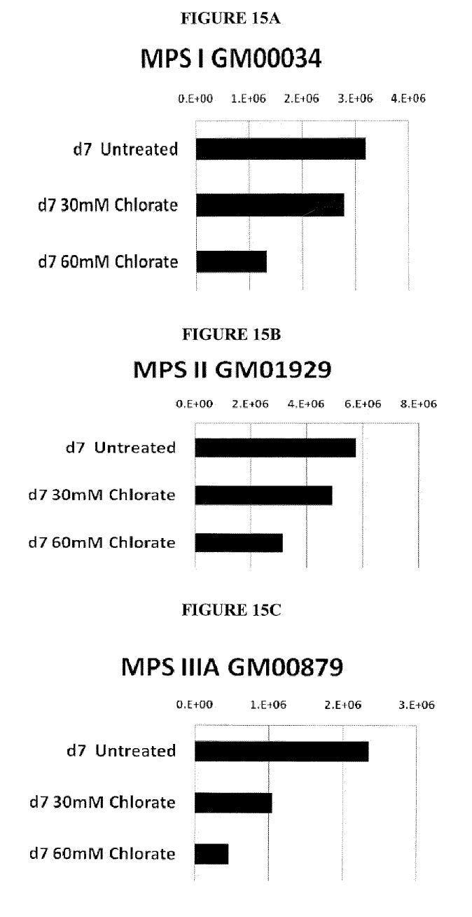

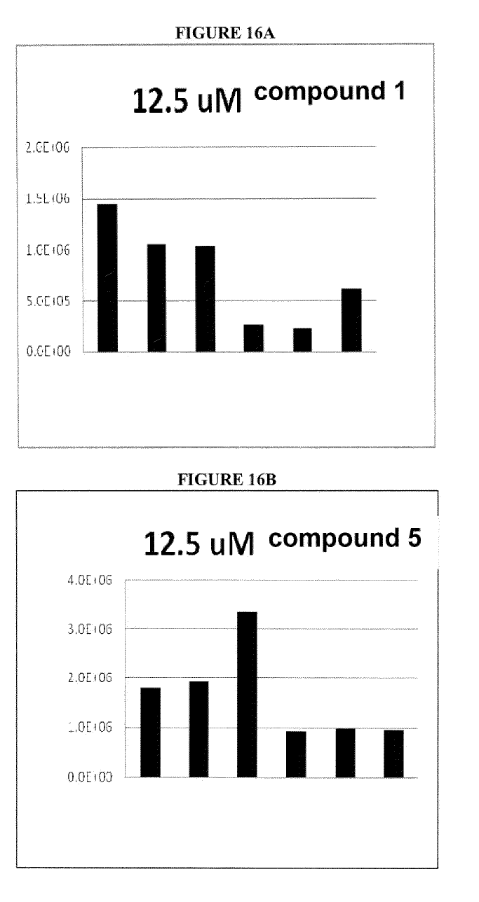

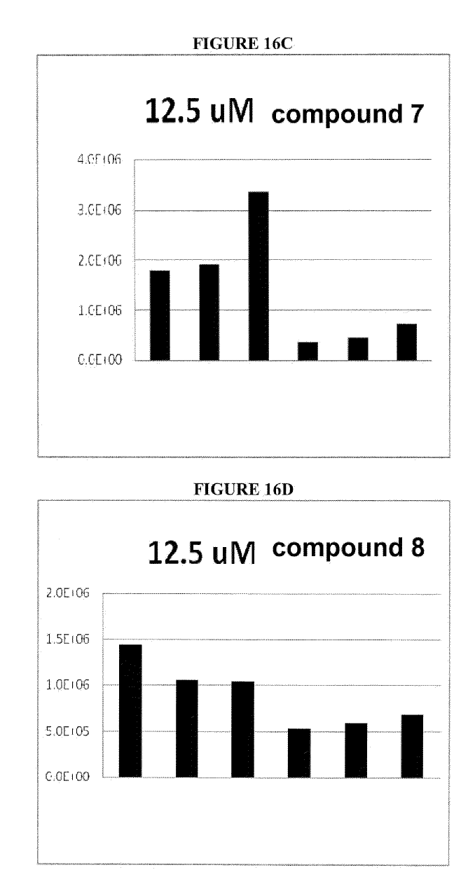

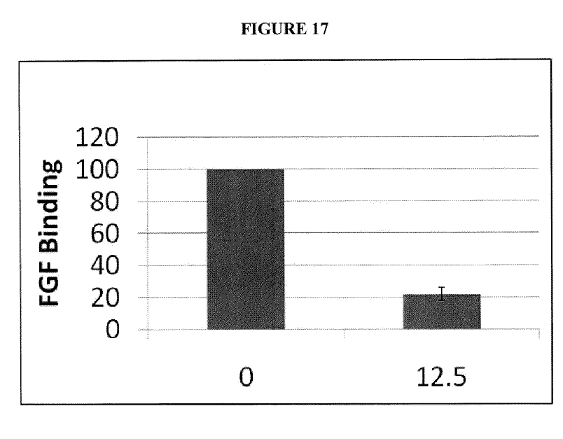

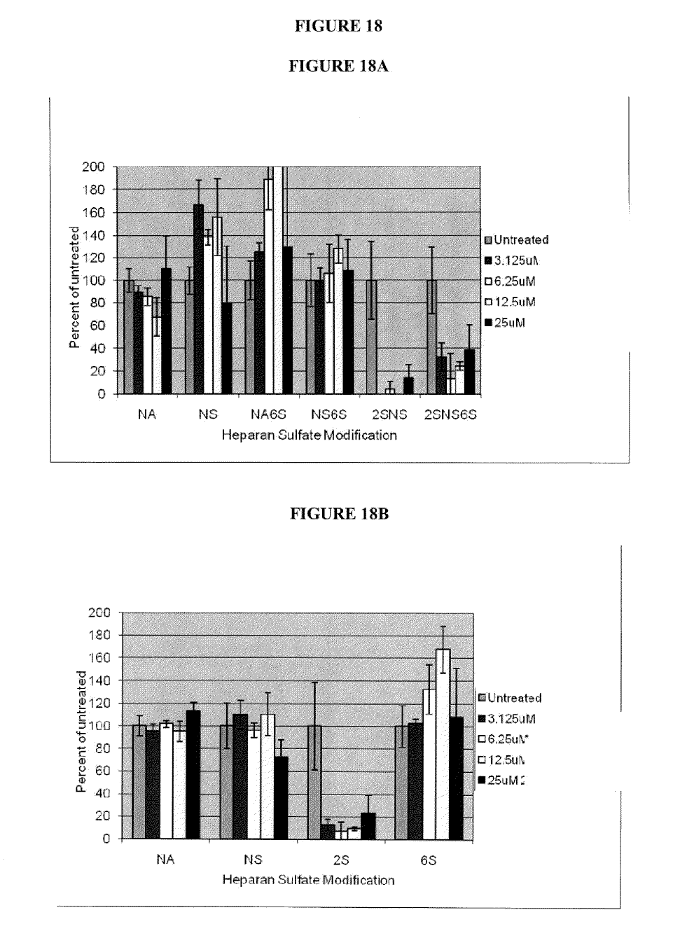

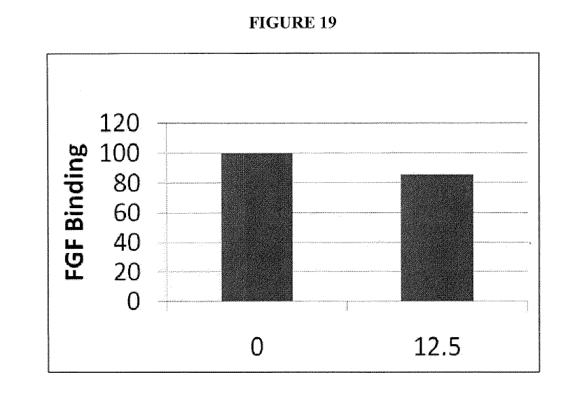

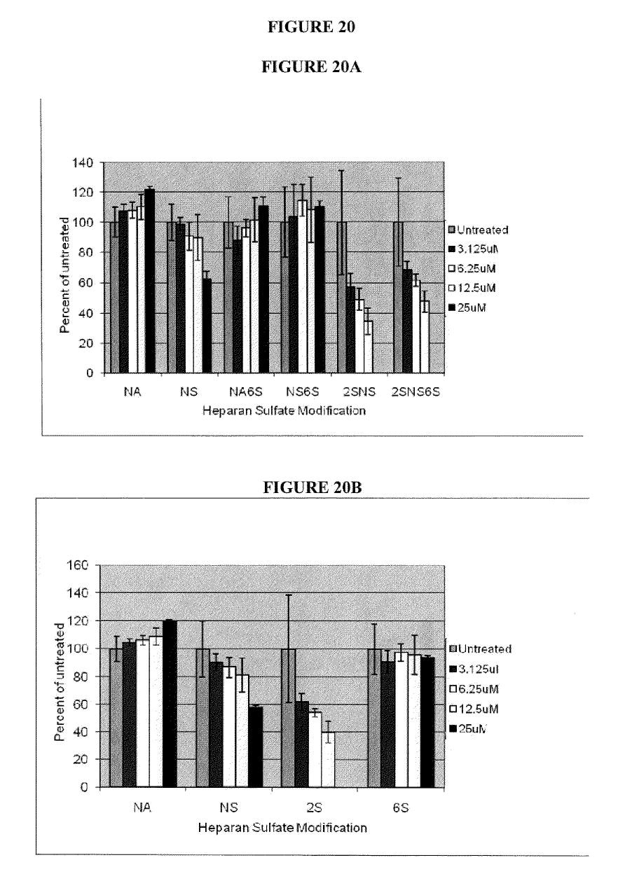

FIGS. 15A-D illustrate the inhibition of heparan sulfate biosynthesis with compounds 1, 5, 7, and 8, respectively. These compounds are illustrated to reduce GAG accumulation in human MPS IIIA fibroblasts.

### Example 12

### Method of Treatment

Human Clinical Trial of the Safety and/or Efficacy of selective heparan sulfate inhibitor (e.g., a compound of Formulas III-VII or FIG. 2, or a pharmaceutically acceptable salt thereof) therapy

Objective: To determine the safety and pharmacokinetics of administered selective heparan sulfate inhibitor (e.g., a compound of Formulas III-VII or FIG. 2, or a pharmaceutically acceptable salt thereof).

Study Design: This will be a Phase I, single-center, open-label, randomized dose escalation study followed by a Phase II study in lysosomal storage disease patients. Patients should not have had exposure to a selective heparan sulfate inhibitor prior to the study entry. Patients must not have received treatment for their lysosomal storage disease within 2 weeks of beginning the trial. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I: Patients receive (e.g., intravenous, oral, ip, or the like) selective heparan sulfate inhibitor (e.g., a compound of Formulas III-VII or FIG. 2, or a pharmaceutically acceptable salt thereof) q.i.d., t.i.d., b.i.d., or daily for 5 consecutive days or 7 days a week. Doses of selective heparan sulfate inhibitor (e.g., a compound of Formulas III-VII or FIG. 2, or a pharmaceutically acceptable salt thereof) may be held or modified for toxicity based on assessments as outlined below. Treatment repeats every 28 days in the absence of unacceptable toxicity. Cohorts of 3-6 patients receive escalating doses of selective heparan sulfate inhibitor (e.g., a compound of Formulas III-VII or FIG. 2, or a pharmaceutically acceptable salt thereof) until the maximum tolerated dose (MTD) for the selective heparan sulfate inhibitor (e.g., a compound of Formulas III-VII or FIG. 2, or a pharmaceutically acceptable salt thereof) is determined. The MTD is defined as the dose preceding that at which 2 of 3 or 2 of 6 patients experience dose-limiting toxicity. Dose limiting toxicities are determined in any suitable manner, e.g., according to the definitions and standards set by the National Cancer Institute (NCI) Common Terminology for Adverse Events (CTCAE) Version 3.0 (Aug. 9, 2006).

Phase II: Patients receive selective heparan sulfate inhibitor (e.g., a compound of Formulas III-VII or FIG. 2, or a pharmaceutically acceptable salt thereof) as in phase I at the MTD determined in phase I. Treatment repeats every 6 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they meet original eligibility criteria.

Blood Sampling: Serial blood is drawn by direct vein puncture before and after administration of selective heparan sulfate inhibitor (e.g., a compound of Formulas III-VII or FIG. 2, or a pharmaceutically acceptable salt thereof). Venous blood samples (5-10 mL) for determination of serum concentrations of selective heparan sulfate inhibitor are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics: Patients undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response/Urine Sampling: Urine is collected before and after administration of selective heparan sulfate inhibitor (e.g., a compound of Formulas III-VII or FIG. 2, or a pharmaceutically acceptable salt thereof) are obtained to determine GAG or heparan sulfate concentrations at about 10 minutes prior to dosing and at approximately the following times after dosing: days 1, 2, 3, 4, 5, 6, 7, 14, 28, 56, and 84. Additional testing may further be used to determine long term reduction, reduction in rate of increase, or maintenance of GAG or heparan sulfate levels. GAG or heparan sulfate concentrations can be determined in any suitable manner, e.g., based on a method of tagging reducing ends of the GAGs with a detectable label and analyzing (i.e., detecting and/or measuring) the detectable labels using a device suitable for detecting the label (e.g., an HPLC with a fluorimeter).

### Example 13

### Method of Treatment

Human Clinical Trial of the Safety and/or Efficacy of selective heparan sulfate inhibitor (e.g., a compound of Formulas III-VII or FIG. 2, or a pharmaceutically acceptable salt thereof) therapy

Objective: To determine the safety and pharmacokinetics of administered selective heparan sulfate inhibitor (e.g., a compound of Formulas III-VII or FIG. 2, or a pharmaceutically acceptable salt thereof).

Study Design: This will be a Phase I, single-center, open-label, randomized dose escalation study followed by a Phase II study in hyperheparansulfatemia patients. Patients should not have had exposure to a selective heparan sulfate inhibitor prior to the study entry. Patients must not have received treatment for their hyperheparansulfatemia within 2 weeks of beginning the trial. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I: Patients receive (e.g., intravenous, oral, ip, or the like) selective heparan sulfate inhibitor (e.g., a compound of Formulas III-VII or FIG. 2, or a pharmaceutically acceptable salt thereof) q.i.d., t.i.d., b.i.d., or daily for 5 consecutive days or 7 days a week. Doses of selective heparan sulfate inhibitor (e.g., a compound of Formulas III-VII or FIG. 2, or a pharmaceutically acceptable salt thereof) may be held or modified for toxicity based on assessments as outlined below. Treatment repeats every 28 days in the absence of unacceptable toxicity. Cohorts of 3-6 patients receive escalating doses of selective heparan sulfate inhibitor (e.g., a compound of Formulas III-VII or FIG. 2, or a pharmaceutically acceptable salt thereof) until the maximum tolerated dose (MTD) for the selective heparan sulfate inhibitor (e.g., a compound of Formulas III-VII or FIG. 2, or a pharmaceutically acceptable salt thereof) is determined. The MTD is defined as the dose preceding that at which 2 of 3 or 2 of 6 patients experience dose-limiting toxicity. Dose limiting toxicities are determined in any suitable manner, e.g., according to the definitions and standards set by the National Cancer Institute (NCI) Common Terminology for Adverse Events (CTCAE) Version 3.0 (Aug. 9, 2006).

Phase II: Patients receive selective heparan sulfate inhibitor (e.g., a compound of Formulas III-VII or FIG. 2, or a pharmaceutically acceptable salt thereof) as in phase I at the MTD determined in phase I. Treatment repeats every 6 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they meet original eligibility criteria.

Blood Sampling: Serial blood is drawn by direct vein puncture before and after administration of selective heparan sulfate inhibitor (e.g., a compound of Formulas III-VII or FIG. 2, or a pharmaceutically acceptable salt thereof). Venous blood samples (5-10 mL) for determination of serum concentrations of selective heparan sulfate inhibitor are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics: Patients undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response/Urine Sampling: Urine is collected before and after administration of selective heparan sulfate inhibitor (e.g., a compound of Formulas III-VII or FIG. 2, or a pharmaceutically acceptable salt thereof) are obtained to determine GAG or heparan sulfate concentrations at about 10 minutes prior to dosing and at approximately the following times after dosing: days 1, 2, 3, 4, 5, 6, 7, 14, 28, 56, and 84. Additional testing may further be used to determine long term reduction, reduction in rate of increase, or maintenance of GAG or heparan sulfate levels. GAG or heparan sulfate concentrations can be determined in any suitable manner, e.g., based on a method of tagging reducing ends of the GAGs with a detectable label and analyzing (i.e., detecting and/or measuring) the detectable labels using a device suitable for detecting the label (e.g., an HPLC with a fluorimeter).

### Example 14

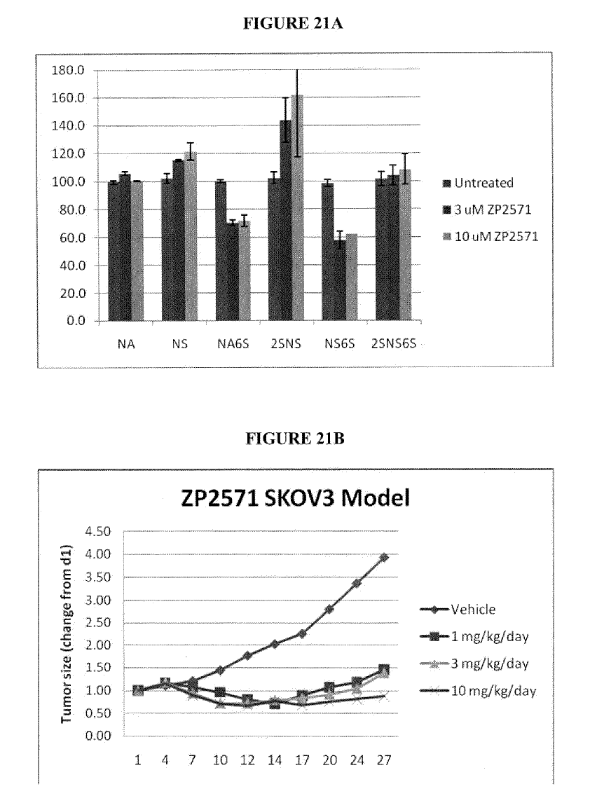

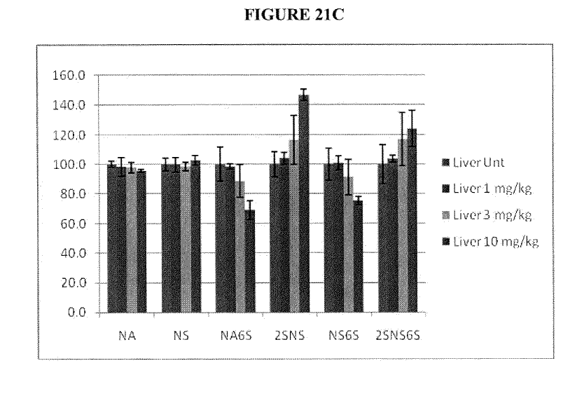

The change in the structure of heparan sulfate induced by compound 9 suggests that it inhibits the 6-O sulfation of heparan sulfate (See FIG. 21A):

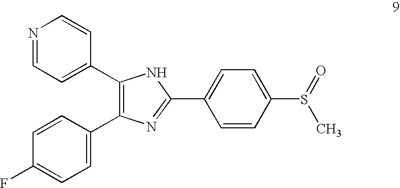

Compound 9 was tested for anti-cancer activity in a Human ovarian cancer model. Briefly, 5 million SKOV3 cells were injected in 100 ul mixed with matrigel subcutaneously into female nude mice. The tumors were allowed to grow to 0.1 cm³ then the mice were randomized into four groups (Vehicle, 1, 3, and 10 mg/kg/day). Each group received the indicated daily dose divided into two IP injections. Vehicle alone received the formulation only. As shown in FIG. 21B, the compound blocked tumor growth at all doses tested.

After sacrifice, the liver and tumor were analyzed for changes in the structure of heparan sulfate. No changes were detected in the small tumor that remained in the treated animals; however, heparan sulfate biosynthesis was altered in the liver of treated animals in a dose dependent manner. As seen in FIG. 21C (comparing to FIG. 21A), these changes are identical to those observed in cultured human cells treated with compound 9.

### Example 15

### Ovarian Cancer Model

Selective heparan sulfate inhibitor is tested for anti-cancer activity in a Human ovarian cancer model. Briefly, 5 million SKOV3 cells are injected in 100 ul mixed with matrigel subcutaneously into female nude mice. The tumors are allowed to grow to 0.1 cm³ then the mice are randomized into four groups (Vehicle, 1, 3, and 10 mg/kg/day). Each group receives the indicated daily dose divided into two IP injections. Vehicle alone receive the formulation only. Tumor growth is measured at days 1, 4, 7, 10, 12, 14, 17, 20, 24, and 27. After sacrifice, the liver and tumor are analyzed for changes in the structure of heparan sulfate.

### Example 16

### Pancreatic Cancer Model

Selective heparan sulfate inhibitor is tested for anti-cancer activity in a Human pancreatic cancer model. Briefly, 5 million PANC-1 or COLO-357 cells are injected in 100 ul mixed with matrigel subcutaneously into female nude mice. The tumors are allowed to grow to 0.1 cm³ then the mice are randomized into four groups (Vehicle, 1, 3, and 10 mg/kg/day). Each group receives the indicated daily dose divided into two IP injections. Vehicle alone receive the formulation only. Tumor growth is measured at days 1, 4, 7, 10, 12, 14, 17, 20, 24, and 27. After sacrifice, the liver and tumor are analyzed for changes in the structure of heparan sulfate.

### Example 17

### Breast Cancer Model

Selective heparan sulfate inhibitor is tested for anti-cancer activity in a Human breast cancer model. Briefly, 5 million MCF-7 cells are injected in 100 ul mixed with matrigel subcutaneously into female nude mice. The tumors are allowed to grow to 0.1 cm³ then the mice are randomized into four groups (Vehicle, 1, 3, and 10 mg/kg/day). Each group receives the indicated daily dose divided into two IP injections. Vehicle alone receive the formulation only. Tumor growth is measured at days 1, 4, 7, 10, 12, 14, 17, 20, 24, and 27. After sacrifice, the liver and tumor are analyzed for changes in the structure of heparan sulfate.

### Example 18

### Lung Cancer Model

Selective heparan sulfate inhibitor is tested for anti-cancer activity in a Human lung cancer model. Briefly, 5 million cells of a lung cancer cell line (e.g., H460, H23, HTB-58, A549, H441, or H2170) are injected in 100 ul mixed with matrigel subcutaneously into female nude mice. The tumors are allowed to grow to 0.1 cm³ then the mice are randomized into four groups (Vehicle, 1, 3, and 10 mg/kg/day). Each group receives the indicated daily dose divided into two IP injections. Vehicle alone receive the formulation only. Tumor growth is measured at days 1, 4, 7, 10, 12, 14, 17, 20, 24, and 27. After sacrifice, the liver and tumor are analyzed for changes in the structure of heparan sulfate.

### Example 19

### Colon Cancer Model

Selective heparan sulfate inhibitor is tested for anti-cancer activity in a Human colon cancer model. Briefly, 5 million HT-29 cells are injected in 100 ul mixed with matrigel subcutaneously into female nude mice. The tumors are allowed to grow to 0.1 cm³ then the mice are randomized into four groups (Vehicle, 1, 3, and 10 mg/kg/day). Each group receives the indicated daily dose divided into two IP injections. Vehicle alone receive the formulation only. Tumor growth is measured at days 1, 4, 7, 10, 12, 14, 17, 20, 24, and 27. After sacrifice, the liver and tumor are analyzed for changes in the structure of heparan sulfate.

### Example 20

### Prostate Cancer Model

Selective heparan sulfate inhibitor is tested for anti-cancer activity in a Human prostate cancer model. Briefly, 5 million cells from a prostate cancer cell line (e.g., PC3 or LNCaP) are injected in 100 ul mixed with matrigel subcutaneously into female nude mice. The tumors are allowed to grow to 0.1 cm³ then the mice are randomized into four groups (Vehicle, 1, 3, and 10 mg/kg/day). Each group receives the indicated daily dose divided into two IP injections. Vehicle alone receive the formulation only. Tumor growth is measured at days 1, 4, 7, 10, 12, 14, 17, 20, 24, and 27. After sacrifice, the liver and tumor are analyzed for changes in the structure of heparan sulfate.

### Example 21

### Method of Treating Ovarian Cancer

Human Clinical Trial of the Safety and/or Efficacy of selective heparan sulfate inhibitor therapy (e.g., with a compound of any of Formulas III-VII or FIG. 2)

Objective: To determine the safety and pharmacokinetics of administered selective heparan sulfate inhibitor therapy (e.g., with a compound of any of Formulas III-VII or FIG. 2).

Study Design: This will be a Phase I, single-center, open-label, randomized dose escalation study followed by a Phase II study in ovarian cancer patients. Patients should not have had exposure to selective heparan sulfate inhibitor (e.g., with a compound of any of Formulas III-VII or FIG. 2) prior to the study entry. Patients must not have received treatment for their cancer within 2 weeks of beginning the trial. Treatments include the use of chemotherapy, hematopoietic growth factors, and biologic therapy such as monoclonal antibodies. The exception is the use of hydroxyurea for patients with WBC >30×103/μL. This duration of time appears adequate for wash out due to the relatively short-acting nature of most anti-leukemia agents. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I: Patients receive selective heparan sulfate inhibitor (e.g., with a compound of any of Formulas III-VII or FIG. 2) in a suitable manner (e.g., IP, intravenously, orally, rectally, or the like) daily for 5 consecutive days or 7 days a week. Doses of selective heparan sulfate inhibitor (e.g., with a compound of any of Formulas III-VII or FIG. 2) may be held or modified for toxicity based on assessments as outlined below. Treatment repeats every 28 days in the absence of unacceptable toxicity. Cohorts of 3-6 patients receive escalating doses of selective heparan sulfate inhibitor (e.g., with a compound of any of Formulas III-VII or FIG. 2) until the maximum tolerated dose (MTD) for the selective heparan sulfate inhibitor (e.g., with a compound of any of Formulas III-VII or FIG. 2) is determined. The MTD is defined as the dose preceding that at which 2 of 3 or 2 of 6 patients experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II: Patients receive selective heparan sulfate inhibitor (e.g., with a compound of any of Formulas III-VII or FIG. 2) as in phase I at the MTD determined in phase I. Treatment repeats every 6 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they meet original eligibility criteria.

Blood Sampling Serial blood is drawn by direct vein puncture before and after administration of selective heparan sulfate inhibitor (e.g., with a compound of any of Formulas III-VII or FIG. 2). Venous blood samples (5 mL) for determination of serum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics: Patients undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response: Patient response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon the cancer type and feasibility/availability, and the same imaging modality is utilized for similar cancer types as well as throughout each patient's study course. Response rates are determined using the RECIST criteria. (Therasse et al, J. Natl. Cancer Inst. 2000 Feb. 2; 92(3):205-16; http://ctep.cancer.gov/forms/TherasseRECISTJNCI.pdf). Patients also undergo cancer/tumor biopsy to assess changes in progenitor cancer cell phenotype and clonogenic growth by flow cytometry, Western blotting, and IHC, and for changes in cytogenetics by FISH or TaqMan PCR for specific chromosomal translocations. After completion of study treatment, patients are followed periodically for 4 weeks.

### Example 22

### Method of Treating Pancreatic Cancer

Human Clinical Trial of the Safety and/or Efficacy of selective heparan sulfate inhibitor therapy (e.g., with a compound of any of Formulas III-VII or FIG. 2)

Objective: To determine the safety and pharmacokinetics of administered selective heparan sulfate inhibitor therapy (e.g., with a compound of any of Formulas III-VII or FIG. 2).

Study Design: This will be a Phase I, single-center, open-label, randomized dose escalation study followed by a Phase II study in pancreatic cancer patients. Patients should not have had exposure to selective heparan sulfate inhibitor (e.g., with a compound of any of Formulas III-VII or FIG. 2) prior to the study entry. Patients must not have received treatment for their cancer within 2 weeks of beginning the trial. Treatments include the use of chemotherapy, hematopoietic growth factors, and biologic therapy such as monoclonal antibodies. The exception is the use of hydroxyurea for patients with WBC >30×103/μL. This duration of time appears adequate for wash out due to the relatively short-acting nature of most anti-leukemia agents. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I: Patients receive selective heparan sulfate inhibitor (e.g., with a compound of any of Formulas III-VII or FIG. 2) in a suitable manner (e.g., IP, intravenously, orally, rectally, or the like) daily for 5 consecutive days or 7 days a week. Doses of selective heparan sulfate inhibitor (e.g., with a compound of any of Formulas III-VII or FIG. 2) may be held or modified for toxicity based on assessments as outlined below. Treatment repeats every 28 days in the absence of unacceptable toxicity. Cohorts of 3-6 patients receive escalating doses of selective heparan sulfate inhibitor (e.g., with a compound of any of Formulas III-VII or FIG. 2) until the maximum tolerated dose (MTD) for the selective heparan sulfate inhibitor (e.g., with a compound of any of Formulas III-VII or FIG. 2) is determined. The MTD is defined as the dose preceding that at which 2 of 3 or 2 of 6 patients experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II: Patients receive selective heparan sulfate inhibitor (e.g., with a compound of any of Formulas III-VII or FIG. 2) as in phase I at the MTD determined in phase I. Treatment repeats every 6 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they meet original eligibility criteria.

Blood Sampling Serial blood is drawn by direct vein puncture before and after administration of selective heparan sulfate inhibitor (e.g., with a compound of any of Formulas III-VII or FIG. 2). Venous blood samples (5 mL) for determination of serum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics: Patients undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response: Patient response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon the cancer type and feasibility/availability, and the same imaging modality is utilized for similar cancer types as well as throughout each patient's study course. Response rates are determined using the RECIST criteria. (Therasse et al, J. Natl. Cancer Inst. 2000 Feb. 2; 92(3):205-16; http://ctep.cancer.gov/forms/TherasseRECISTJNCI.pdf). Patients also undergo cancer/tumor biopsy to assess changes in progenitor cancer cell phenotype and clonogenic growth by flow cytometry, Western blotting, and IHC, and for changes in cytogenetics by FISH or TaqMan PCR for specific chromosomal translocations. After completion of study treatment, patients are followed periodically for 4 weeks.

### Example 23

### Method of Treating Breast Cancer

Human Clinical Trial of the Safety and/or Efficacy of selective heparan sulfate inhibitor therapy (e.g., with a compound of any of Formulas III-VII or FIG. 2)

Objective: To determine the safety and pharmacokinetics of administered selective heparan sulfate inhibitor therapy (e.g., with a compound of any of Formulas III-VII or FIG. 2).

Study Design: This will be a Phase I, single-center, open-label, randomized dose escalation study followed by a Phase II study in breast cancer patients. Patients should not have had exposure to selective heparan sulfate inhibitor (e.g., with a compound of any of Formulas III-VII or FIG. 2) prior to the study entry. Patients must not have received treatment for their cancer within 2 weeks of beginning the trial. Treatments include the use of chemotherapy, hematopoietic growth factors, and biologic therapy such as monoclonal antibodies. The exception is the use of hydroxyurea for patients with WBC >30×103/μL. This duration of time appears adequate for wash out due to the relatively short-acting nature of most anti-leukemia agents. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I: Patients receive selective heparan sulfate inhibitor (e.g., with a compound of any of Formulas III-VII or FIG. 2) in a suitable manner (e.g., IP, intravenously, orally, rectally, or the like) daily for 5 consecutive days or 7 days a week. Doses of selective heparan sulfate inhibitor (e.g., with a compound of any of Formulas III-VII or FIG. 2) may be held or modified for toxicity based on assessments as outlined below. Treatment repeats every 28 days in the absence of unacceptable toxicity. Cohorts of 3-6 patients receive escalating doses of selective heparan sulfate inhibitor (e.g., with a compound of any of Formulas III-VII or FIG. 2) until the maximum tolerated dose (MTD) for the selective heparan sulfate inhibitor (e.g., with a compound of any of Formulas III-VII or FIG. 2) is determined. The MTD is defined as the dose preceding that at which 2 of 3 or 2 of 6 patients experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II: Patients receive selective heparan sulfate inhibitor (e.g., with a compound of any of Formulas III-VII or FIG. 2) as in phase I at the MTD determined in phase I. Treatment repeats every 6 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they meet original eligibility criteria.

Blood Sampling Serial blood is drawn by direct vein puncture before and after administration of selective heparan sulfate inhibitor (e.g., with a compound of any of Formulas III-VII or FIG. 2). Venous blood samples (5 mL) for determination of serum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics: Patients undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response: Patient response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon the cancer type and feasibility/availability, and the same imaging modality is utilized for similar cancer types as well as throughout each patient's study course. Response rates are determined using the RECIST criteria. (Therasse et al, J. Natl. Cancer Inst. 2000 Feb. 2; 92(3):205-16; http://ctep.cancer.gov/forms/TherasseRECISTJNCI.pdf). Patients also undergo cancer/tumor biopsy to assess changes in progenitor cancer cell phenotype and clonogenic growth by flow cytometry, Western blotting, and IHC, and for changes in cytogenetics by FISH or TaqMan PCR for specific chromosomal translocations. After completion of study treatment, patients are followed periodically for 4 weeks.

### Example 24

### Method of Treating Lung Cancer

Human Clinical Trial of the Safety and/or Efficacy of selective heparan sulfate inhibitor therapy (e.g., with a compound of any of Formulas III-VII or FIG. 2)

Objective: To determine the safety and pharmacokinetics of administered selective heparan sulfate inhibitor therapy (e.g., with a compound of any of Formulas III-VII or FIG. 2).

Study Design: This will be a Phase I, single-center, open-label, randomized dose escalation study followed by a Phase II study in lung cancer patients. Patients should not have had exposure to selective heparan sulfate inhibitor (e.g., with a compound of any of Formulas III-VII or FIG. 2) prior to the study entry. Patients must not have received treatment for their cancer within 2 weeks of beginning the trial. Treatments include the use of chemotherapy, hematopoietic growth factors, and biologic therapy such as monoclonal antibodies. The exception is the use of hydroxyurea for patients with WBC >30×103/μL. This duration of time appears adequate for wash out due to the relatively short-acting nature of most anti-leukemia agents. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I: Patients receive selective heparan sulfate inhibitor (e.g., with a compound of any of Formulas III-VII or FIG. 2) in a suitable manner (e.g., IP, intravenously, orally, rectally, or the like) daily for 5 consecutive days or 7 days a week. Doses of selective heparan sulfate inhibitor (e.g., with a compound of any of Formulas III-VII or FIG. 2) may be held or modified for toxicity based on assessments as outlined below. Treatment repeats every 28 days in the absence of unacceptable toxicity. Cohorts of 3-6 patients receive escalating doses of selective heparan sulfate inhibitor (e.g., with a compound of any of Formulas III-VII or FIG. 2) until the maximum tolerated dose (MTD) for the selective heparan sulfate inhibitor (e.g., with a compound of any of Formulas III-VII or FIG. 2) is determined. The MTD is defined as the dose preceding that at which 2 of 3 or 2 of 6 patients experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II: Patients receive selective heparan sulfate inhibitor (e.g., with a compound of any of Formulas III-VII or FIG. 2) as in phase I at the MTD determined in phase I. Treatment repeats every 6 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they meet original eligibility criteria.

Blood Sampling Serial blood is drawn by direct vein puncture before and after administration of selective heparan sulfate inhibitor (e.g., with a compound of any of Formulas III-VII or FIG. 2). Venous blood samples (5 mL) for determination of serum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics: Patients undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response: Patient response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon the cancer type and feasibility/availability, and the same imaging modality is utilized for similar cancer types as well as throughout each patient's study course. Response rates are determined using the RECIST criteria. (Therasse et al, J. Natl. Cancer Inst. 2000 Feb. 2; 92(3):205-16; http://ctep.cancer.gov/forms/TherasseRECISTJNCI.pdf). Patients also undergo cancer/tumor biopsy to assess changes in progenitor cancer cell phenotype and clonogenic growth by flow cytometry, Western blotting, and IHC, and for changes in cytogenetics by FISH or TaqMan PCR for specific chromosomal translocations. After completion of study treatment, patients are followed periodically for 4 weeks.

### Example 25

### Method of Treating Colon Cancer

Human Clinical Trial of the Safety and/or Efficacy of selective heparan sulfate inhibitor therapy (e.g., with a compound of any of Formulas III-VII or FIG. 2)

Objective: To determine the safety and pharmacokinetics of administered selective heparan sulfate inhibitor therapy (e.g., with a compound of any of Formulas III-VII or FIG. 2).

Study Design: This will be a Phase I, single-center, open-label, randomized dose escalation study followed by a Phase II study in colon cancer patients. Patients should not have had exposure to selective heparan sulfate inhibitor (e.g., with a compound of any of Formulas III-VII or FIG. 2) prior to the study entry. Patients must not have received treatment for their cancer within 2 weeks of beginning the trial. Treatments include the use of chemotherapy, hematopoietic growth factors, and biologic therapy such as monoclonal antibodies. The exception is the use of hydroxyurea for patients with WBC >30×103/μL. This duration of time appears adequate for wash out due to the relatively short-acting nature of most anti-leukemia agents. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I: Patients receive selective heparan sulfate inhibitor (e.g., with a compound of any of Formulas III-VII or FIG. 2) in a suitable manner (e.g., IP, intravenously, orally, rectally, or the like) daily for 5 consecutive days or 7 days a week. Doses of selective heparan sulfate inhibitor (e.g., with a compound of any of Formulas III-VII or FIG. 2) may be held or modified for toxicity based on assessments as outlined below. Treatment repeats every 28 days in the absence of unacceptable toxicity. Cohorts of 3-6 patients receive escalating doses of selective heparan sulfate inhibitor (e.g., with a compound of any of Formulas III-VII or FIG. 2) until the maximum tolerated dose (MTD) for the selective heparan sulfate inhibitor (e.g., with a compound of any of Formulas III-VII or FIG. 2) is determined. The MTD is defined as the dose preceding that at which 2 of 3 or 2 of 6 patients experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II: Patients receive selective heparan sulfate inhibitor (e.g., with a compound of any of Formulas III-VII or FIG. 2) as in phase I at the MTD determined in phase I. Treatment repeats every 6 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they meet original eligibility criteria.

Blood Sampling Serial blood is drawn by direct vein puncture before and after administration of selective heparan sulfate inhibitor (e.g., with a compound of any of Formulas III-VII or FIG. 2). Venous blood samples (5 mL) for determination of serum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics: Patients undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response: Patient response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon the cancer type and feasibility/availability, and the same imaging modality is utilized for similar cancer types as well as throughout each patient's study course. Response rates are determined using the RECIST criteria. (Therasse et al, J. Natl. Cancer Inst. 2000 Feb. 2; 92(3):205-16; http://ctep.cancer.gov/forms/TherasseRECISTJNCI.pdf). Patients also undergo cancer/tumor biopsy to assess changes in progenitor cancer cell phenotype and clonogenic growth by flow cytometry, Western blotting, and IHC, and for changes in cytogenetics by FISH or TaqMan PCR for specific chromosomal translocations. After completion of study treatment, patients are followed periodically for 4 weeks.

### Example 26

### Method of Treating Prostate Cancer

Human Clinical Trial of the Safety and/or Efficacy of selective heparan sulfate inhibitor therapy (e.g., with a compound of any of Formulas III-VII or FIG. 2)

Objective: To determine the safety and pharmacokinetics of administered selective heparan sulfate inhibitor therapy (e.g., with a compound of any of Formulas III-VII or FIG. 2).

Study Design: This will be a Phase I, single-center, open-label, randomized dose escalation study followed by a Phase II study in prostate cancer patients. Patients should not have had exposure to selective heparan sulfate inhibitor (e.g., with a compound of any of Formulas III-VII or FIG. 2) prior to the study entry. Patients must not have received treatment for their cancer within 2 weeks of beginning the trial. Treatments include the use of chemotherapy, hematopoietic growth factors, and biologic therapy such as monoclonal antibodies. The exception is the use of hydroxyurea for patients with WBC >30×103/μL. This duration of time appears adequate for wash out due to the relatively short-acting nature of most anti-leukemia agents. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I: Patients receive selective heparan sulfate inhibitor (e.g., with a compound of any of Formulas III-VII or FIG. 2) in a suitable manner (e.g., IP, intravenously, orally, rectally, or the like) daily for 5 consecutive days or 7 days a week. Doses of selective heparan sulfate inhibitor (e.g., with a compound of any of Formulas III-VII or FIG. 2) may be held or modified for toxicity based on assessments as outlined below. Treatment repeats every 28 days in the absence of unacceptable toxicity. Cohorts of 3-6 patients receive escalating doses of selective heparan sulfate inhibitor (e.g., with a compound of any of Formulas III-VII or FIG. 2) until the maximum tolerated dose (MTD) for the selective heparan sulfate inhibitor (e.g., with a compound of any of Formulas III-VII or FIG. 2) is determined. The MTD is defined as the dose preceding that at which 2 of 3 or 2 of 6 patients experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II: Patients receive selective heparan sulfate inhibitor (e.g., with a compound of any of Formulas III-VII or FIG. 2) as in phase I at the MTD determined in phase I. Treatment repeats every 6 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they meet original eligibility criteria.

Blood Sampling Serial blood is drawn by direct vein puncture before and after administration of selective heparan sulfate inhibitor (e.g., with a compound of any of Formulas III-VII or FIG. 2). Venous blood samples (5 mL) for determination of serum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics: Patients undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response: Patient response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon the cancer type and feasibility/availability, and the same imaging modality is utilized for similar cancer types as well as throughout each patient's study course. Response rates are determined using the RECIST criteria. (Therasse et al, J. Natl. Cancer Inst. 2000 Feb. 2; 92(3):205-16; http://ctep.cancer.gov/forms/TherasseRECISTJNCI.pdf). Patients also undergo cancer/tumor biopsy to assess changes in progenitor cancer cell phenotype and clonogenic growth by flow cytometry, Western blotting, and IHC, and for changes in cytogenetics by FISH or TaqMan PCR for specific chromosomal translocations. After completion of study treatment, patients are followed periodically for 4 weeks.

What is claimed is:

1. A method of treating cancer comprising administering a therapeutically effective amount of a selective modulator of heparan sulfate glycosylation, a modulator of heparan sulfate sulfation, or a selective modulator of heparan sulfate epimerization.

2. The process of claim 1, wherein the selective modulator of heparan sulfate biosynthesis inhibits heparan glycosylation.

3. The process of claim 1, wherein the selective modulator of heparan sulfate biosynthesis inhibits sulfation of heparan.

4. The process of claim 1, wherein the selective modulator of heparan sulfate biosynthesis promotes sulfation of heparan.

5. The process of claim 1, wherein the selective modulator of heparan sulfate biosynthesis inhibits epimerization of heparan.

6. The process of claim 1, wherein the selective modulator of heparan sulfate biosynthesis promotes epimerization of heparan.

7. The process of claim 1, wherein the selective modulator of heparan sulfate is an inhibitor of 6-O sulfation.

8. The process of any of claim 1, wherein the selective modulator of heparan sulfate biosynthesis is a non-carbohydrate having a molecular weight of less than 1,000 g/mol.

9. A method of treating a lysosomal storage disease comprising administering a therapeutically effective amount of a selective modulator of heparan sulfate glycosylation, a modulator of heparan sulfate sulfation, or a selective modulator of heparan sulfate epimerization.

10. The method of claim 9, wherein the lysosomal storage disease is selected from mucopolysaccharidosis.

11. The method of claim 9, wherein the selective modulator of heparan sulfate glycosylation is an inhibitor of heparan sulfate glycosylation.

12. The method of claim 9, wherein the selective modulator of heparan sulfate sulfation is an inhibitor of heparan sulfate sulfation.

13. The method of claim 9, wherein the selective modulator of heparan sulfate epimerization is an inhibitor of heparan sulfate epimerization.

14. The process of claim 9, wherein the selective modulator of heparan sulfate glycosylation, a modulator of heparan sulfate sulfation, or a selective modulator of heparan sulfate epimerization is an inhibitor of 6-O sulfation.

15. The process of claim 9, wherein the selective modulator of heparan sulfate glycosylation, a modulator of heparan sulfate sulfation, or a selective modulator of heparan sulfate epimerization is a non-carbohydrate having a molecular weight of less than 1,000 g/mol.

16. A compound having the structure: 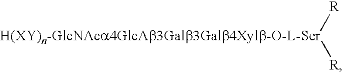
wherein:
each R is independently H or at least one amino acid,
n is 1-300;
each X is: 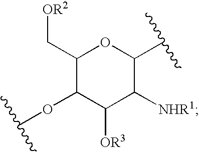
R¹ is H, COCH₃, or SO₃R⁵;
R² is H, or SO₃R⁵;
R³ is H, or SO₃R⁵;
each Y is: 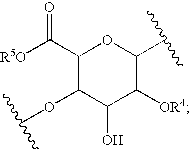
R⁴ is H, or SO₃R⁵;
each R⁵ is independently selected from H and a negative charge;
or physiologically acceptable salts thereof,
and wherein the substituents have one or more of the following ratios:
R¹═SO₃R⁵ to R¹═COCH₃ is about 0:1 to about 0.2:1;
R²═SO₃R⁵ to R²═H is about 0:1 to about 0.1:1;
R⁴═SO₃R⁵ to R²═SO₃R is about 0:1 to about 0.7:1,
R⁴═SO₃R⁵ to R²═SO₃R⁵ is >1.05; or
R⁴═SO₃R⁵ to R⁴═H is about 0:1 to about 0.1:1.

17. The compound of claim 16, wherein the ratio of R⁴═SO₃R⁵ to R²═SO₃R⁵ is about 0:1 to about 0.7:1.

18. The compound of claim 16, wherein the ratio of R⁴═SO₃R⁵ to R²═SO₃R⁵ is about 0:1 to about 0.5:1.

19. The compound of claim 16, wherein the ratio of R⁴═SO₃R⁵ to R²═SO₃R⁵ is about 0:1 to about 0.4:1.

20. The compound of claim 16, wherein the ratio of R⁴═SO₃R⁵ to R²═SO₃R⁵ is >1.05.

21. The compound of claim 16, wherein the ratio of R⁴═SO₃R⁵ to R²═SO₃R⁵ is >1.2.

22. The compound of claim 16, wherein less than 10% of R⁴═SO₃R⁵.

23. The compound of claim 16, wherein each R is at least one amino acid.
